(19) United States
(12) Patent Application Publication
(10) Pub. No.: US 2026/0284219 A1
(43) Pub. Date: Sep. 24, 2026

(54) ANTIBODY CONJUGATE, AND USES THEREOF

(71) Applicant: Acepodia Biotechnologies, Ltd., Taipei City (TW)

(72) Inventors: Shih-Chia HSIAO, Taipei City (TW); Hao-Kang LI, Taipei City (TW); Cheng-Wei LAI, Taipei City (TW)

(73) Assignee: Acepodia Biotechnologies, Ltd., Taipei City (TW)

(21) Appl. No.: 19/677,612

(22) Filed: May 14, 2026

Related U.S. Application Data
(63) Continuation-in-part of application No. PCT/US2025/031110, filed on May 28, 2025.
(60) Provisional application No. 63/653,254, filed on May 30, 2024.

(51) Int. Cl. A61K 47/68; A61P 35/00

(52) U.S. Cl. CPC A61K 47/6851; A61K 47/68035; A61K 47/68037; A61K 47/6811; A61K 47/6889; A61P 35/00

(57) ABSTRACT

An antibody conjugate, pharmaceutical composition, and treating and manufacturing method is related to a conjugate including an antibody or antigen-binding fragment thereof, a first functional unit, and a second functional unit. The antibody or antigen-binding fragment thereof includes a light chain having first and second amino acid residues of different types, and a heavy chain having at least one third amino acid residue of a same type as the first amino acid residue. The first functional unit has a formula -L1-D1, and the second functional unit has a formula -L2-D2. D1 and D2 are the functional moieties, respectively. L1 is a first linker residue connecting D1 to the first amino acid residue, or to both the first and the at least one third amino acid residues. L2 is a second linker residue connecting D2 to the second amino acid residue.

### CROSS-REFERENCE TO RELATED APPLICATION

The present application is a continuation-in-part of international application No. PCT/US2025/031110, filed on May 28, 2025, which claims the priority of U.S. provisional application No. 63/653,254, filed on May 30, 2024, the entirety of which are incorporated herein by reference.

### BACKGROUND

### Technical Field

The present invention relates to an antibody or antigen-binding fragment thereof covalently linked to at least two different functional units, and uses thereof; more particularly relates to an antibody conjugate including an antibody or antigen-binding fragment thereof, wherein a light chain of the antibody or antigen-binding fragment thereof is covalently linked to at least two different functional units, and uses thereof.

### Related Art

Antibody conjugates, such as antibody drug conjugate (ADC), are designed as targeted agents. Antibody drug conjugate (ADC) typically includes an antibody, a payload (drug), and a chemical linker, wherein the drug is covalently linked to the antibody via the chemical linker.

Drug antibody ratio (DAR value) is defined as the number of drug molecules conjugated to an antibody. ADC with low DAR means fewer drug molecules are attached to an antibody; the low-DAR ADCs exhibit limited overall cytotoxic effect on target cells, especially problematic in tumors with high heterogeneity or lower antigen expression.

Single-payload ADCs deliver only one cytotoxic drug to target cells. However, single-payload ADCs have limitations. Take tumor heterogeneity as an example, tumors often consist of a mix of cells with different sensitivities to drugs. A single-payload ADC having only one specific payload mechanism may not effectively attack diverse abnormal cell subpopulations within a tumor with varied mechanisms. Therefore, there's a greater chance that residual tumor cells could survive and lead to disease recurrence. Moreover, if cancer cells develop resistance to that specific payload mechanism, the ADC may become less effective or even ineffective.

Accordingly, there is an unmet need for high-DAR ADCs and dual-payload ADCs with improved therapeutic potential. The present disclosures address this need and provide related advantages.

### SUMMARY

In some embodiments, a conjugate includes:
an antibody or an antigen-binding fragment thereof, comprising a light chain having a first amino acid residue and a second amino acid residue, and a heavy chain having at least one third amino acid residue;
a first functional unit having a formula -L1-D1 conjugated to the antibody or antigen-binding fragment thereof via the first amino acid residue, or via both the first and the at least one third amino acid residues; and
a second functional unit having a formula -L2-D2 conjugated to the antibody or antigen-binding fragment thereof via the second amino acid residue,
wherein
D1 and D2 are each independently a functional moiety;
L1 is a first linker residue connecting D1 to the first amino acid residue, or to both the first and the at least one third amino acid residues;
L2 is a second linker residue connecting D2 to the second amino acid residue;
the first amino acid residue and the second amino acid residue are of different types; and
the first amino acid residue and the at least one third amino acid residue are of a same type.

In some embodiments, a conjugate includes:
an antibody or antigen-binding fragment thereof includes at least a light chain, wherein the light chain includes a first amino acid residue and a second amino acid residue on its surface;
q1 number of first functional units conjugated to the light chain, wherein each of the first functional units includes a first functional moiety and a first linker residue, wherein one of the first functional unit is conjugated to the first amino acid residue on the surface of the light chain via the first linker residue; and
r number of second functional units conjugated to the light chain, wherein each of the second functional units includes a second functional moiety and a second linker residue, wherein one of the second functional unit is conjugated to the second amino acid residue on the surface of the light chain via the second linker residue.

In some embodiments of the conjugate, the antibody or antigen-binding fragment thereof further includes at least a heavy chain or heavy chain fragment thereof including at least a third amino acid residue on its surface; and q2 number of first functional units conjugated to the heavy chain or heavy chain fragment thereof, wherein the first functional unit is conjugated to the third amino acid residue on the surface of the heavy chain or heavy chain fragment thereof via the first linker residue.

In some embodiments of the conjugate, the second amino acid residue is an amino acid residue other than cysteine residue; or the second amino acid residue is selected from the group consisting of histidine residue, arginine residue, tyrosine residue, serine residue, threonine residue, lysine residue, aspartic acid residue, glutamic acid residue, and tryptophan residue.

In some embodiments of the conjugate, a molecular weight of each one of the first functional unit and the second functional unit is 0.5 to 20 kDa.

In some embodiments of the conjugate, a molecular weight of each one of the first functional unit and the second functional unit is 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20 kDa.

In some embodiments of the conjugate, the second linker residue includes a structure represented by the following formula:

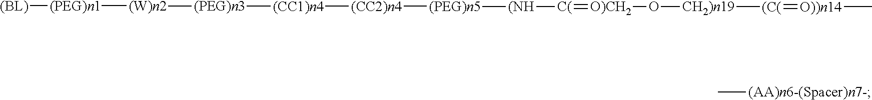

wherein,
-(PEG)n1- represents —(CH₂—CH₂—O)n1-, and n1 is an integer from 0 to 30;
W is a functional linker residue including —C(═O)—(CH₂)n8-S-(Succinimid-3-yl-N)—(CH₂)n16-, —C(═O)—(CH₂)n10-, maleimidocaproic acid, an alkyne, a 3-(pyridin-2-yldisulfanyl) propanoate (PDP) residue, an azide, an alkene, an ester, a sulfhydryl group, a hydroxyl group, a thiol, an aldehyde, a ketone, a photoreactive moieties, a glucuronide, a glucurunoside, —S—, or —S—R³—, and n2 is 0 or 1, wherein n8 is an integer from 0 to 10, n10 is an integer from 0 to 10, and n16 is an integer from 0 to 10;
-(Succinimid-3-yl-N)— has a structure represented by the following formula:

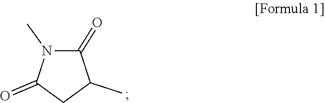

R³ is selected from the group consisting of C₁₋₆ alkyl, C₁₋₆ alkoxy, halo, halo(C₁₋₆)alkyl, hydroxy, C₁₋₆ alkylthio, C₁₋₆ alkoxycarbonyl, cyano, nitro, amino, mono- or di-C₁₋₆ alkylamino, C₁₋₆ acylamino, C₁₋₆ acyl, carboxy, C₁₋₆ alkoxycarbonyl, C₁₋₆ alkylsulfonyl, C₁₋₆ alkylsulfonamido, and phenyl or substituted phenyl;
-(PEG)n3- represents —(CH₂—CH₂—O)n3-, and n3 is an integer from 0 to 30;
CC1 is a reaction moiety of a bioorthogonal pair; CC2 is a reaction moiety of the bioorthogonal pair; and n4 is 0 or 1;
-(PEG)n5- represents —(CH₂—CH₂—O)n5-, and n5 is an integer from 0 to 30;
-(AA)n6- represents n6 amino acid residues selected from the group consisting of the basic amino acid, the neutral amino acid, the hydrophobic amino acid, the polar acidic amino acid, and any combination thereof, wherein n6 is an integer from 0 to 30;
Spacer is a self-immolative molecule connecting an oxygen or nitrogen of the second functional moiety, wherein the spacer is hemiaminal-related molecule, heterocyclic molecule, Mannich-type molecule, PABC, an aforementioned molecule substituted with a hydrophobicity-masking entity, or other spacer, and n7 is 0 or 1;
n14 is 0 or 1, n19 is 0 or 1; and
BL is a single bond or a functional linker residue including an amino acid reactive moiety, and
wherein the antibody or antigen-binding fragment thereof is connected to the terminal of BL.

In some embodiments of the conjugate, n1 is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

In some embodiments of the conjugate, n8 is 0, 1, 2, 3, 4, 5, 6, 7, 8, 9, or 10.

In some embodiments of the conjugate, n16 is 0, 1, 2, 3, 4, 5, 6, 7, 8, 9, or 10.

In some embodiments of the conjugate, n3 is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

In some embodiments of the conjugate, n5 is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

In some embodiments of the conjugate, n6 is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

In some embodiments of the conjugate, the W represents —C(═O)—(CH₂)n8-S-(Succinimid-3-yl-N)—, —C(═O)—(CH₂)n10-, —S—CH₂—CH₂—, —S—CH₂—, —S—, or —S—R³, wherein R³ is a R³ molecule residue including an alkyl group, a cycloalkyl group, or a branched alkyl group, wherein -(Succinimid-3-yl-N)— has a structure represented by the following formula:

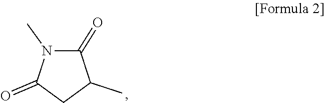

and
wherein n8 is an integer from 0 to 10, and n10 is an integer from 0 to 10.

In some embodiments of the conjugate, n10 is 0, 1, 2, 3, 4, 5, 6, 7, 8, 9, or 10.

In some embodiments of the conjugate, the BL represents a single bond, or PTDA residue having a structure represented by the following formula:

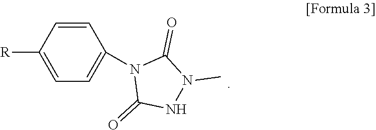

In some embodiments of the conjugate, R is —O—(CH₂)n15-; wherein n15 is an integer from 0 to 30.

In some embodiments of the conjugate, n15 is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

In some embodiments of the conjugate, -(BL)-(PEG)n1-(W)n2-(PEG)n3-(CC1)n4- has a structure represented by the following formula:

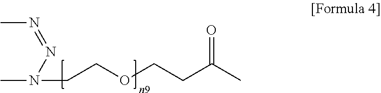

and wherein n9 is an integer from 0 to 2.

In some embodiments of the conjugate, n9 is 0, 1, or 2.

In some embodiments of the conjugate, the conjugate binds to a target antigen with a first EC50, and the antibody or antigen-binding fragment thereof binds to the target antigen with a second EC50, wherein the first EC50 is less than 100 times of the second EC50; or a first half maximal inhibitory concentration (IC50) of the first functional moiety in the first functional unit is greater than a second IC50 of the second functional moiety; or a first half maximal effective concentration (EC50) of the first functional moiety is greater than a second EC50 of the second functional moiety.

In some embodiments of the conjugate, the first EC50 is less than 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 25, 30, 35, 40, 45, 50, 60, 70, 80, 90, or 100 times of the second EC50.

In some embodiments of the conjugate, the -(AA)n6- represents n6 amino acids residues selected from the group consisting of a basic amino acid, a neutral amino acid, a hydrophobic amino acid, a polar acidic amino acid, and any combination thereof.

In some embodiments of the conjugate, the n6 amino acid residues are linked in sequence.

In some embodiments of the conjugate, the CC1, the CC2, the CC1 residue, or the CC2 residue is a bioorthogonal pair selected from the group consisting of oxime or hydrazone ligation, a Pictet or Spengler ligation, an amino benzamidoxime ligation, a Staudinger ligation, an azide-alkyne cycloaddition reaction, a strain-promoted 1,3-dipolar cycloaddition reaction, a strain-promoted cycloaddition reaction, a tetrazine-based Inverse-electron demand diels-alder reactions with strained alkenes and alkynes, a photo-induced 1,3-dipolar cycloadditions of 2,5-diaryltetrazoles and alkenes, —S—, -(Succinimid-3-yl-N)—R⁴, N₃—R⁵, or DBCO-R⁶; wherein DBCO-R⁶ is represented by the following structure:

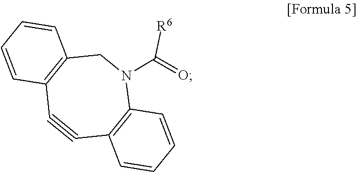

wherein -(Succinimid-3-yl-N)— has a structure represented by the following formula:

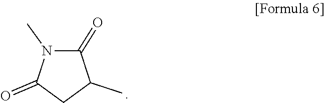

In some embodiments of the conjugate, R⁵ is a single bond or a R⁵ molecule residue including a halide phenyl group, an NHS ester, a PTAD group, a —CH₂CO2-NHS group, an aminoacetic acid-NHS ester group, a succinimidyl carbonate group, a pegylated halide phenyl group, a pegylated NHS ester, a pegylated —CH₂CO2-NHS group, an pegylated aminoacetic acid-NHS ester group, a pegylated succinimidyl carbonate group, a fluorophenyl ester group, a pegylated fluorophenyl ester group, an acid group, a pegylated acid group, a hydrazine group, or a maleimide group, and R⁶ is a R⁶ molecule residue including an alkyl group, a polymer of PEG group, a —(CH₂CH₂—O)n- group, a glycuronate group, or —(CH₂)n11-C(═O)—(NH)n12-(CH₂)n13-; and wherein n11 is an integer from 0 to 30, n12 is an integer from 0 to 30, and n13 is an integer from 0 to 30, and -(Succinimid-3-yl-N)—R⁴ is represented by one of the following structures:

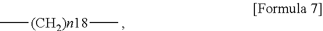

wherein n18 is an integer from 0 to 10;

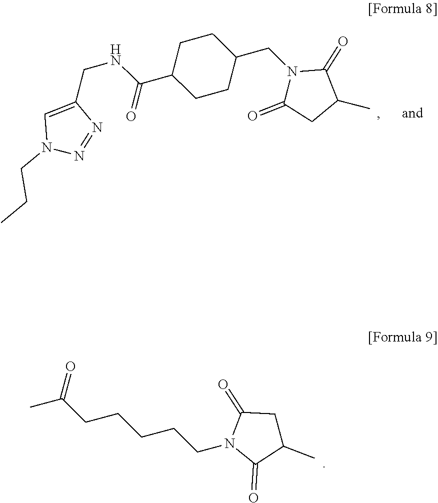

In some embodiments of the conjugate, n11 is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

In some embodiments of the conjugate, n12 is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

In some embodiments of the conjugate, n13 is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

In some embodiments of the conjugate, n18 is 0, 1, 2, 3, 4, 5, 6, 7, 8, 9, or 10.

In some embodiments of the conjugate, Spacer is represented by one of the following structures:

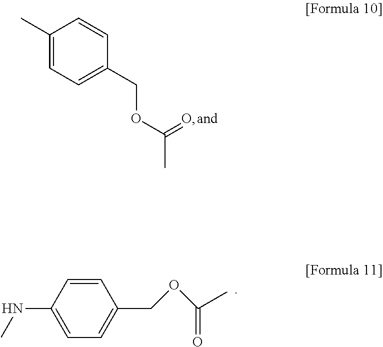

In some embodiments of the conjugate, the first amino acid residue, the second amino acid residue, and the third amino acid residue are native amino acid residues.

In some embodiments of the conjugate, the second functional unit is represented by one of the following structures:

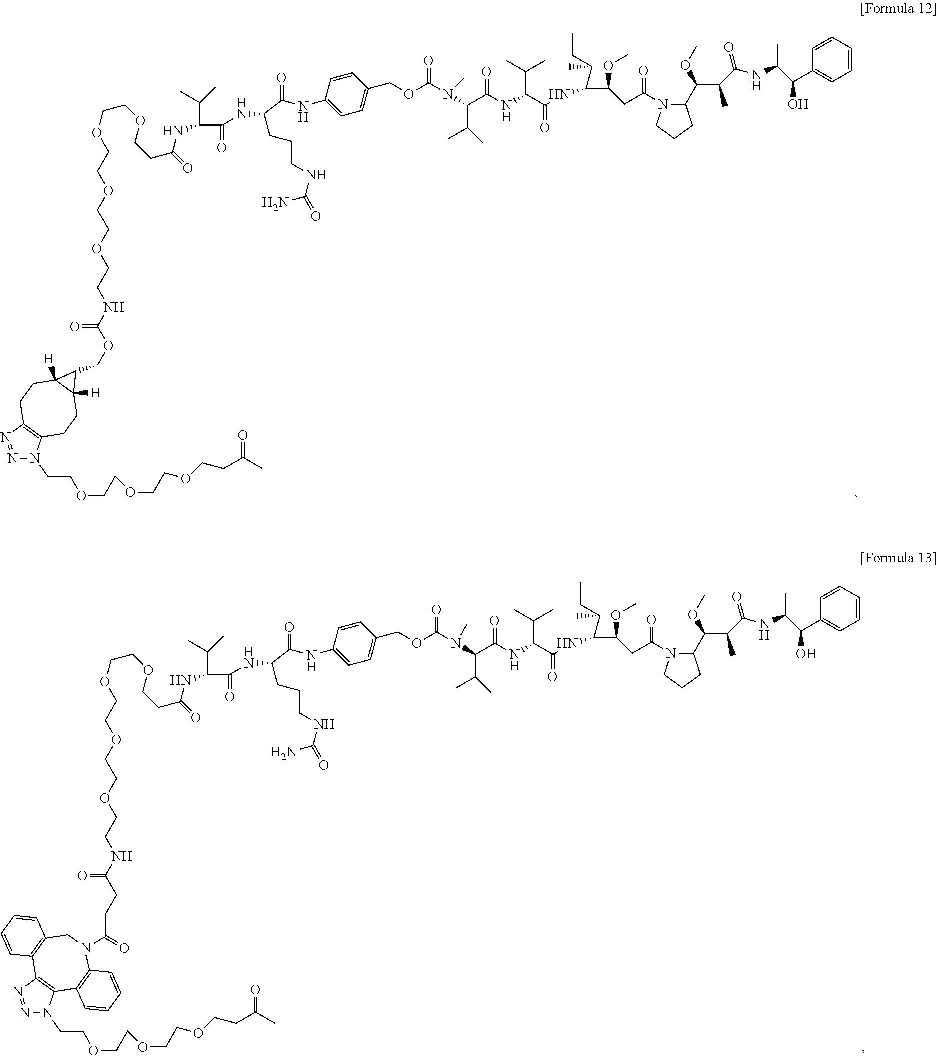

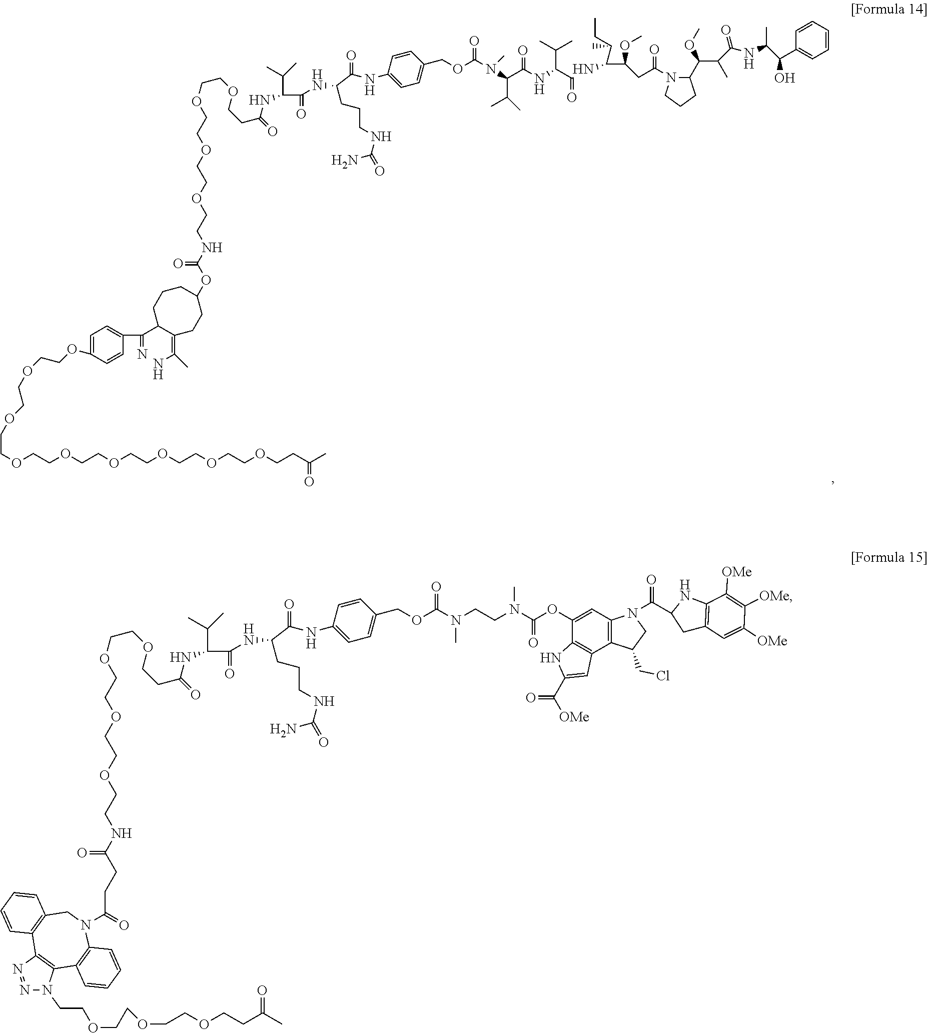

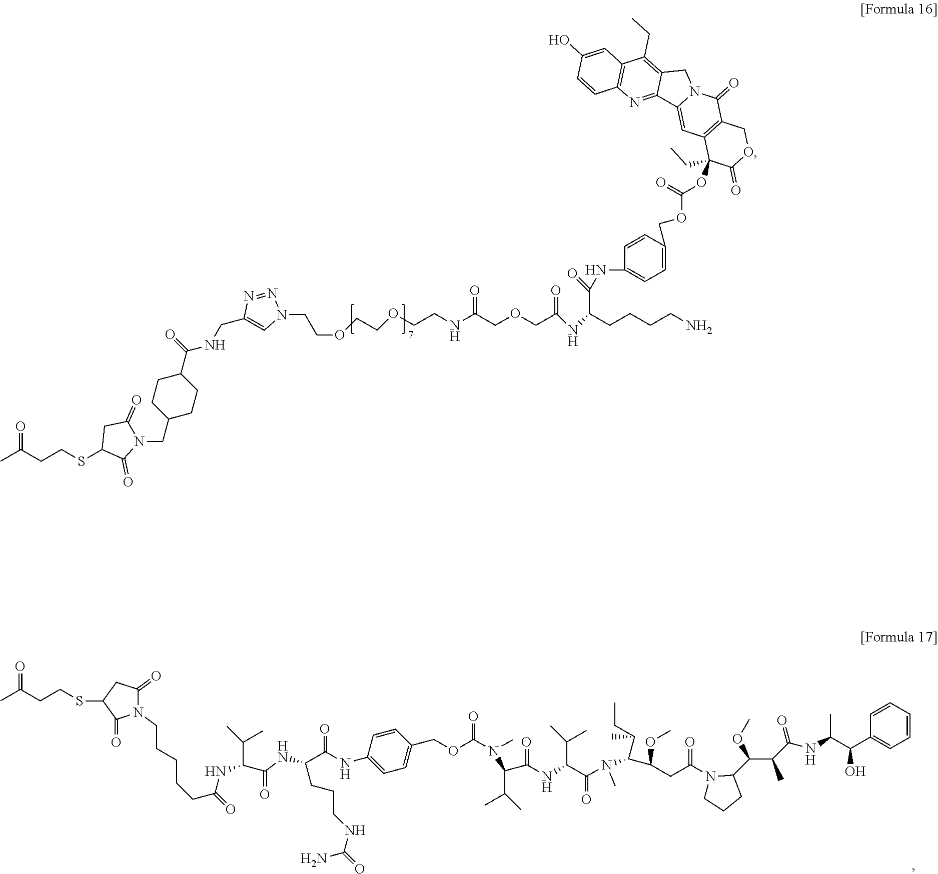

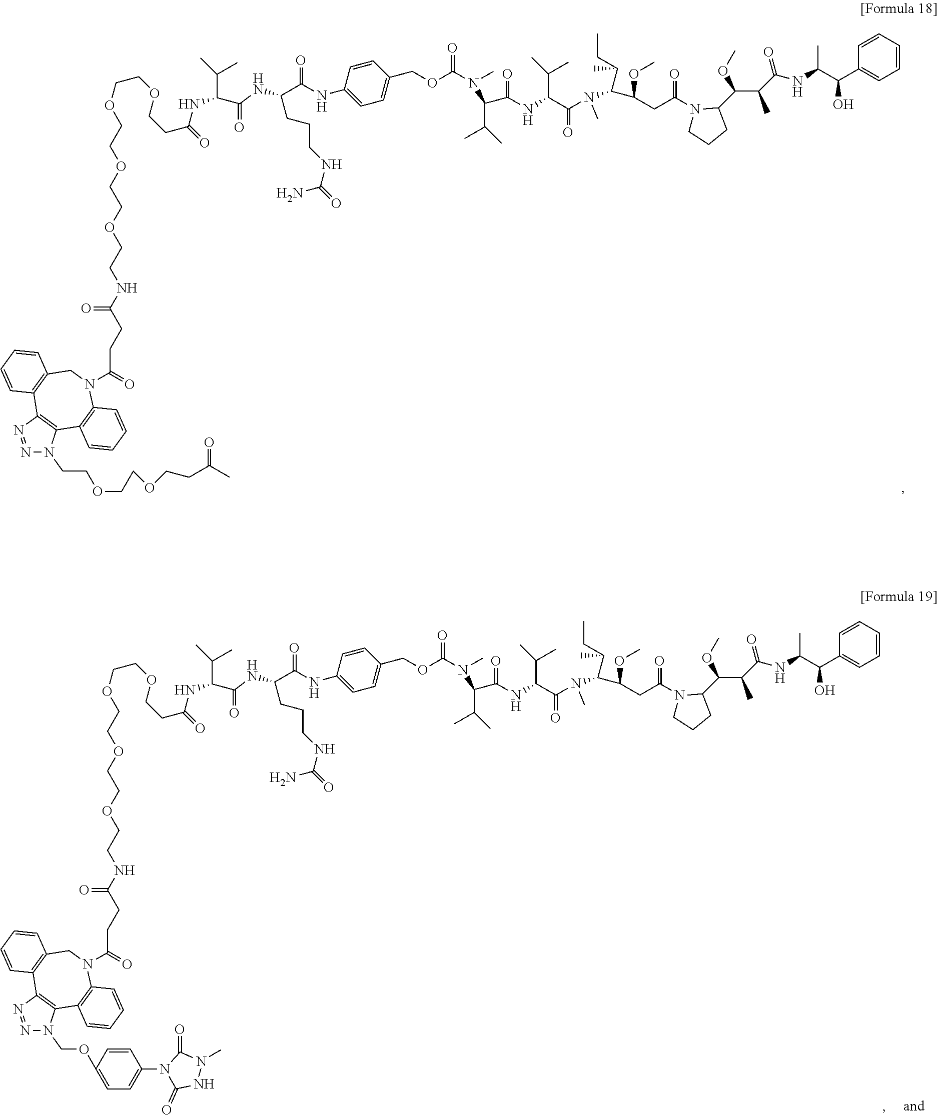

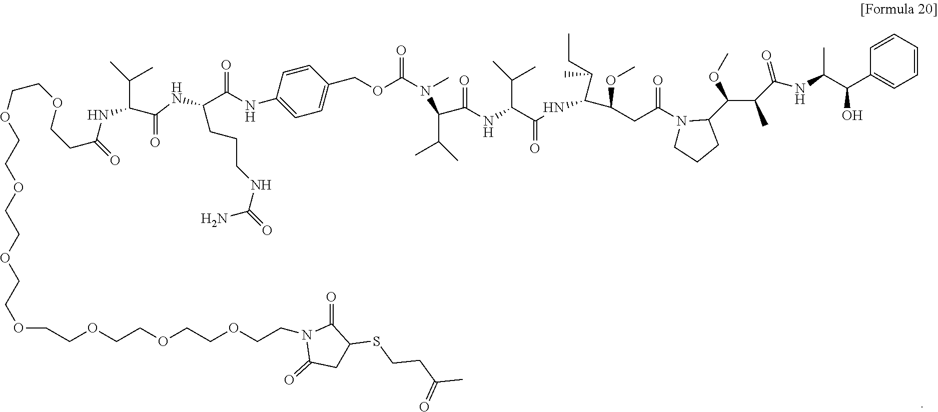 [SMILES: CC[C@H](C)[C@@H](NC(=O)[C@H](NC(=O)[C@@H](C(C)C)N(C)C(=O)OCc1ccc(NC(=O)[C@H](CCCNC(N)=O)NC(=O)[C@H](NC(=O)CCOCCOCCOCCOCCOCCOCCOCCOCCN2C(=O)CC(SCCC(C)=O)C2=O)C(C)C)cc1)C(C)C)[C@H](CC(=O)N1CCCC1[C@@H](OC)[C@H](C)C(=O)N[C@@H](C)[C@H](O)c1ccccc1)OC]

In some embodiments of the conjugate, the first amino acid residue and the third amino acid residue are cysteine residue.

In some embodiments of the conjugate, the antibody or antigen-binding fragment thereof is an IgG1 antibody, IgM antibody, IgD antibody, IgG antibody, IgA antibody, or IgE antibody.

In some embodiments of the conjugate, the light chain is kappa light chain, lambda light chain, or a combination thereof.

In some embodiments of the conjugate, the first linker residue includes a maleimide residue, and the first functional unit is conjugated to the first amino acid residue or the third amino acid residue via a covalent interaction between the first amino acid residue and the maleimide residue.

In some embodiments of the conjugate, the first linker residue includes a structure represented by the following formula.

-TR-(Y)m1-(PEG)m2-(Z)m3-(AA)m4-(Spacer)m5-;
wherein,
TR is a residue including a sulfhydryl-reactive group;
Y is a Y molecule residue, —(CH₂)m6-(C═O)—, —(CH₂)m7-(C═O)—NH—(CH₂)m8-, or other group, and the Y molecule residue includes an alkyl group, a cycloalkyl group, a branched alkyl group, a glucuronide, a disulfide, an ester, a hydrazone, a thioether or any combination thereof, wherein m1 is 0 or 1, m6 is an integer from 0 to 10, m7 is an integer from 0 to 10, and m8 is an integer from 0 to 10;
-(PEG)m2- represents —(CH₂—CH₂—O)m2-, and m2 is an integer from 0 to 30;
Z is an Z molecule residue including alkyl group, a pH-sensitive structure, a hydrazone, a disulfide, a glucuronide, a pyrophosphate, —(C═O)—, or other structure, and m3 is an integer from 0 to 10;
-(AA)m4- represents m4 amino acid residues selected from the group consisting of the basic amino acid, the neutral amino acid, the hydrophobic amino acid, the polar acidic amino acid, and any combination thereof, and m4 is an integer from 0 to 30;
Spacer is a self-immolative molecule connecting an oxygen or nitrogen of the second functional moiety, wherein the spacer is hemiaminal-related molecule, heterocyclic molecule, Mannich-type molecule, PABC, an aforementioned molecule substituted with a hydrophobicity-masking entity, or other spacer, and m5 is 0 or 1, and
wherein the antibody or antigen-binding fragment thereof is connected to the terminal of TR.

In some embodiments of the conjugate, m2 is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

In some embodiments of the conjugate, m4 is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

In some embodiments of the conjugate, m6 is 0, 1, 2, 3, 4, 5, 6, 7, 8, 9, or 10.

In some embodiments of the conjugate, m7 is 0, 1, 2, 3, 4, 5, 6, 7, 8, 9, or 10.

In some embodiments of the conjugate, m8 is 0, 1, 2, 3, 4, 5, 6, 7, 8, 9, or 10.

In some embodiments of the conjugate, TR is a maleimide, a maleimide residue, or -(Succinimid-3-yl-N)—.

In some embodiments of the conjugate, Y is represented by one of the following structures:

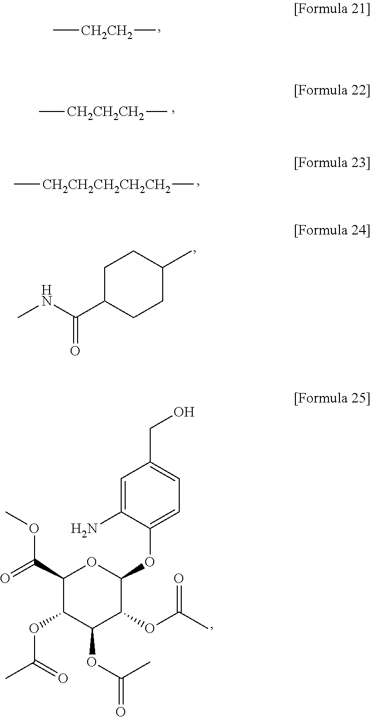

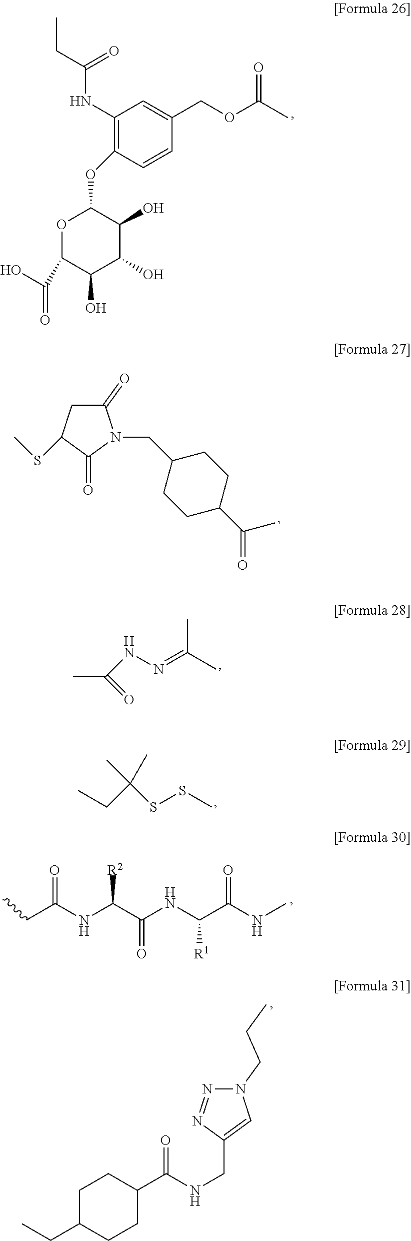

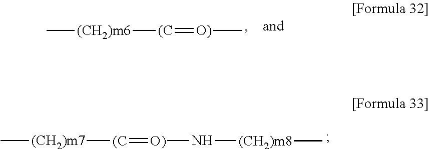

wherein R¹ and R² are each independently selected from the side chains of naturally occurring α-amino acids and non-naturally occurring analogs thereof, including hydrogen, C₁₋₆ straight or branched alkyl, hydroxy-substituted C₁₋₆ alkyl, thio-substituted C₁₋₆ alkyl, carboxy-substituted C₁₋₆ alkyl, amino-substituted C₁₋₆ alkyl, arylmethyl, heteroarylmethyl, and guanidino-, imidazolyl-, or indolyl-containing side chains.

In some embodiments of the conjugate, wherein Z is —(C═O)— or represented by one of the following structures:

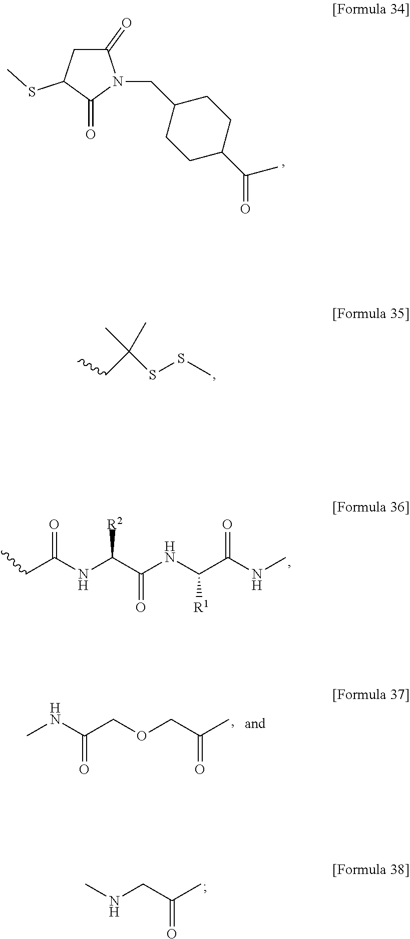

wherein R¹ and R² are each independently selected from the side chains of naturally occurring α-amino acids and non-naturally occurring analogs thereof, including hydrogen, C₁₋₆ straight or branched alkyl, hydroxy-substituted C₁₋₆ alkyl, thio-substituted C₁₋₆ alkyl, carboxy-substituted C₁₋₆ alkyl, amino-substituted C₁₋₆ alkyl, arylmethyl, heteroarylmethyl, and guanidino-, imidazolyl-, or indolyl-containing side chains.

In some embodiments of the conjugate, the -(AA)m4- represents m4 amino acid residues selected from the group consisting of a basic amino acid, a neutral amino acid, a hydrophobic amino acid, a polar acidic amino acid, and any combination thereof.

In some embodiments of the conjugate, the m4 amino acid residues are linked in sequence.

In some embodiments of the conjugate, the Spacer is represented by one of the following structures:

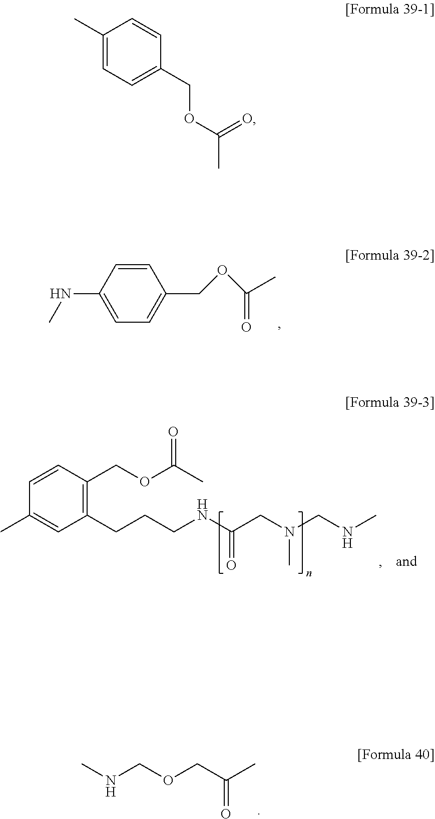

In some embodiments of the conjugate, the first functional unit is represented by one of the following structures:

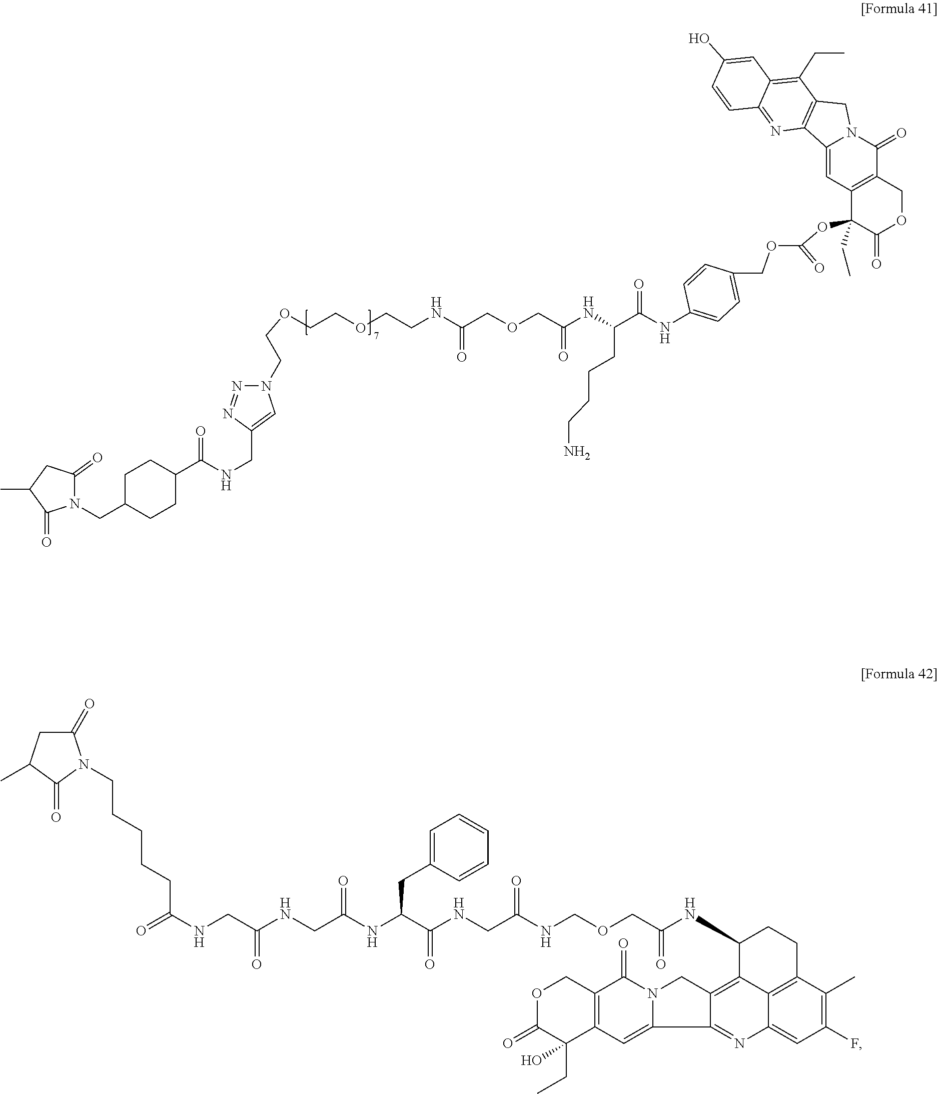

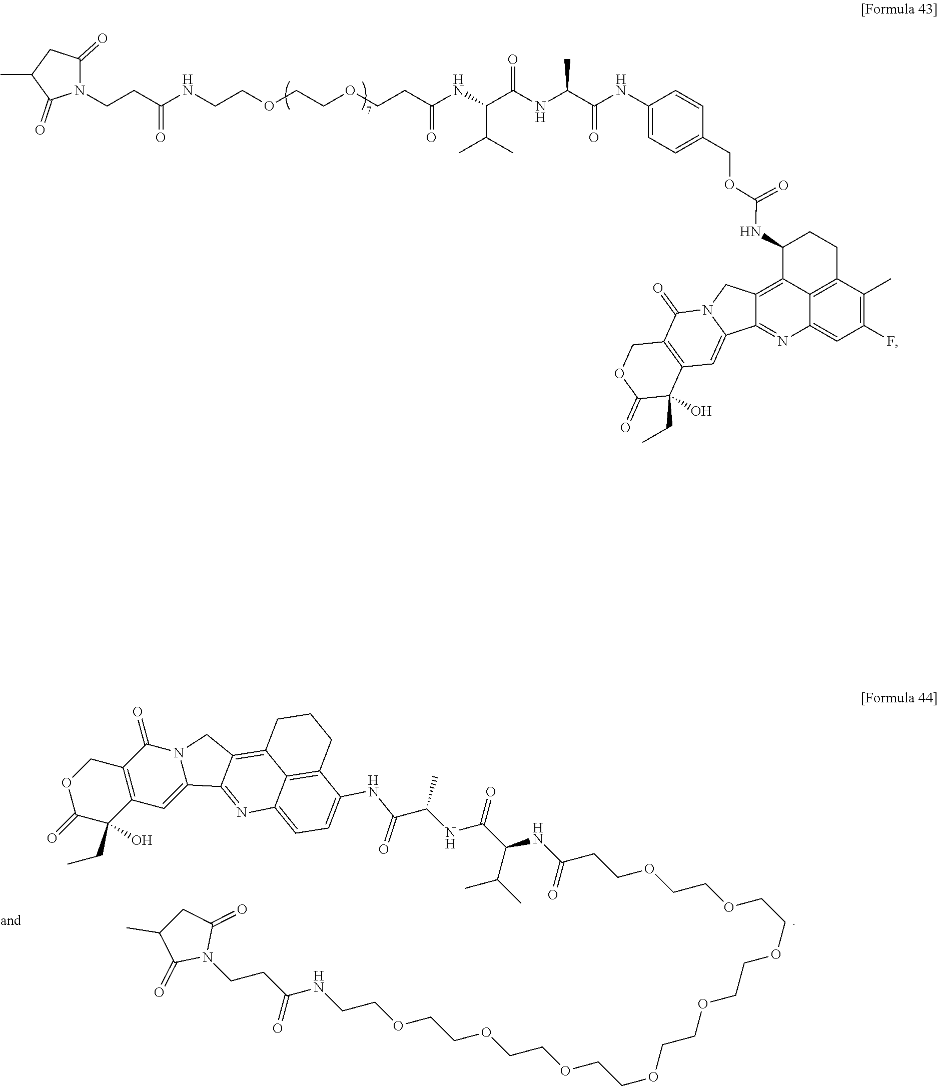

In some embodiments of the conjugate, q1 is an integer from 1 to 2, and r is an integer from 1 to 2.

In some embodiments of the conjugate, the sum of q1 and q2 is from 1 to 8.

In some embodiments of the conjugate, the conjugate is represented by the following structure of formula:

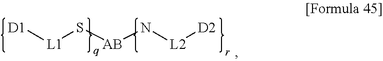

wherein,
AB is the antibody or antigen-binding fragment thereof;
S is a sulfur in the first amino acid residue or the third amino acid residue;
N is a nitrogen in the second amino acid residue;
D1 is the first functional moiety;
D2 is the second functional moiety;
L1 is the first linker residue connecting the first functional moiety to the antibody or antigen-binding fragment thereof;
L2 is the second linker residue connecting the second functional moiety to the antibody or antigen-binding fragment thereof, and
q is the sum of q1 and q2.

In some embodiments of the conjugate, q is from 1 to 8, and r is from 1 to 2; or wherein q is 8, and r is 2.

In some embodiments of the conjugate, q is 1, 2, 3, 4, 5, 6, 7, or 8. In some embodiments of the conjugate, r is 1 or 2.

In some embodiments of the conjugate, the first amino acid residue or the third amino acid residue is a cysteine residue, and the sulfur is the sulfur in the side chain of the cysteine residue.

In some embodiments of the conjugate, the second amino acid residue is a lysine residue, and the nitrogen is the nitrogen in the side chain of the lysine residue.

In some embodiments of the conjugate, the antibody or antigen-binding fragment thereof is an antibody, or wherein the antibody or antigen-binding fragment thereof is a synthetic antibody, an isolated antibody, native antibody, an engineered antibody, a chimeric antibody, a bispecific antibody, a cell engager antibody, a single-chain variable fragment (scFv), Fab fragment, Fab′ fragment, F(ab′)2 fragment, or Fv fragment, or other antigen-binding fragment.

In some embodiments of the conjugate, at least one of the first functional moiety and the second functional moiety is individually an antitumor compound.

In some embodiments of the conjugate, at least one of the first functional moiety and the second functional moiety is individually an active agent, a therapeutic moiety, a fluorophore, a nanoparticle, a spin label, a radioactive moiety, a photocaged moiety, an enzyme, or an imaging agent.

In some embodiments of the conjugate, at least one of the first functional moiety and the second functional moiety is individually an anti-neoplastic agent, an immune-modulating agent, an anti-cancer agent, a chemotherapeutic drug, a DNA-modifying agent, DNA-damaging agent, a tubulin inhibitor, a proteolysis targeting chimera (PROTAC), or a photoimmunotherapy drug.

In some embodiments of the conjugate, the first functional moiety and the second functional moiety in the conjugate manifest synergy in treating a disease.

In some embodiments of the conjugate, the disease is a cancer, a tumor, an autoimmune disease, a neuronal disease, a hematopoietic cell-related disease, a metabolic syndrome, a pathogenic disease, a viral infectious disease, a fungal infectious disease, a protozoan infectious disease, or a bacterial infectious disease.

In some embodiments of the conjugate, the conjugate includes:
2, 4, 6, or 8 first functional units; and
1 or 2 second functional unit(s).

In some embodiments of the conjugate, in the conjugate, the ratio of the first functional units to the second functional units is 1:1, 2:1, 3:1, 4:1, 6:1, or 8:1.

In some embodiments of the conjugate, the conjugate further includes d number of third functional unit(s) conjugated to the light chain, wherein each of the third functional units includes a third functional moiety and a third linker residue; and one of the third functional unit is conjugated to a fourth amino acid residue on the surface of the light chain via the third linker residue, and the fourth amino acid residue is different from the first, second, and third amino acid residues.

In some embodiments of the conjugation provided herein, the fourth amino acid residue is a tyrosine residue.

In some embodiments of the conjugate, the second linker residue includes a structure represented by the following formula:

-(BL)-(PEG)n1-(W)n2-(PEG)n3-(CC1)n4-;
wherein,
-(PEG)n1- represents —(CH₂—CH₂—O)n1-, and n1 is an integer from 0 to 30;
W is a functional linker residue including —C(═O)—(CH₂)n8-S-(Succinimid-3-yl-N)—(CH₂)n16-, —C(═O)—(CH₂)n10-, maleimidocaproic acid, an alkyne, a 3-(pyridin-2-yldisulfanyl) propanoate (PDP) residue, an azide, an alkene, an ester, a sulfhydryl group, a hydroxyl group, a thiol, an aldehyde, a ketone, a photoreactive moiety, a glucuronide, a glucurunoside, —S—, or —S—R³—, and n2 is 0 or 1, wherein n8 is an integer from 0 to 10, n10 is an integer from 0 to 10, and n16 is an integer from 0 to 10;
-(Succinimid-3-yl-N)— is represented by the following formula:

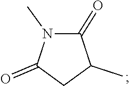

R³ is selected from the group consisting of C₁₋₆ alkyl, C₁₋₆ alkoxy, halo, halo(C₁₋₆)alkyl, hydroxy, C₁₋₆ alkylthio, C₁₋₆ alkoxycarbonyl, cyano, nitro, amino, mono- or di-C₁₋₆ alkylamino, C₁₋₆ acylamino, C₁₋₆ acyl, carboxy, C₁₋₆ alkoxycarbonyl, C₁₋₆ alkylsulfonyl, C₁₋₆ alkylsulfonamido, and phenyl or substituted phenyl;
-(PEG)n3- represents —(CH₂—CH₂—O)n3-, and n3 is an integer from 0 to 30;
CC1 is a reaction moiety of a bioorthogonal pair; and
BL is a single bond or a functional linker residue including amino acid reactive moiety, and wherein the antibody or antigen-binding fragment thereof is connected to the terminal of BL; and wherein the conjugate is resistant to protein aggregation.

In some embodiments, a pharmaceutical composition includes the conjugate, or a salt thereof as an active component, and a pharmaceutically acceptable carrier.

In some embodiments, optionally, the pharmaceutical composition is resistant to protein aggregation, optionally wherein >90% of the protein in the pharmaceutical composition is in a non-aggregated state over the formulation's shelf life.

In some embodiments, a method of treating a disease or disorder in a subject in need thereof includes administering to the subject an effective amount of the conjugate, or a salt thereof, wherein the disease/disorder is a tumor, a cancer, an autoimmune disease, a neuronal disease, a hematopoietic cell-related disease, a metabolic syndrome, a pathogenic disease, a viral infectious disease, a fungal infectious disease, a protozoan infectious disease, a bacterial infectious disease, or other disease/disorder.

In some embodiments of the treating method, the subject is a human.

In some embodiments, a method of manufacturing the conjugate includes:
providing an antibody or antigen-binding fragment thereof, wherein the antibody or antigen-binding fragment thereof comprises a light chain having a first amino acid residue and a second amino acid residue, and a heavy chain having at least one third amino acid residue; and the first amino acid residue and the second amino acid residue are of different types, and the first amino acid residue and the at least one third amino acid residue are of a same type;
conjugating at least one first functional unit to the first amino acid residue and the at least one third amino acid residue, wherein each the first functional unit comprises a sulfhydryl-reactive group and has a formula -L1-D1, by the steps of:
   reducing the antibody or antigen-binding fragment thereof to expose free thiol groups on the first amino acid residue and the at least one third amino acid residue; and
   reacting the first functional unit with the free thiol groups exposed in the reducing step to form a covalent bond between each free thiol group and the sulfhydryl-reactive group of the first functional unit; and
conjugating a second functional unit having a formula -L2-D2 to the second amino acid residue by the steps of:
   reacting an AAR linker comprising an AAR group with the antibody or antigen-binding fragment thereof to produce an intermediate conjugate comprising a linker residue covalently bonded to the second amino acid residue, wherein the intermediate conjugate does not comprise the AAR group; and
   contacting a molecule comprising a functional moiety with the intermediate conjugate of the reacting step to form a covalent linkage between the molecule and the linker residue of the intermediate conjugate via the reaction of reaction moieties of a bioorthogonal pair, thereby producing the second functional unit represented by the formula -L2-D2,
wherein,
   L1 is a first linker residue, L2 is a second linker residue, and D1 and D2 are each independently a functional moiety.

In some embodiments, a method of manufacturing the conjugate includes:
procedure for linking an antibody or antigen-binding fragment thereof with (A) a first functional unit and (B) a second functional unit;
wherein, the procedure (A) for linking the antibody or antigen-binding fragment thereof with the first functional unit includes the following steps (a1) to (a3):
(a1) providing the antibody or antigen-binding fragment thereof, wherein the antibody or antigen-binding fragment thereof includes:
   a light chain including on its surface a first amino acid residue and a second amino acid residue, and the first light chain is a kappa light chain; and
   heavy chains or heavy chain fragments; wherein each of the heavy chains or heavy chain fragments includes on its surface at least a third amino acid residue;
(a2) in a first conjugation buffer, contacting the antibody or antigen-binding fragment thereof with a first reducing agent, to expose first free thiols in the first amino acid residue;
(a3) in the first conjugation buffer, linking the antibody or antigen-binding fragment thereof with a first functional unit via a first covalent bond between the first amino acid residue and a TR residue of the first functional unit, wherein the TR residue includes a sulfhydryl-reactive group; and
wherein, the procedure (B) for linking the antibody or antigen-binding fragment thereof with the second functional unit includes the following steps (b1) to (b2):
(b1) in a second conjugation buffer, contacting the antibody or antigen-binding fragment thereof with an AAR linker, to generate a linker residue including a BL residue covalently bonded to the second amino acid residue;
(b2) in a third conjugation buffer, reacting with a molecule including a second functional moiety, to link the second amino acid residue and the molecule including the second functional moiety via the reaction between the linker residue and the molecule including the second functional moiety.

In some embodiments of the manufacturing method, the antibody or antigen-binding fragment thereof is an IgG 1 antibody.

In some embodiments of the manufacturing method, in step (a2), the first reducing agent is contacted with the 4 interchain disulfide bonds of the IgG 1 antibody, to expose 8 first free thiols in the 8 cysteine residues.

In some embodiments of the manufacturing method, the antibody or antigen-binding fragment thereof is an IgG antibody, IgD antibody, IgA antibody, or IgE antibody.

In some embodiments of the manufacturing method, in step (a2), the first reducing agent is contacted with the 4 interchain disulfide bonds of the IgG antibody, IgD antibody, IgA antibody, or IgE antibody, to expose 8 first free thiols in the 8 cysteine residues.

In some embodiments of the manufacturing method, the antibody or antigen-binding fragment thereof is an IgA antibody.

In some embodiments of the manufacturing method, in step (a2), the first reducing agent is contacted with the 8 interchain disulfide bonds of the IgA antibody, to expose 8 first free thiols in the 16 cysteine residues.

In some embodiments of the manufacturing method, the antibody or antigen-binding fragment thereof is an IgM antibody.

In some embodiments of the manufacturing method, in step (a2), the first reducing agent is contacted with the 20 interchain disulfide bonds of the IgM antibody, to expose 8 first free thiols in the 40 cysteine residues.

In some embodiments of the method, the kappa light chain includes a lysine residue corresponding to a position 188 (Lys188) or 190 (Lys190), or wherein the antibody or antigen-binding fragment thereof is an immunoglobulin G (IgG) antibody.

In some embodiments of the manufacturing method, the AAR linker includes a structure represented by the following formula:

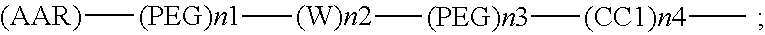

wherein,
-(PEG)n1- represents —(CH₂—CH₂—O)n1-, and n1 is an integer from 0 to 30;
W is a functional linker residue including —C(═O)—(CH₂)n8-S-(Succinimid-3-yl-N)—, —C(═O)—(CH₂)n10-, maleimidocaproic acid, an alkyne, a 3-(pyridin-2-yldisulfanyl) propanoate (PDP) residue, an azide, an alkene, an ester, a sulfhydryl group, a hydroxyl group, a thiol, an aldehyde, a ketone, a photoreactive moieties, —S—, or —S—R³—, and n2 is 0 or 1, wherein n8 is an integer from 0 to 10, and n10 is an integer from 0 to 10;
-(Succinimid-3-yl-N)— has a structure represented by the following formula:

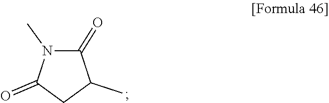

R³ is selected from the group consisting of C₁₋₆ alkyl, C₁₋₆ alkoxy, halo, halo(C₁₋₆)alkyl, hydroxy, C₁₋₆ alkylthio, C₁₋₆ alkoxycarbonyl, cyano, nitro, amino, mono- or di-C₁₋₆ alkylamino, C₁₋₆ acylamino, C₁₋₆ acyl, carboxy, C₁₋₆ alkoxycarbonyl, C₁₋₆ alkylsulfonyl, C₁₋₆ alkylsulfonamido, and phenyl or substituted phenyl;
-(PEG)n3- represents —(CH₂—CH₂—O)n3-, and n3 is an integer from 0 to 30;
CC1 is a reaction moiety of a bioorthogonal pair; and
AAR has a structure represented by one of the following formulae:

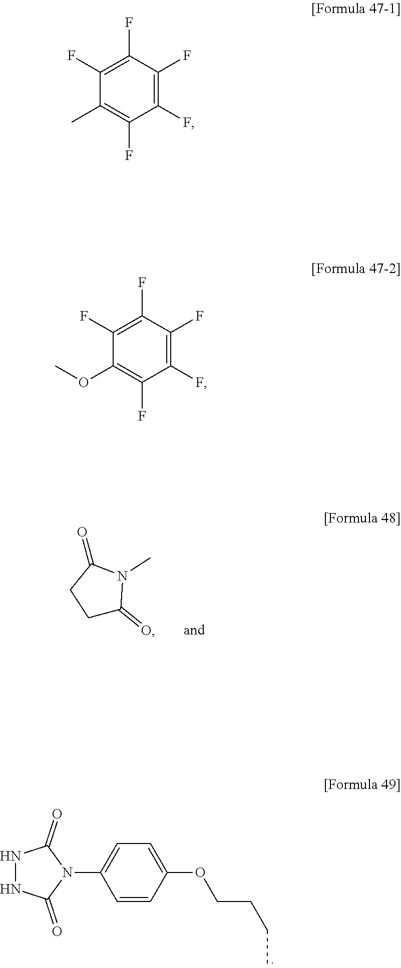

In some embodiments of the manufacturing method, the molecule including the second functional moiety is represented by the following structure:

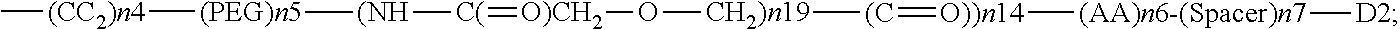

wherein,
   CC2 is a reaction moiety of the bioorthogonal pair; and n4 is 0 or 1;
   -(PEG)n5- represents —(CH₂—CH₂—O)n5-, and n5 is an integer from 0 to 30;
   -(AA)n6- represents n6 amino acid residues selected from the group consisting of the basic amino acid, the neutral amino acid, the hydrophobic amino acid, the polar acidic amino acid, or any combination thereof, wherein n6 is an integer from 0 to 30;
   Spacer is a self-immolative molecule connecting an oxygen or nitrogen of the second functional moiety, wherein the spacer is hemiaminal-related molecule, heterocyclic molecule, Mannich-type molecule, PABC, an aforementioned molecule substituted with a hydrophobicity-masking entity, or other spacer, and n7 is 0 or 1;
   n14 is 0 or 1, n19 is 0 or 1; and
   D2 is a second functional moiety.

In some embodiments of the manufacturing method, the n6 amino acid residues are linked in sequence.

In some embodiments of the manufacturing method, the first conjugation buffer is a phosphate buffer, an acetate buffer, a Tris buffer, a citrate buffer, a sodium chloride solution, an EDTA buffer, or an amino acid-containing buffer.

In some embodiments of the method, the AAR linker is a PFP-based linker.

In some embodiments of the manufacturing method, the PFP-based linker includes a disulfide bond or an azide functional group.

In some embodiments of the method, the PFP-based linker is a propargyl-SS-PFP ester, a 2,3,4,5,6-pentafluorophenyl 4-(pyridin-2-yldisulfanyl) butanoate (PDB-PFP), a 2,3,4,5,6-pentafluorophenyl 3-(pyridin-2-yldisulfanyl)propanoate (PDP-PFP), a perfluorophenyl 3-(pyridin-2-yldisulfanyl)propanoate, perfluorophenyl 4-methyl-4-(pyridine-2-yldisulfanyl)pentanoate, or a perfluorophenyl 4-(pyridin-2-yldisulfanyl)butanoate.

In some embodiments of the manufacturing method, the second conjugation buffer is a phosphate buffer, an acetate buffer, a Tris buffer, a citrate buffer, a sodium chloride solution, an EDTA buffer, or an amino acid-containing buffer.

In some embodiments of the manufacturing method, the third conjugation buffer is a phosphate buffer, an acetate buffer, a Tris buffer, a citrate buffer, a sodium chloride solution, an EDTA buffer, or an amino acid-containing buffer.

In some embodiments of the manufacturing method, a first half maximal inhibitory concentration (IC50) of the first functional moiety in the first functional unit is greater than a second IC50 of the second functional moiety; or a first half maximal effective concentration (EC50) of the first functional moiety is greater than a second EC50 of the second functional moiety; or a first median lethal dose (LD50) of the second functional moiety is greater than a second LD50 of the first functional moiety; or a first median toxic dose (TD50) of the second functional moiety is greater than a second TD50 of the first functional moiety.

In some embodiments of the manufacturing method, the step (b2) includes:
(b21) in the second conjugation buffer, reducing a disulfide bond in the linker residue with a second reducing agent, to expose second free thiols; and
(b22) in a third conjugation buffer, reacting the second free thiols with a sulfhydryl group, an acrylate, an alkyl halide, an aziridine, a maleimidocaproyl group, a vinyl sulfone, a pyridyl disulfide, or other electrophile in a molecule including the second functional moiety, to link the second amino acid residue and the molecule including the second functional moiety via the reaction between the second free thiol and the molecule including the second functional moiety.

In some embodiments of the manufacturing method, the first reducing agent and/or the second reducing agent is beta-mercaptoethanol (BME), dithiothreitol (DTT), tris(2-carboxyethyl)phosphine (TCEP), 2-Mercaptoethylamine (2-MEA), or thiol-based reducing agents.

In some embodiments of the manufacturing method, a molecular weight of each one of the first functional unit and the second functional unit is 0.5 to 20 kDa.

In some embodiments of the manufacturing method, the molecular weight of each one of the first functional unit and the second functional unit is 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20 kDa.

In some embodiments of the manufacturing method, (AAR)-(PEG)n1-(W)n2-(PEG)n3-(CC1)n4- has a structure represented by the following formula:

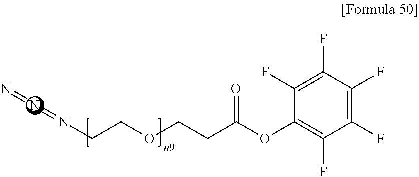

and wherein n9 is an integer from 0 to 2.

In some embodiments of the manufacturing method, n9 is 0, 1, or 2.

In some embodiments of the manufacturing method, CC1 has a structure represented by one of the following formula:

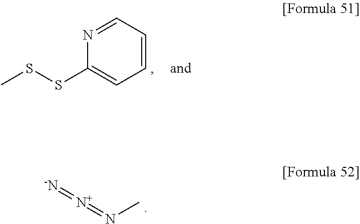

In some embodiments of the manufacturing method, the AAR linker has a structure represented by one of the following formula:

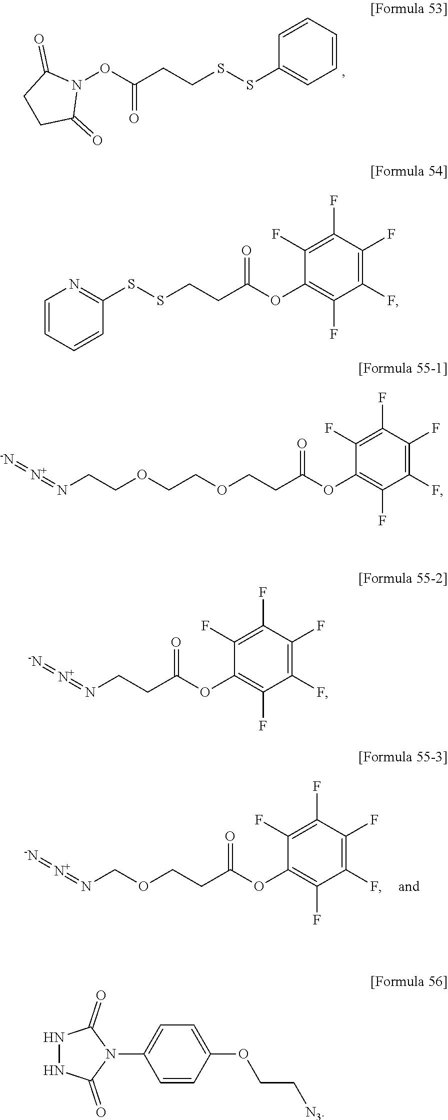

In some embodiment, a method of reducing aggregation during the preparation of a dual-payload antibody drug conjugate includes:
procedure for linking an antibody or antigen-binding fragment thereof with (A) a first functional unit and (B) a second functional unit;
wherein, the procedure (A) for linking the antibody or antigen-binding fragment thereof with the first functional unit includes the following steps (a1) to (a3):
(a1) providing the antibody or antigen-binding fragment thereof, wherein the antibody or antigen-binding fragment thereof includes:
   at least one light chain including on its surface a first amino acid residue and a second amino acid residue, and the light chain is a kappa light chain; and
   heavy chains or heavy chain fragments; wherein each of the heavy chains or heavy chain fragments includes on its surface at least a third amino acid residue;
(a2) in a first conjugation buffer, contacting the antibody or antigen-binding fragment thereof with a first reducing agent, to expose first free thiols in the first amino acid residues;
(a3) in the first conjugation buffer, linking the antibody or antigen-binding fragment thereof with a first functional unit via a first covalent bond between the first amino acid residue and a TR residue of the first functional unit, wherein the TR residue includes a sulfhydryl-reactive group; and
wherein, the procedure (B) for linking the antibody or antigen-binding fragment thereof with the second functional unit includes the following steps (b1) to (b2):
(b1) in a second conjugation buffer, contacting the antibody or antigen-binding fragment thereof with an AAR linker, to generate a linker residue including a BL residue which covalently bonded to the second amino acid residue; and
(b2) in a third conjugation buffer, reacting with a molecule including a second functional moiety, to link the second amino acid residue and the molecule including the second functional moiety via the reaction between the linker residue and the molecule including the second functional moiety.

In some embodiments, a conjugate includes:
an antibody or antigen binding fragment thereof, including:
   a first light chain and a second light chain; wherein each of the light chains includes on its surface a first amino acid residue and a second amino acid residue; and
   heavy chains or heavy chain fragments; wherein each of the heavy chains or heavy chain fragments includes on its surface at least a third amino acid;
q number of first functional unit(s), each including a first functional moiety and a first linker residue, and the first functional unit is conjugated to the first amino acid residue or the third amino acid residue via the first linker residue; wherein one of the first functional unit is conjugated to the first amino acid residue on the surface of the first light chain; and
r number of second functional unit(s), each including a second functional moiety and a second linker residue, and the second functional unit is conjugated to the second amino acid residue via the second linker residue; wherein one of the second functional unit is conjugated to the second amino acid residue on the surface of the first light chain.

In some embodiments of the conjugate, q is from 1 to 8, and r is from 1 to 2; or wherein q is 8, and r is 2.

In some embodiments of the conjugate, the conjugate further includes d number of third functional unit(s), each including a third functional moiety and a third linker residue; wherein one of the third functional unit is conjugated to a fourth amino acid residue on the surface of the first light chain, and the fourth amino acid residue is different from the first, second, and third amino acid residues.

In some embodiments of the conjugate, the fourth amino acid residue is a tyrosine residue.

### BRIEF DESCRIPTION OF THE DRAWING

FIG. 1 schematically shows the structure of an antibody molecule (monomer).

FIGS. 2A to 2D schematically depict the structure of antibody conjugates according to some embodiments of the present disclosure. FIG. 2A schematically shows the structure of an antibody conjugate including two light chains and two heavy chains, wherein one of the light chain is covalently linked to two different functional units (L1-D1 and L2-D2). It should be noted that the number of heavy and light chains depicted in the figures is merely illustrative of a conventional antibody format and should not be construed as limiting the scope of the present application to such antibodies. For example, a light-chain fragment such as a Fab or Fv fragment may also be encompassed within the scope of the present application. FIG. 2B schematically shows the structure of an antibody conjugate including two light chains and two heavy chains, wherein each one of the light chains is covalently linked to two different functional units (L1-D1 and L2-D2), and each one of the heavy chains is covalently linked to three functional units (L1-D1), and therefore there are ten functional units conjugated to an antibody (if D1 and D2 are drugs, the drug antibody ratio (DAR value) is 10; refer to DAR10 ADC). FIG. 2C schematically shows the structure of an antibody conjugate including two light chains and two heavy chains, wherein one of the light chain is covalently linked to three different functional units (L1-D1, L2-D2, and L3-D3). FIG. 2D schematically shows the structure of an antibody conjugate including two light chains and two heavy chains, wherein each one of the light chains is covalently linked to three different functional units (L1-D1, L2-D2, and L3-D3), and each one of the heavy chains is covalently linked to three functional units (L1-D1), and therefore there are 12 functional units conjugated to an antibody (if D1, D2, and D3 are drugs, the drug antibody ratio (DAR value) is 12; refer to DAR12 ADC).

FIG. 3 illustrates an example process of obtaining a DAR10 ADC via procedure (A) conjugating 8 numbers of the first functional moieties (govitecan) and procedure (B) conjugating 2 numbers of the second functional moiety (govitecan) to an antibody (Atezolizumab), wherein the first functional moieties are linked to the cysteine residue on the surface of the light chain and on the surface of the heavy chain of the antibody via the first linker residue, and the second functional moieties are linked to the lysine residue on the surface of the light chain of the antibody via the second linker residue.

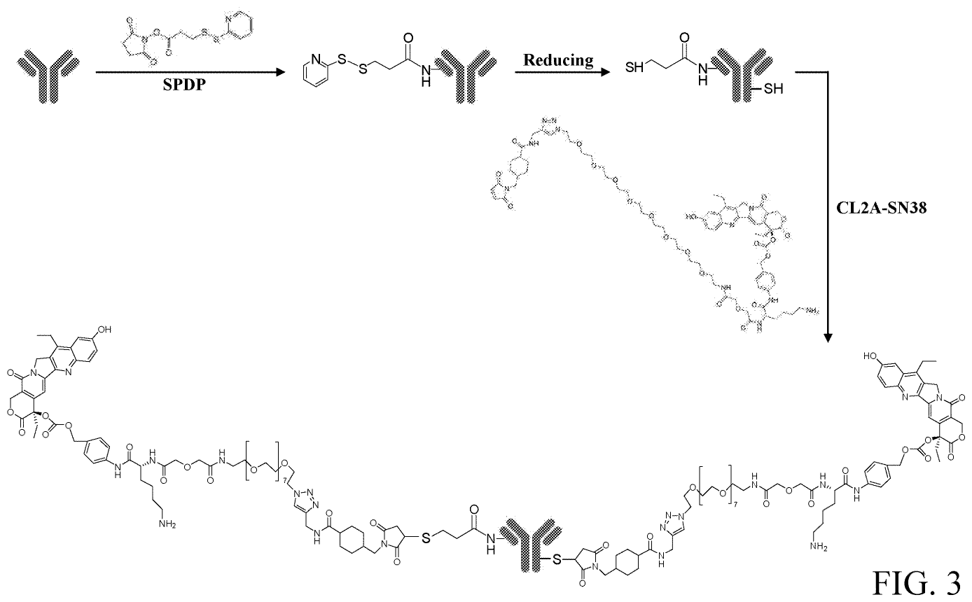

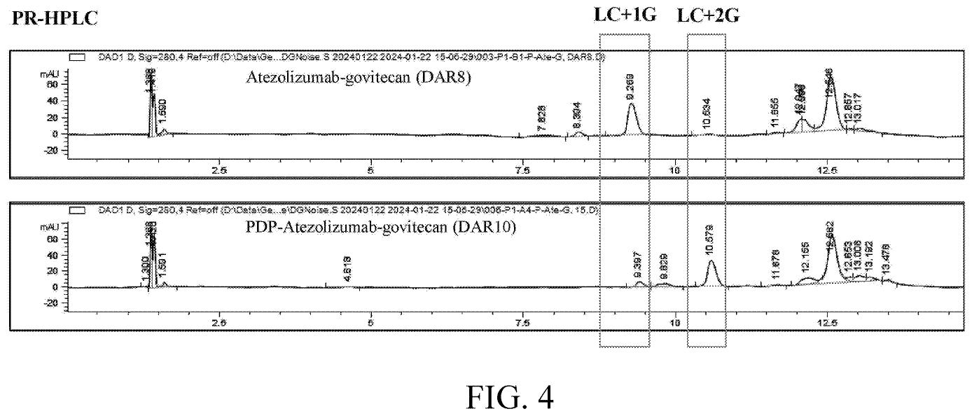

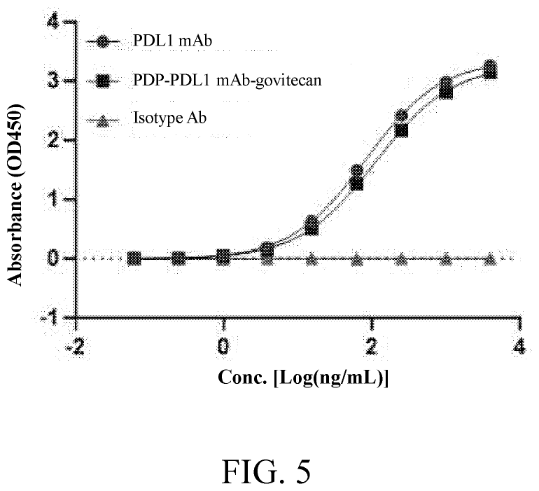

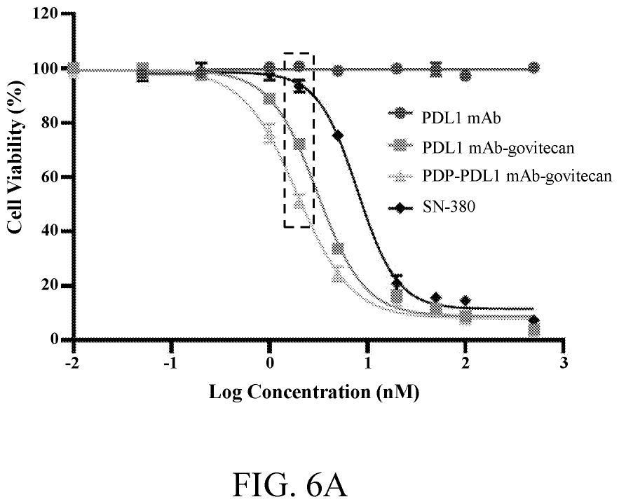

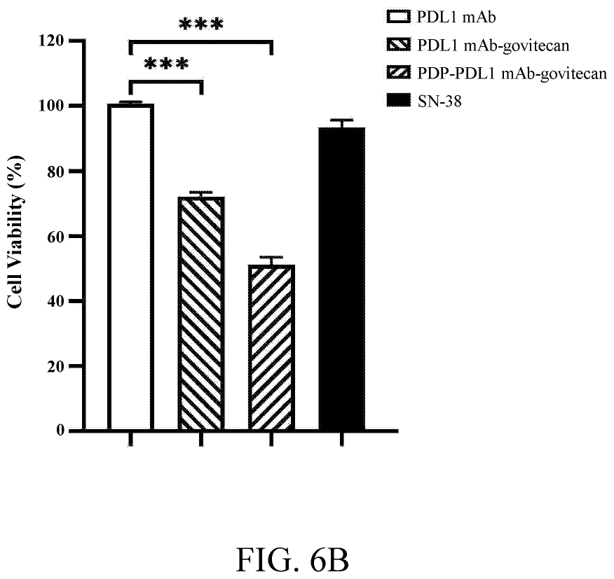

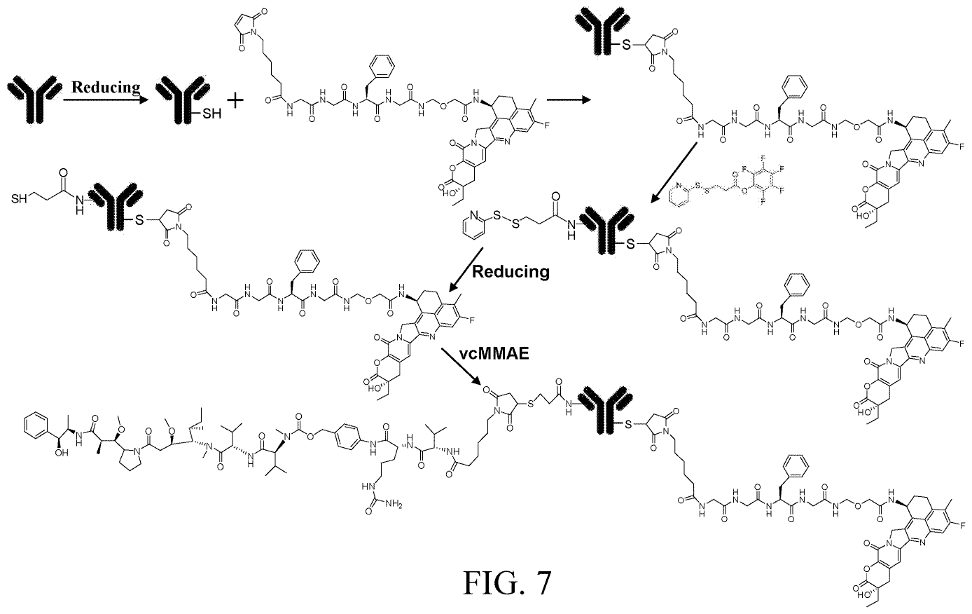

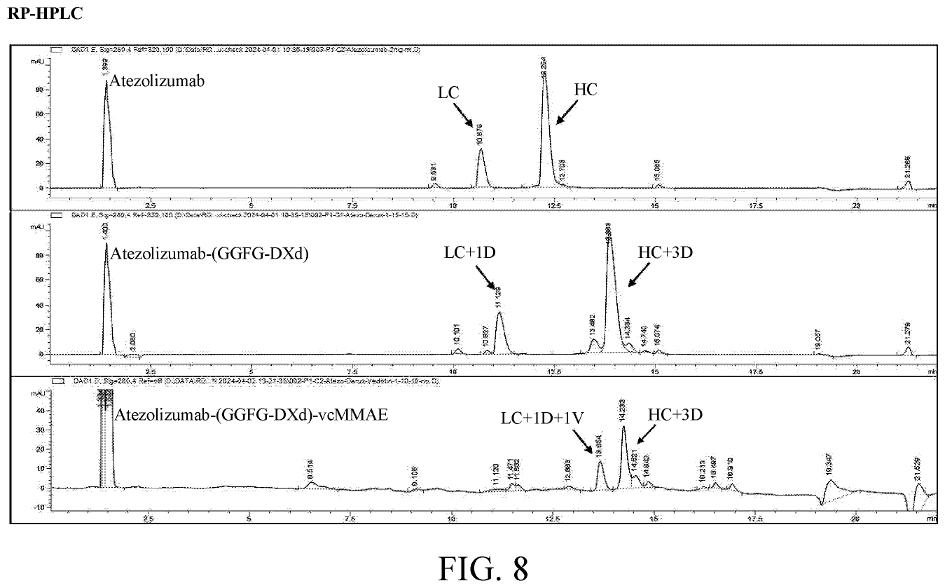

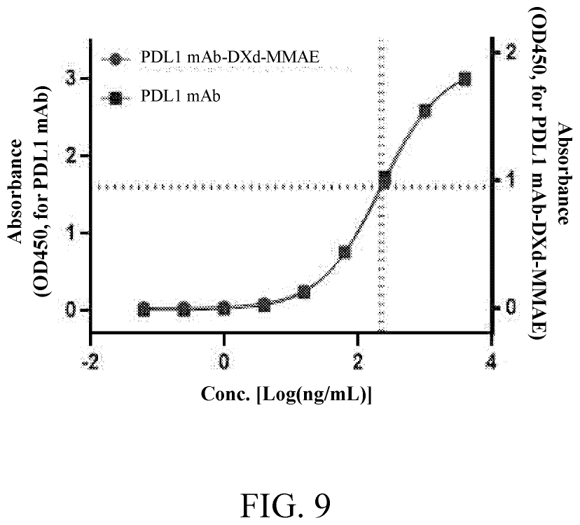

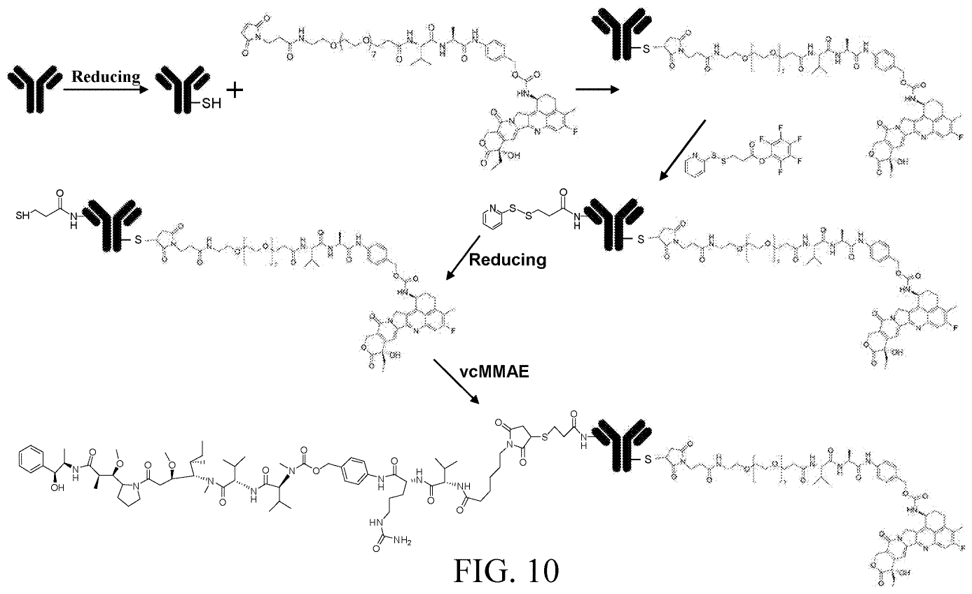

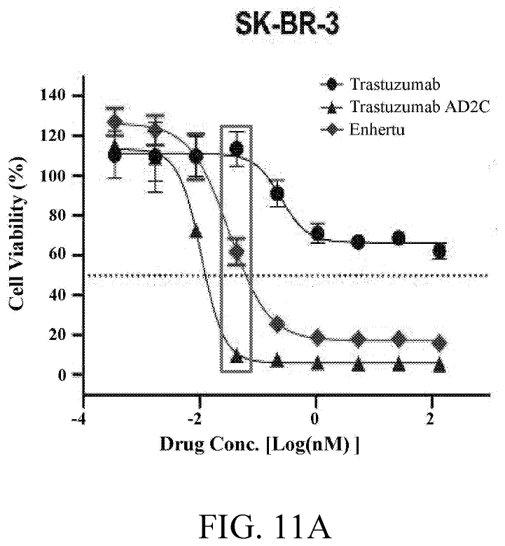

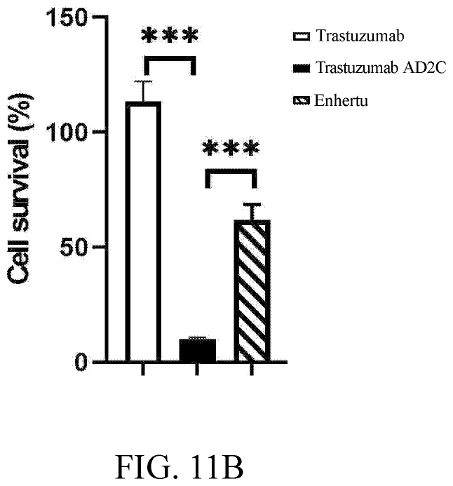

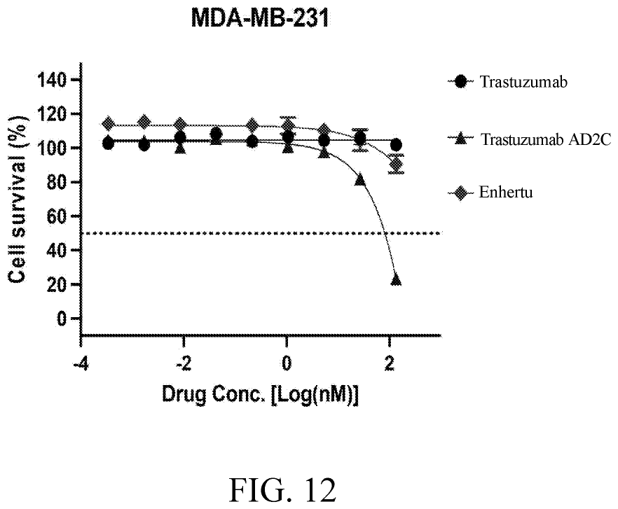

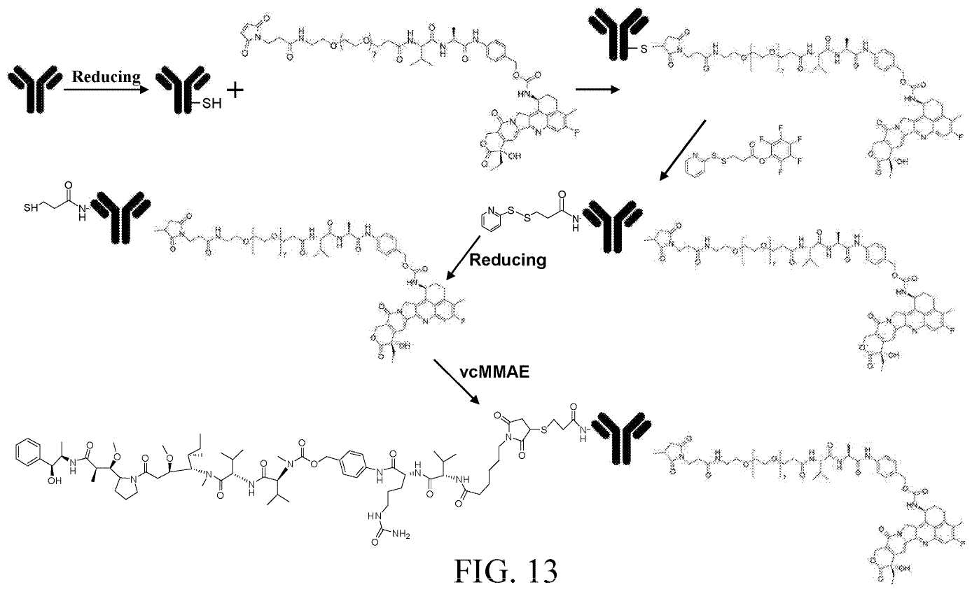

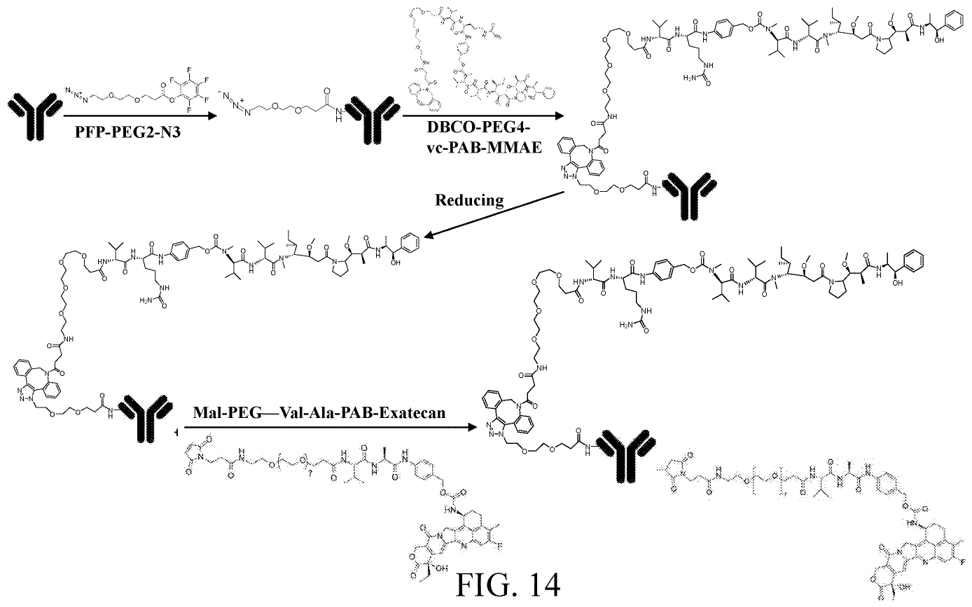

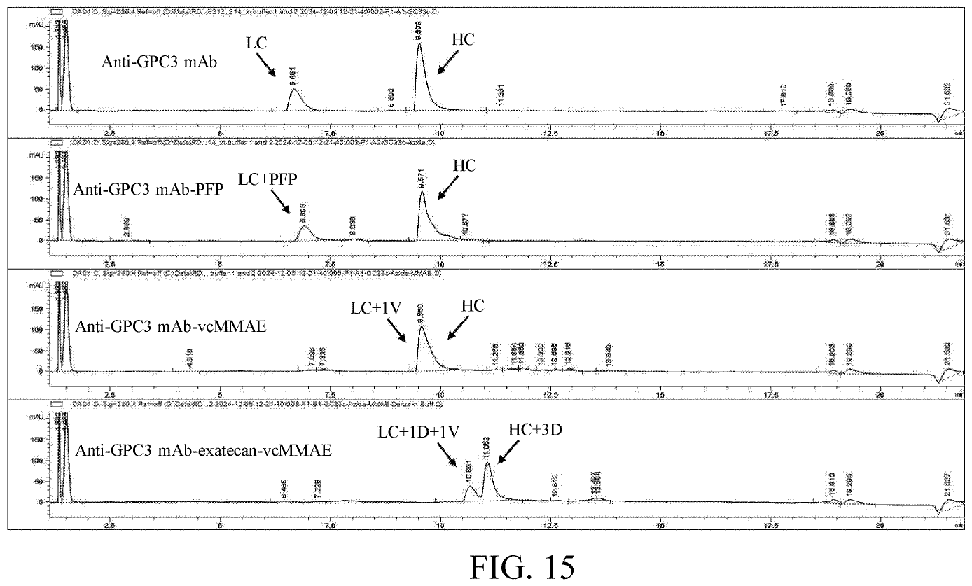

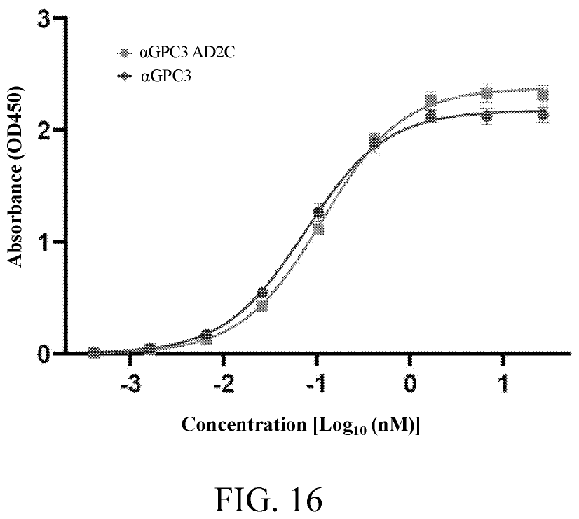

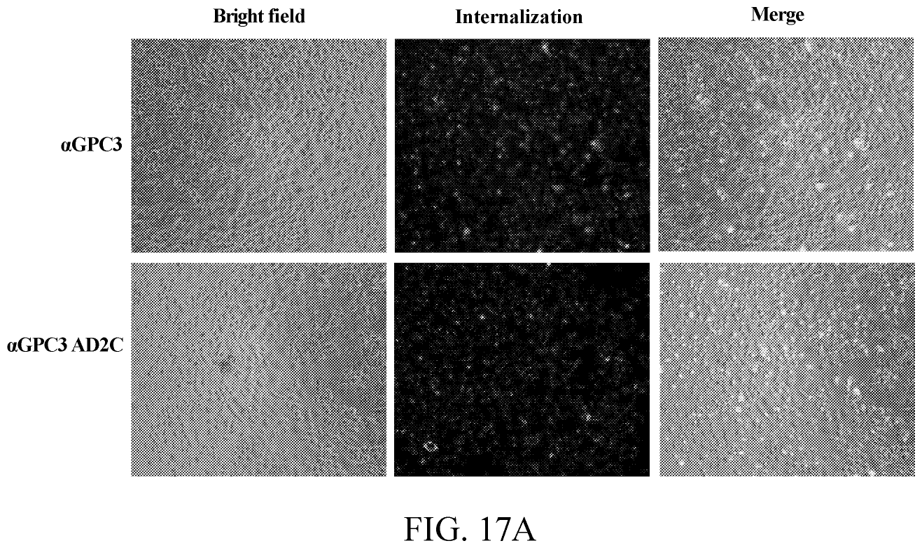

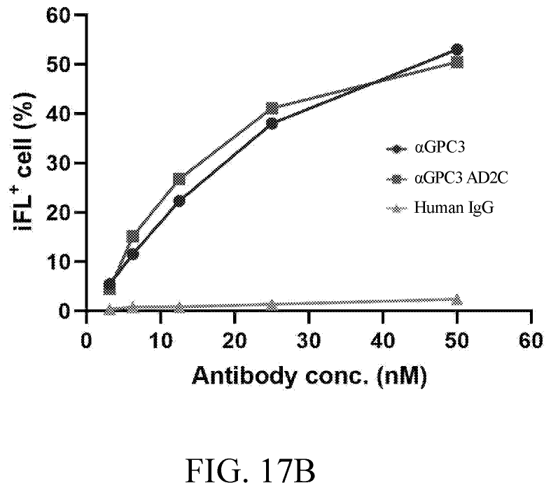

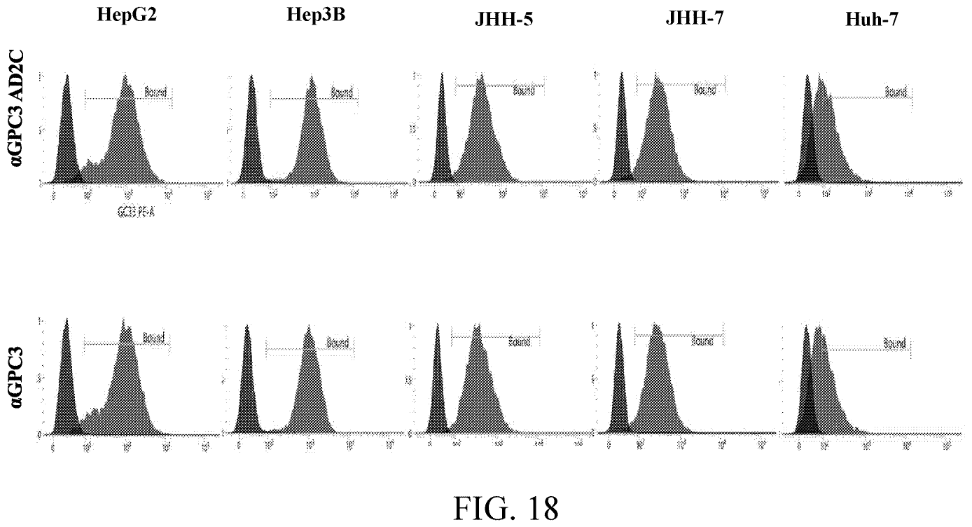

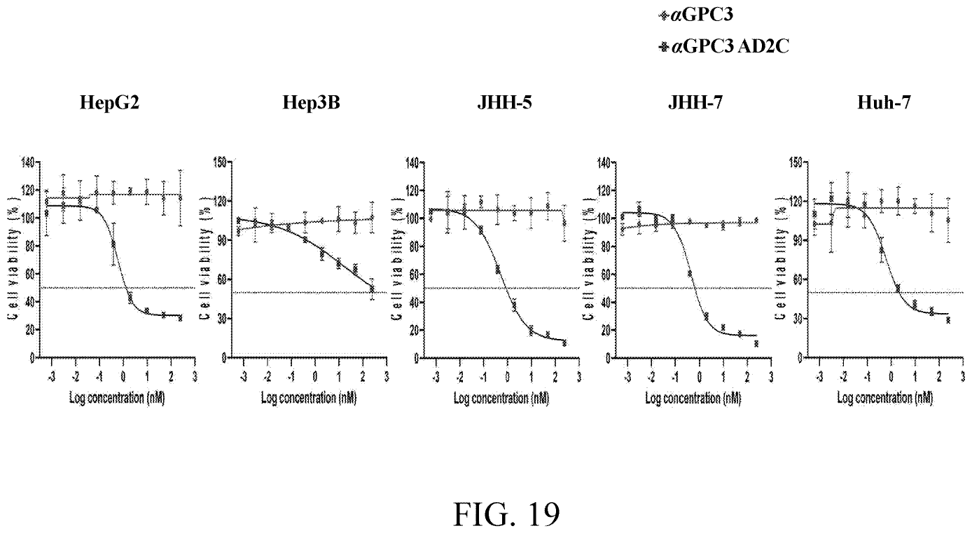

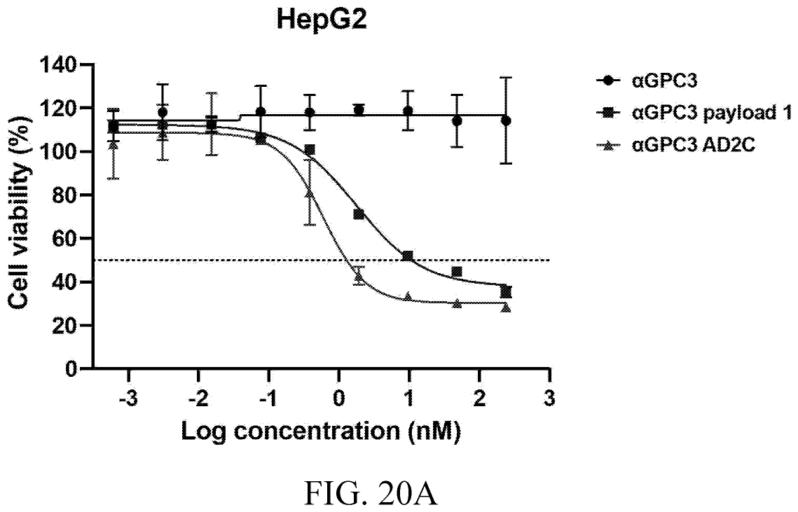

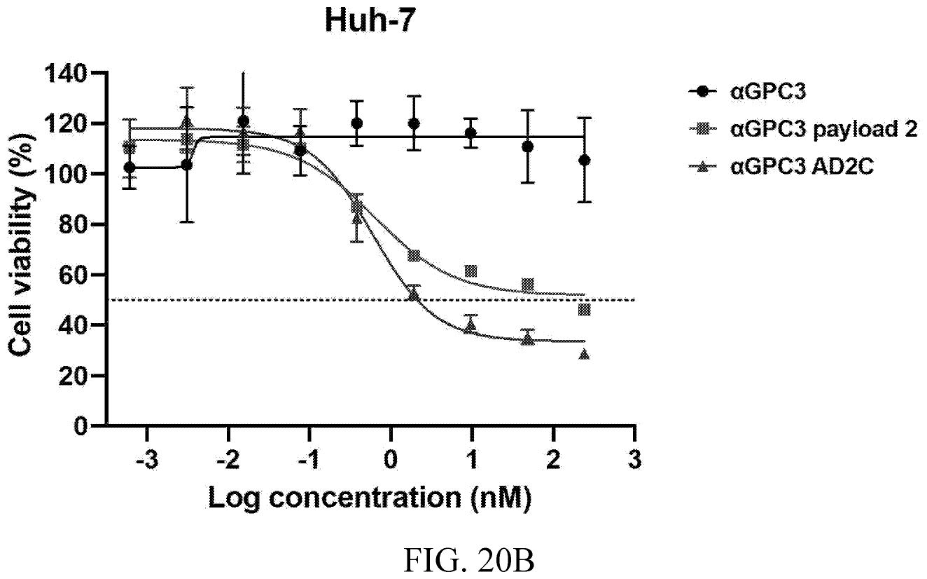

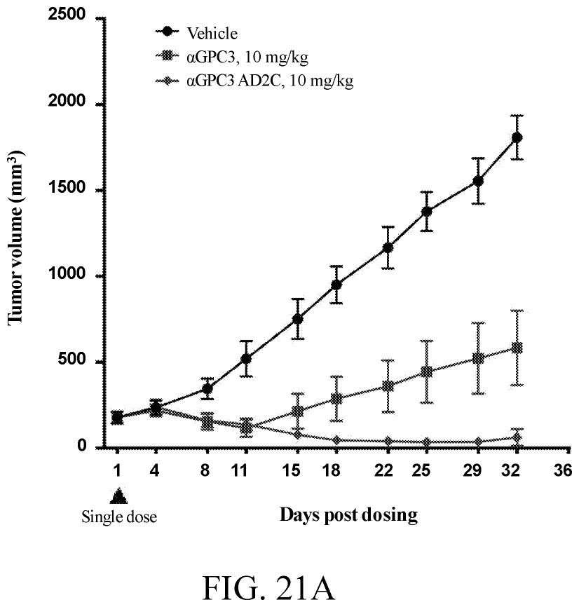

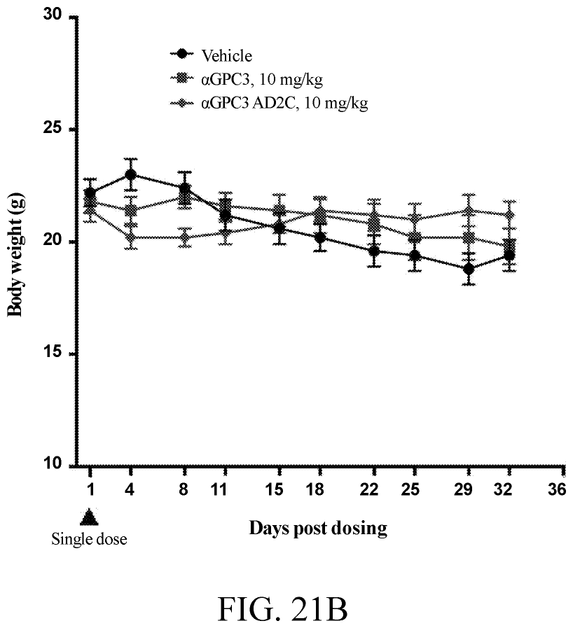

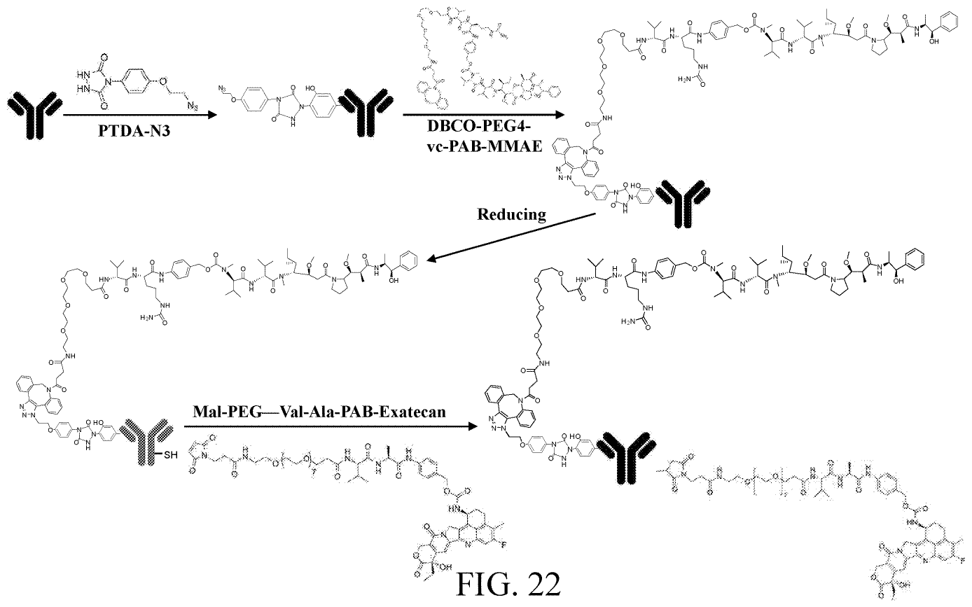

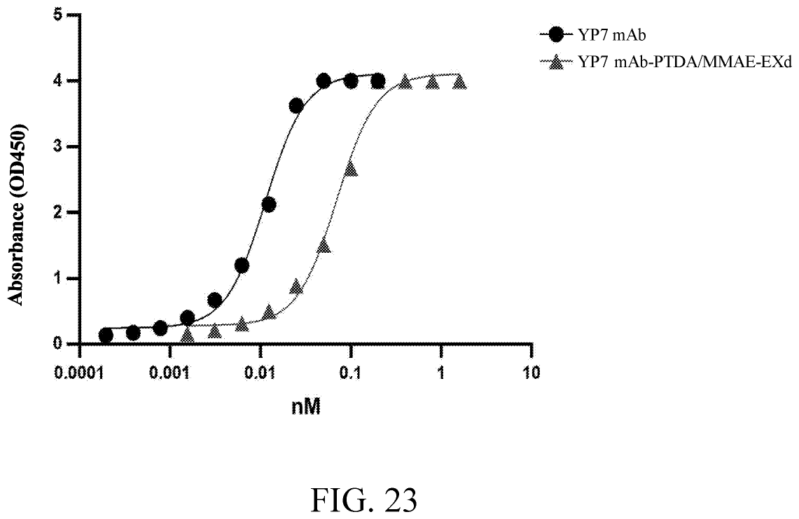

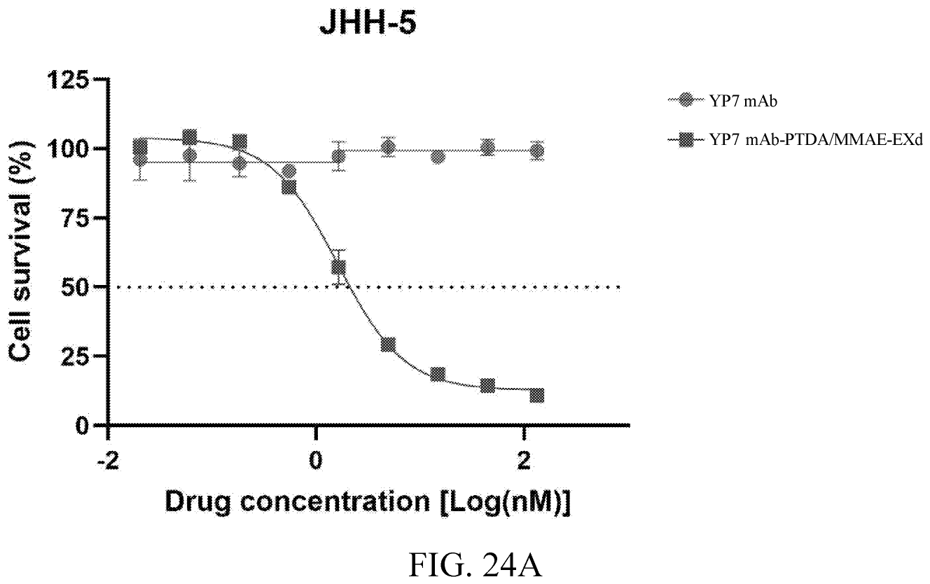

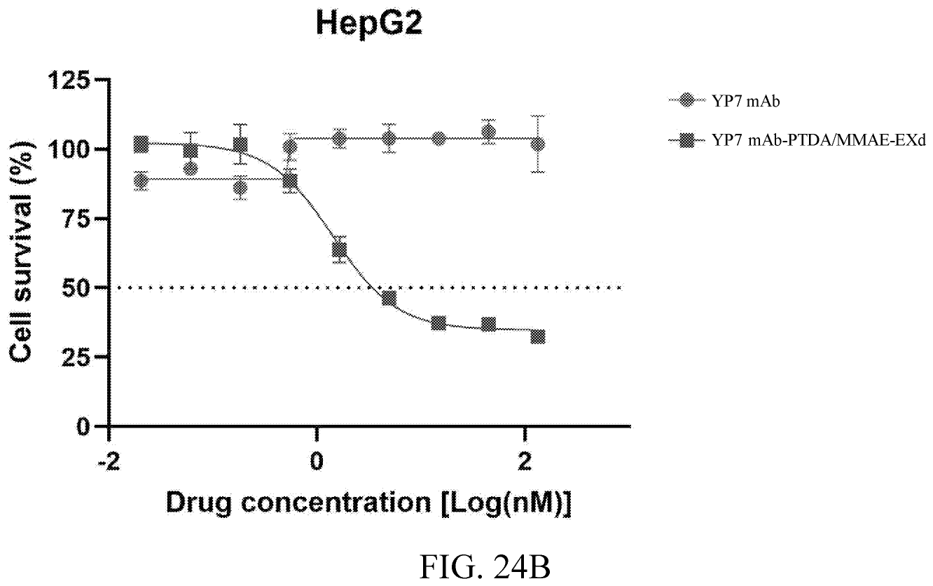

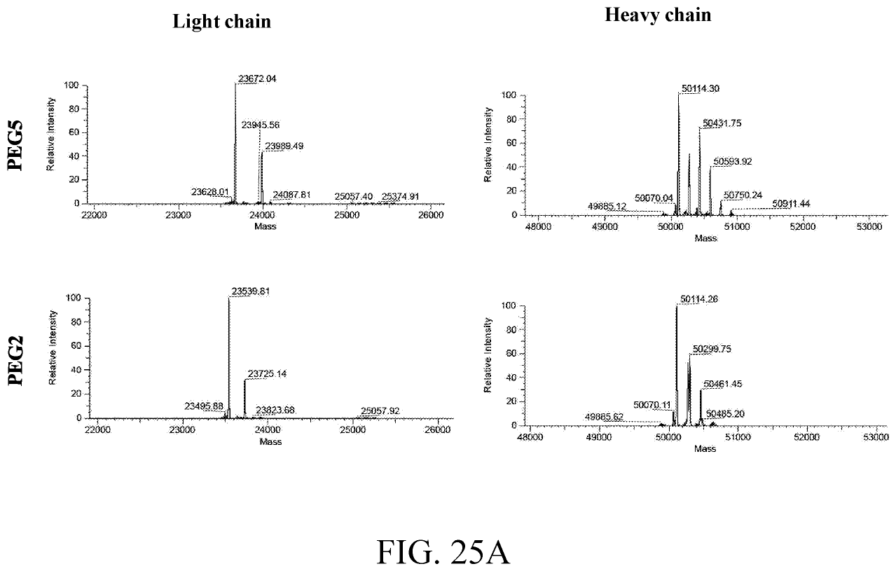

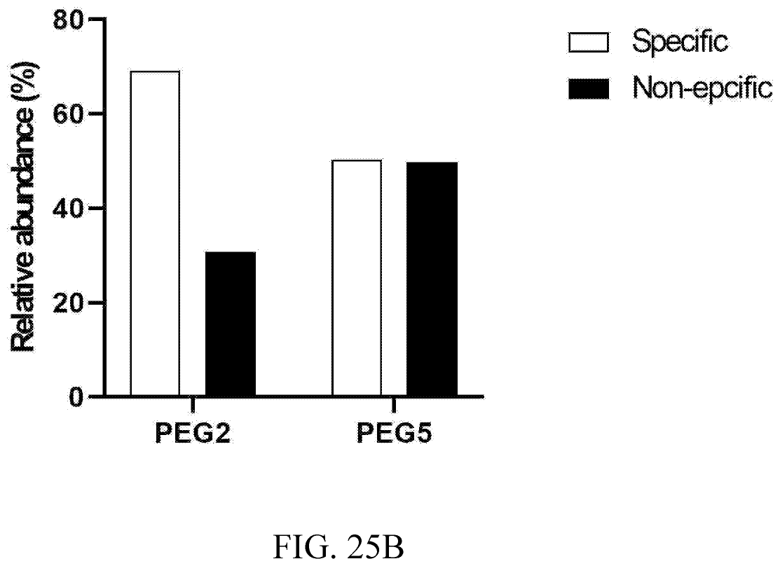

FIG. 4 provides Reverse phase HPLC (RP-HPLC) results of the product prepared according to the process described in FIG. 3.
Atezolizumab-govitecan (DAR8): the product prepared according to the procedure (A) conjugating 8 numbers of the first functional moieties (govitecan) described in FIG. 3.
PDP-Atezolizumab-govitecan (DAR10): the product prepared according to procedure (A) conjugating 8 numbers of the first functional moieties (govitecan) and procedure (B) conjugating 2 numbers of the second functional moiety (govitecan) described in FIG. 3.

FIG. 5 provides binding affinity results of the product prepared according to the process described in FIG. 3.
PDL1 mab: the antibody “Atezolizumab” (DAR0).
PDP-PDL1 mab-govitecan: the product prepared according to the procedure (A) conjugating 8 numbers of the first functional moieties (govitecan) and procedure (B) conjugating 2 numbers of the second functional moiety (govitecan) described in FIG. 3 (DAR10).
Isotype Ab: Isotype control antibody that lacks specificity to the target (PDL1) and therefore acts as negative controls.

FIG. 6A and FIG. 6B provide cytotoxicity results indicate that the product prepared according to the process described in FIG. 3 exhibits cytotoxicity against human triple-negative breast cancer cell line MDA-MB-231.
PDL1 mab: the antibody “Atezolizumab” (DAR0).
PDL1 mab-govitecan: the product prepared according to the procedure (A) conjugating 8 numbers of the first functional moieties (govitecan) described in FIG. 3 (DAR8).
PDP-PDL1 mab-govitecan: the product prepared according to the procedure (A) conjugating 8 numbers of the first functional moieties (govitecan) and procedure (B) conjugating 2 numbers of the second functional moiety (govitecan) described in FIG. 3 (DAR10).
SN-38: The payload of govitecan is used as positive control of cytotoxicity.

FIG. 7 illustrates an example process of obtaining a DAR10 ADC via procedure (A) conjugating 8 numbers of the first functional moieties (deruxtecan) and procedure (B) conjugating 2 numbers of the second functional moiety (MMAE) to an antibody (Atezolizumab), wherein the first functional moieties are linked to the cysteine residue on the surface of the light chain and on the surface of the heavy chain of the antibody via the first linker residue, and the second functional moieties are linked to the lysine residue on the surface of the light chain of the antibody via the second linker residue.

FIG. 8 provides Reverse phase HPLC (RP-HPLC) results of the product prepared according to the process described in FIG. 7.
Atezolizumab: the antibody (DAR0).
Atezolizumab-GGFG-DXd: the product prepared according to the procedure (A) conjugating 8 numbers of the first functional moieties described in FIG. 7 (DAR8).
Atezolizumab-(GGFG-DXd)-vcMMAE: the product prepared according to the procedure (A) conjugating 8 numbers of the first functional moieties and procedure (B) conjugating 2 numbers of the second functional moiety described in FIG. 7 (DAR10).

FIG. 9 provides binding affinity results of the product prepared according to the process described in FIG. 7.
PDL1 mab: the antibody “Atezolizumab” (DAR0).
PDL1 mab-DXd-MMAE: the product prepared according to the procedure (A) conjugating 8 numbers of the first functional moieties (DXd) and procedure (B) conjugating 2 numbers of the second functional moiety (MMAE) described in FIG. 7 (DAR10).

FIG. 10 illustrates an example process of obtaining a DAR10 ADC via procedure (A) conjugating 8 numbers of the first functional moieties (exatecan) and procedure (B) conjugating 2 numbers of the second functional moiety (MMAE) to an antibody (Trastuzumab), wherein the first functional moieties are linked to the cysteine residue on the surface of the light chain and on the surface of the heavy chain of the antibody via the first linker residue, and the second functional moieties are linked to the lysine residue on the surface of the light chain of the antibody via the second linker residue.

FIG. 11A and FIG. 11B provide cytotoxicity results indicating that the product prepared according to the process described in FIG. 10 exhibits cytotoxicity against human HER2-positive breast cancer cell line SK-BR-3.
Trastuzumab: the antibody “Trastuzumab” (DAR0).
Trastuzumab AD2C: the product prepared according to the procedure (A) conjugating 8 numbers of the first functional moieties (exatecan) and procedure (B) conjugating 2 numbers of the second functional moiety (MMAE) described in FIG. 10 (DAR10).
Enhertu: Trastuzumab-deruxtecan conjugation acts as positive control.

FIG. 12 provides cytotoxicity results indicating that the product prepared according to the process described in FIG. 10 exhibits cytotoxicity against human triple-negative breast cancer cell line MDA-MB-231.
Trastuzumab: the antibody “Trastuzumab” (DAR0).
Trastuzumab AD2C: the product prepared according to procedure (A) conjugating 8 numbers of the first functional moieties (exatecan) and procedure (B) conjugating 2 numbers of the second functional moiety (MMAE) described in FIG. 10 (DAR10).
Enhertu: Trastuzumab-deruxtecan conjugation acts as positive control.

FIG. 13 illustrates an example process of obtaining a DAR10 ADC via procedure (A) conjugating 8 numbers of the first functional moieties (exatecan) and procedure (B) conjugating 2 numbers of the second functional moiety (MMAE) to an antibody (anti-GPC3 monoclonal antibody), wherein the first functional moieties are linked to the cysteine residue on the surface of the light chain and on the surface of the heavy chain of the antibody via the first linker residue, and the second functional moieties are linked to the lysine residue on the surface of the light chain of the antibody via the second linker residue (the process using a PFP-based linker including a disulfide bond).

FIG. 14 illustrates an example process of obtaining a DAR10 ADC via procedure (A) conjugating 8 numbers of the first functional moieties (exatecan) and procedure (B) conjugating 2 numbers of the second functional moiety (MMAE) to an antibody (anti-GPC3 monoclonal antibody), wherein the first functional moieties are linked to the cysteine residue on the surface of the light chain and on the surface of the heavy chain of the antibody via the first linker residue, and the second functional moieties are linked to the lysine residue on the surface of the light chain of the antibody via the second linker residue (the process using a PFP-based linker including an azide functional group).

FIG. 15 provides Reverse phase HPLC (RP-HPLC) results of the product prepared according to the process described in FIG. 14.
Anti-GPC3 mAb: the antibody (DAR0).
Anti-GPC3 mAb-PFP: the antibody that reacted with PFP-based linker (DAR0).
Anti-GPC3 mAb-vcMMAE: the product prepared according to the procedure (B) conjugating 2 numbers of the second functional moieties described in FIG. 14 (DAR2).
Anti-GPC3 mAb-exatecan-vcMMAE: the product prepared according to the procedure (A) conjugating 8 numbers of the first functional moieties and procedure (B) conjugating 2 numbers of the second functional moiety described in FIG. 14 (DAR10).

FIG. 16 provides binding affinity results of the product prepared according to the process described in FIG. 14.
α GPC3: the anti-GPC3 monoclonal antibody (DAR0).
α GPC3 AD2C: the product prepared according to the procedure (A) conjugating 8 numbers of the first functional moieties (exatecan) and procedure (B) conjugating 2 numbers of the second functional moiety (MMAE) described in FIG. 14 (DAR10).

FIG. 17A and FIG. 17B provide antibody internalization results that indicate antibody internalization for human liver cancer cell line HepG2 is unchanged after the procedure (A) conjugating 8 numbers of the first functional moieties (exatecan) and procedure (B) conjugating 2 numbers of the second functional moiety (MMAE) to the antibody as described in FIG. 14.
α GPC3: the anti-GPC3 monoclonal antibody (DAR0).
α GPC3 AD2C: the product prepared according to the procedure (A) conjugating 8 numbers of the first functional moieties (exatecan) and procedure (B) conjugating 2 numbers of the second functional moiety (MMAE) described in FIG. 14 (DAR10).
Human IgG: IgG antibody that lacks specificity binding capacity to a cell-surface target protein GPC3 and therefore cannot trigger endocytosis to internalize the antibody into the cell, acts as negative control.

FIG. 18 provides results showing the binding capacity of the product prepared according to the process described in FIG. 14 to the cell-surface protein GPC3. The results indicate that the binding capacity of the anti-GPC3 monoclonal antibody to the cell-surface protein GPC3 on human liver cancer cell line HepG2, Hep3B, JHH-5, JHH-7, and Huh-7 is unchanged after the procedure (A) conjugating 8 numbers of the first functional moieties (exatecan) and procedure (B) conjugating 2 numbers of the second functional moiety (MMAE) to the antibody described in FIG. 14.
α GPC3: the anti-GPC3 monoclonal antibody (DAR0).
α GPC3 AD2C: the product prepared according to the procedure (A) conjugating 8 numbers of the first functional moieties (exatecan) and procedure (B) conjugating 2 numbers of the second functional moiety (MMAE) described in FIG. 14 (DAR10).

FIG. 19 provides cytotoxicity results indicate that the product prepared according to the process described in FIG. 14 exhibits cytotoxicity against human liver cancer cell line HepG2, Hep3B, JHH-5, JHH-7, and Huh-7.
α GPC3: the anti-GPC3 monoclonal antibody (DAR0).
α GPC3 AD2C: the product prepared according to the procedure (A) conjugating 8 numbers of the first functional moieties (exatecan) and procedure (B) conjugating 2 numbers of the second functional moiety (MMAE) described in FIG. 14 (DAR10).

FIG. 20A and FIG. 20B provide cytotoxicity results indicate that the product prepared according to the process described in FIG. 14 exhibits cytotoxicity against human liver cancer cell line HepG2 and Huh-7.
α GPC3: the anti-GPC3 monoclonal antibody (DAR0).
α GPC3-payload 1: the product prepared according to the procedure (A) conjugating 8 numbers of the first functional moieties (exatecan) described in FIG. 14 (DAR8).
α GPC3-payload 2: the product prepared according to the procedure (B) conjugating 2 numbers of the second functional moieties (MMAE) described in FIG. 14 (DAR2).
α GPC3 AD2C: the product prepared according to the procedure (A) conjugating 8 numbers of the first functional moieties (exatecan) and procedure (B) conjugating 2 numbers of the second functional moiety (MMAE) described in FIG. 14 (DAR10).

FIG. 21A and FIG. 21B provide results from mouse model studies demonstrating the anti-tumor activities of the product prepared according to embodiments of the present disclosure such as prepared according to the process described in FIG. 14. FIG. 21A provides the tumor volume in mice. FIG. 21B provides the body weight of the mice.

Vehicle group: mice were intravenously injected with normal saline.
α GPC3 group: mice were intravenously injected with the anti-GPC3 monoclonal antibody (DAR0).
α GPC3 AD2C group: mice were intravenously injected with the product prepared according to the procedure (A) conjugating 8 numbers of the first functional moieties (exatecan) and procedure (B) conjugating 2 numbers of the second functional moiety (MMAE) described in FIG. 14 (DAR10).

FIG. 22 illustrates an example process of obtaining a DAR10 ADC via procedure (A) conjugating 8 numbers of the first functional moieties ((4-NH2)exatecan) and procedure (A) conjugating 2 numbers of the second functional moiety (MMAE) to an antibody (anti-GPC3 monoclonal antibody), wherein the first functional moieties are linked to the cysteine residue on the surface of the light chain and on the surface of the heavy chain of the antibody via the first linker residue, and the second functional moieties are linked to the tyrosine residue on the surface of the light chain of the antibody via the second linker residue.

FIG. 23 provides binding affinity results of the product prepared according to the process described in FIG. 22.
YP7 mAb: the anti-GPC3 monoclonal antibody (DAR0).
YP7 mAb-PTDA/MMAE-EXd: the product prepared according to the procedure (A) conjugating 8 numbers of the first functional moieties (exatecan) and procedure (B) conjugating 2 numbers of the second functional moiety (MMAE) described in FIG. 22 (DAR10).

FIG. 24A and FIG. 24B provide cytotoxicity results indicate that the product prepared according to the process described in FIG. 22 exhibit cytotoxicity against human liver cancer cell line JHH-5 (FIG. 24A) and HepG2 (FIG. 24B).
YP7 mAb: the anti-GPC3 monoclonal antibody (DAR0).
YP7-PTDA/MMAE-Exd: the product prepared according to the procedure (A) conjugating 8 numbers of the first functional moieties (exatecan) and procedure (B) conjugating 2 numbers of the second functional moiety (MMAE) described in FIG. 22 (DAR10).

FIG. 25A and FIG. 25B provide Liquid chromatography-mass spectrometry (LC-MS) results of the antibody conjugated with different PFP-based linker (PEG2 group: using PFP-PEG2-N3 as PFP-based linker; PEG5 group: using PFP-PEG5-N3 as PFP-based linker). FIG. 25A provides the Light chain and Heavy chain Mass spectrometry results. FIG. 25B provides the percentage of specific conjugation of the PFP-based linker (the percentage of the PFP-based linker selectively reacting with a specific lysine residue in the light chain of the antibody).

### DETAILED DESCRIPTION OF THE INVENTION

### Definition

The term “PFP-based linker” as used herein refers to any compound or moiety that includes at least one pentafluorophenol (PFP) functional group and is structurally or functionally adapted to covalently connect or bridge two molecules (for example, adapted to covalently connect or bridge a functional (bio)molecule with a molecular tag), thereby forming a conjugate.

As used herein, the term “pH-sensitive structure” refers to a functional entity comprising at least one pH-labile bond or dissociable group, which remains stable at physiological pH (approximately 7.4) but undergoes hydrolysis, cleavage, protonation, or conformational change in an acidic environment (such as the tumor microenvironment with a pH of 6.5-6.8, or intracellular endosomes/lysosomes with a pH of 4.5-5.5). In some embodiments, the pH-sensitive structure may include, but is not limited to, hydrazone, oxime, phosphoramidate, acetal, ketal, vicinal dicarboxylic acid amide, silyl ether, maleamic acid, and orthoester.

As used herein, the term “hydrophobicity-masking entity” refers to a hydrophilic molecular unit covalently attached to a drug-linker or a conjugate structure. The entity possesses sufficient polarity or hydrophilicity to counteract, mask, or reduce the hydrophobic effects generated by the hydrophobic drug moiety associated therewith. Said entity is intended to increase the solubility of the overall complex in aqueous buffers, minimize intermolecular aggregation, reduce non-specific tissue binding, and optimize the in vivo pharmacokinetic properties thereof. In some embodiments, the hydrophobicity-masking entity may include, but is not limited to: (a) Polymers, such as polysarcosine (PSAR), polyethylene glycol (PEG), polyvinylpyrrolidone (PVP), or polyglycerol; (b) Peptide-based units, such as short chains composed of hydrophilic amino acids, comprising sequences of aspartic acid (Asp), glutamic acid (Glu), lysine (Lys), or arginine (Arg) (e.g., Glu-Val-Cit or Asp-Glu sequences); (c) Carbohydrates or polyols, such as glucuronide, mannose, or other monosaccharide/polysaccharide derivatives; and (d) Charged groups, such as highly polar functional groups including sulfonate, phosphate, or quaternary ammonium salts.

As used herein, the term “photoreactive moiety” refers to a chemical functional group that is activated upon exposure to or irradiation with light of a specific wavelength (e.g., ultraviolet (UV) light) to form covalent bonds with adjacent molecules. In some embodiments, the photoreactive moiety may include, but is not limited to, aryl azides, diazirines, benzophenones, and the like.

### Antibody Molecule

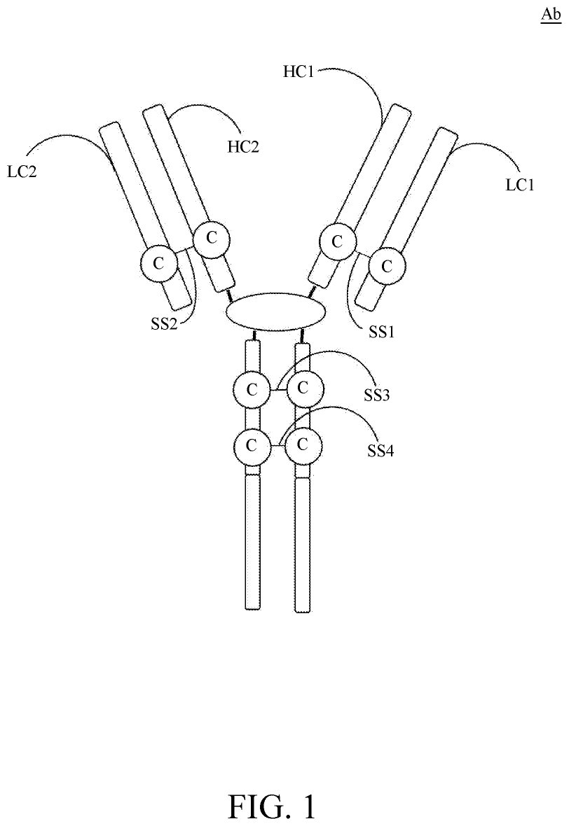

Please refer to FIG. 1, which schematically shows the structure of an antibody molecule (monomer). Antibody molecule (Ab) includes two heavy chains (HC1, HC2) and two light chains (LC1, LC2). There are 4 disulfide bonds (SS1, SS2, SS3, SS4) connecting these chains, so that each heavy chain is linked to a light chain and the two heavy chains are linked together, wherein each of the disulfide bond is formed between thiol groups in two cysteine residues. Therefore, in FIG. 1, there are 8 cysteine residues (C) in the surface of the antibody: 1 cysteine residue is in the surface of the first light chain (LC1), 1 cysteine residue is in the surface of the second light chain (LC2), 3 cysteine residues are in the surface of the first heavy chain (HC1), and 3 cysteine residues are in the surface of the second heavy chain (HC2). Moreover, the generic term “immunoglobulin (Ig)” is used for all antibodies and categorized into five different classes IgM, IgD, IgG, IgA, and IgE (refer to Section 1 “The structure of a typical antibody molecule” in Chapter 3 of the textbook “Immunobiology: The Immune System in Health and Disease”, 5th edition).

### Antibody Conjugate

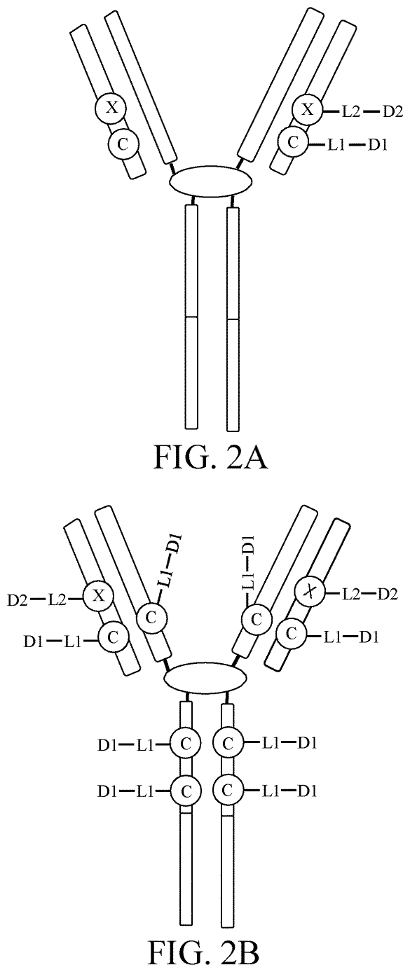

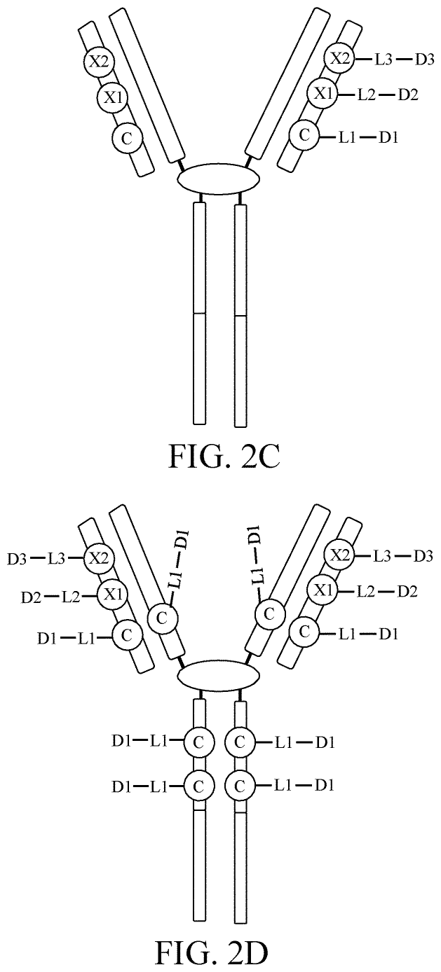

In some embodiments, an antibody conjugate has a light chain covalently linked to at least two different functional units. In some embodiments, the antibody conjugate has a light chain covalently linked to two different functional units. Please refer to FIG. 2A, the antibody conjugate includes an antibody having two light chains and two heavy chains, wherein each of the light chains includes on its surface a first amino acid residue (C) and a second amino acid residue (X); a first functional moiety (D1) is conjugated to the first amino acid residue (C) on the surface of a light chain via the first linker residue (L1), and a second functional moiety (D2) is conjugated to the second amino acid residue (X) on the surface of the same light chain via the second linker residue (L2). In some embodiments, the first amino acid residue (C) is a cysteine residue. In some embodiments, the second amino acid residue (X) is a lysine residue or a tyrosine residue.

In some embodiments, the antibody conjugate has 8 numbers of the first functional unit and 2 numbers of the second functional unit. Please refer to FIG. 2B, the antibody conjugate includes an antibody having 2 light chains and 2 heavy chains, wherein each of the light chains includes on its surface a first amino acid residue (C) and a second amino acid residue (X), and each of the heavy chains includes on its surface three numbers of third amino acid residue (C); first functional moieties (D1) are conjugated to the first amino acid residues (C) on the surface of a light chains or the third amino acid residues (C) on the surface of a heavy chains via the first linker residues (L1), and second functional moieties (D2) are conjugated to the second amino acid residues (X) on the surface of the light chains via the second linker residues (L2), so that each one of the light chains is covalently linked to two different functional units (L1-D1 and L2-D2), and each one of the heavy chains is covalently linked to 3 functional units (L1-D1). Therefore, there are 10 functional units conjugated to an antibody (if D1 and D2 are drugs, the drug antibody ratio (DAR value) is 10; refer to DAR10 ADCs). In some embodiments, the first amino acid residue (C) is a cysteine residue. In some embodiments, the second amino acid residue (X) is a lysine residue or a tyrosine residue. In some embodiments, the third amino acid residue (C) is a cysteine residue.

In some embodiments, the antibody conjugate has a light chain covalently linked to three different functional units. Please refer to FIG. 2C, the antibody conjugate includes an antibody having 2 light chains and 2 heavy chains, wherein each of the light chains includes on its surface a first amino acid residue (C), a second amino acid residue (X1), and a fourth amino acid residue (X2); a first functional moiety (D1) is conjugated to the first amino acid residue (C) on the surface of a light chain via the first linker residue (L1), a second functional moiety (D2) is conjugated to the second amino acid residue (X1) on the surface of the same light chain via the second linker residue (L2), and a third functional moiety (D3) is conjugated to the fourth amino acid residue (X2) on the surface of the same light chain via the third linker residue (L3). In some embodiments, the first amino acid residue (C) is a cysteine residue. In some embodiments, the second amino acid residue (X1) is a lysine residue. In some embodiments, the fourth amino acid residue (X2) is a tyrosine residue.

In some embodiments, an antibody conjugate has 8 numbers of the first functional unit, 2 numbers of the second functional unit, and 2 numbers of the third functional unit. Please refer to FIG. 2D, the antibody conjugate includes an antibody having 2 light chains and 2 heavy chains, wherein each of the light chains includes on its surface a first amino acid residue (C), a second amino acid residue (X1), and a fourth amino acid residue (X2); and each of the heavy chains includes on its surface three numbers of third amino acid residue (C); first functional moieties (D1) are conjugated to the first amino acid residues (C) on the surface of a light chains or the third amino acid residues (C) on the surface of a heavy chains via the first linker residues (L1), second functional moieties (D2) are conjugated to the second amino acid residues (X1) on the surface of the light chains via the second linker residues (L2), and third functional moieties (D3) are conjugated to the fourth amino acid residues (X2) on the surface of the light chains via the third linker residues (L3), so that each one of the light chains is covalently linked to three different functional units (L1-D1, L2-D2, and L3-D3), and each one of the heavy chains is covalently linked to 3 functional units (L1-D1). Therefore, there are 12 functional units conjugated to an antibody (if D1, D2, and D3 are drugs, the drug antibody ratio (DAR value) is 12; refer to DAR12 ADCs).

In some embodiments, the molecular weight of each one of first functional unit (L1-D1), the second functional unit (L2-D2), and the third functional unit (L3-D3) is 0.5 to 20 kDa.

In some embodiments, the first linker residue includes a structure represented by the following formula:

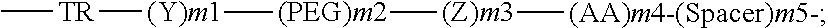

wherein,
TR is a residue including a sulfhydryl-reactive group conjugated to one of the first and the at least one third amino acid residues;
Y is a Y molecule residue, —(CH₂)m6-(C═O)—, or —(CH₂)m7-(C═O)—NH—(CH₂)m8-, wherein the Y molecule residue includes an alkyl group, a cycloalkyl group, a branched alkyl group, a glucuronide, a disulfide, an ester, a hydrazone, a thioether, or any combination thereof;
m1 is 0 or 1, m6 is an integer from 0 to 10, m7 is an integer from 0 to 10, m8 is an integer from 0 to 10;
-(PEG)m2- represents —(CH₂—CH₂—O)m2-, and m2 is an integer from 0 to 30;
Z is an Z molecule residue including an alkyl group, a pH-sensitive structure, a hydrazone, a disulfide, a glucuronide, a pyrophosphate, —(C═O)—, or other structure, and m3 is an integer from 0 to 10;
-(AA)m4- represents m4 amino acid residues selected from the group consisting of the basic amino acid, the neutral amino acid, the hydrophobic amino acid, the polar acidic amino acid, or any combination thereof, wherein m4 is an integer from 0 to 30;
Spacer is a self-immolative molecule connecting an oxygen or nitrogen of the second functional moiety, wherein the spacer is hemiaminal-related molecule, heterocyclic molecule, Mannich-type molecule, PABC, an aforementioned molecule substituted with a hydrophobicity-masking entity, or other spacer, and m5 is 0 or 1, and
wherein the antibody or antigen-binding fragment thereof is connected to the terminal of TR.

In some embodiments, m3 may be 0 or 1.

In some embodiments, the second linker residue and/or the third linker residue includes a structure represented by the following formula:

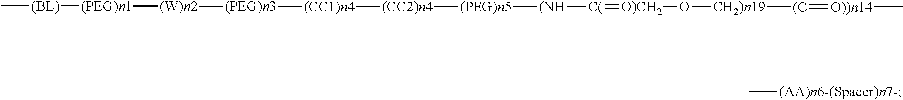

wherein,
-(PEG)n1- represents —(CH₂—CH₂—O)n1-, and n1 is an integer from 0 to 30;
W is a functional linker residue including —C(═O)—(CH₂)n8-S-(Succinimid-3-yl-N)—(CH₂)n16-, —C(═O)—(CH₂)n10-, maleimidocaproic acid, an alkyne, a 3-(pyridin-2-yldisulfanyl) propanoate (PDP) residue, an azide, an alkene, an ester, a sulfhydryl group, a hydroxyl group, a thiol, an aldehyde, a ketone, a photoreactive moiety, a glucuronide, a glucurunoside, —S—, or —S—R³—, and n2 is 0 or 1, wherein n8 is an integer from 0 to 10, n10 is an integer from 0 to 10, and n16 is an integer from 0 to 10;
-(Succinimid-3-yl-N)— has a structure represented by the following formula:

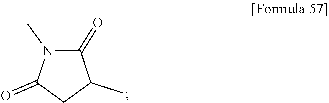

R³ is selected from the group consisting of C₁₋₆ alkyl, C₁₋₆ alkoxy, halo, halo(C₁₋₆)alkyl, hydroxy, C₁₋₆ alkylthio, C₁₋₆ alkoxycarbonyl, cyano, nitro, amino, mono- or di-C₁₋₆ alkylamino, C₁₋₆ acylamino, C₁₋₆ acyl, carboxy, C₁₋₆ alkoxycarbonyl, C₁₋₆ alkylsulfonyl, C₁₋₆ alkylsulfonamido, and phenyl or substituted phenyl;
-(PEG)n3- represents —(CH₂—CH₂—O)n3-, and n3 is an integer from 0 to 30;
CC1 is a reaction moiety of a bioorthogonal pair;
CC2 is a reaction moiety of the bioorthogonal pair; and n4 is 0 or 1;
-(PEG)n5- represents —(CH₂—CH₂—O)n5-, and n5 is an integer from 0 to 30;
-(AA)n6- represents n6 amino acid residues selected from the group consisting of the basic amino acid, the neutral amino acid, the hydrophobic amino acid, the polar acidic amino acid, and any combination thereof, wherein n6 is an integer from 0 to 30;
Spacer is a self-immolative molecule connecting an oxygen or nitrogen of the second functional moiety, wherein the spacer is hemiaminal-related molecule, heterocyclic molecule, Mannich-type molecule, PABC, an aforementioned molecule substituted with a hydrophobicity-masking entity, or other spacer, and n7 is 0 or 1;
n14 is 0 or 1, and n19 is 0 or 1; and
BL is a single bond or a functional linker residue including amino acid reactive moiety, and wherein the antibody or antigen-binding fragment thereof is connected to the terminal of BL.

In some embodiments, the n6 amino acid residues are linked in sequence.

In some embodiments, the m4 amino acid residues are linked in sequence.

In some embodiments, -(BL)-(PEG)n1-(W)n2-(PEG)n3-(CC1)n4- has a structure represented by the following formula:

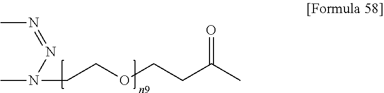

and wherein the formula:

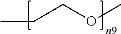

includes 0, 1, or 2 number of —(CH₂—CH₂—O)—.

### Pharmaceutical Compositions

Provided herein are also pharmaceutical compositions including the antibody conjugates described herein and a pharmaceutically acceptable carrier. In some embodiments, the pharmaceutical composition can further comprise one or more additional active agents, such as an active agent suitable for treating the diseases that the pharmaceutical compositions are intended for.

The term “pharmaceutically acceptable carrier” or “pharmaceutically acceptable excipient” refers to a material that is suitable for drug administration to an individual along with an active agent without causing undesirable biological effects or interacting in a deleterious manner with any of the other components of the pharmaceutical composition.

In some embodiments, the pharmaceutical composition is an aqueous formulation. Such a formulation is typically a solution or a suspension, but can also include colloids, dispersions, emulsions, and multi-phase materials. The term “aqueous formulation” is defined as a formulation including at least 50% w/w water. Likewise, the term “aqueous solution” is defined as a solution including at least 50% w/w water, and the term “aqueous suspension” is defined as a suspension including at least 50% w/w water. Pharmaceutically acceptable carriers that can be used in pharmaceutical composition include any and all solvents, dispersion media, coatings, antibacterial and antifungal agents, isotonic and absorption delaying agents, and the like that are physiologically compatible. Pharmaceutically acceptable carriers can include, for example, buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminium hydroxide); and preservatives. In some embodiments, the pharmaceutical compositions are cryopreserved, to which the physician or the patient adds solvents and/or diluents prior to use; and cryopreservation solutions which can be used in the pharmaceutical compositions described herein include, for example, DMSO.

In some embodiments, the pharmaceutical composition are substantially free of contaminant. In some embodiments, the pharmaceutical composition have no detectable levels of contaminants. The contaminants include, for example, endotoxin, mycoplasma, bacterial components, and feeder cells (e.g., transformed cells).

Pharmaceutical composition can be formulated, for example, for parenteral (e.g., intravenous, subcutaneous, intraperitoneal, intramuscular, intrathecal) administration. In some embodiments, the pharmaceutical composition are formulated for parenteral administration. In some embodiments, the carriers included in the pharmaceutical composition are suitable for parenteral administration (e.g., by injection or infusion). In some embodiments, the pharmaceutical composition are formulated for intravenous administration. In some embodiments, the carriers included in the pharmaceutical composition are suitable for intravenous administration.

### Methods of Uses

The antibody conjugations and pharmaceutical composition can be used as a medicament. In some embodiments, provided herein are methods for treating a disease or disorder in a subject in need thereof including administering the antibody conjugations or pharmaceutical compositions described herein to the subject. In some embodiments, provided herein are uses of the antibody conjugations or pharmaceutical compositions described herein for treating a disease or disorder in a subject in need thereof. In some embodiments, provided herein are uses of the antibody conjugations or pharmaceutical compositions described herein for the preparation of a medicament for the treatment of a disease or disorder in a subject in need thereof.

The term “treat” and its grammatical equivalents as used herein in connection with a disease or a condition, or a subject having a disease or a condition refer to an action that suppresses, eliminates, reduces, and/or ameliorates a symptom, the severity of the symptom, and/or the frequency of the symptom associated with the disease or disorder being treated. For example, when used in reference to a cancer or tumor, the term “treat” and its grammatical equivalents refer to an action that reduces the severity of the cancer or tumor, or retards or slows the progression of the cancer or tumor, including (a) inhibiting the growth, or arresting development of the cancer or tumor, (b) causing regression of the cancer or tumor, or (c) delaying, ameliorating or minimizing one or more symptoms associated with the presence of the cancer or tumor.

The term “administer” and its grammatical equivalents as used herein refer to the act of delivering, or causing to be delivered, a therapeutic or a pharmaceutical composition to the body of a subject by a method described herein or otherwise known in the art. The therapeutic can be a compound, a polypeptide, an antibody, a cell, or a population of cells. Administering a therapeutic or a pharmaceutical composition includes prescribing a therapeutic or a pharmaceutical composition to be delivered into the body of a subject.

The terms “effective amount,” “therapeutically effective amount,” and their grammatical equivalents as used herein refer to the administration of an agent to a subject, either alone or as a part of a pharmaceutical composition and either in a single dose or as part of a series of doses, in an amount that is capable of having any detectable, positive effect on any symptom, aspect, or characteristics of a disease, disorder or condition when administered to the subject. The therapeutically effective amount can be ascertained by measuring relevant physiological effects. The exact amount required vary from subject to subject, depending on the age, weight, and general condition of the subject, the severity of the condition being treated, the judgment of the clinician, and the like. An appropriate “effective amount” in any individual case can be determined by one of ordinary skill in the art using routine experimentation.

The term “subject” as used herein refers to any animal (e.g., a vertebrate). The subjects include, but are not limited to, humans, non-human primates, simians, canines, felines, rodents, and the like, which is to be the recipient of a particular treatment. A subject can be a human. A subject can be a mammal. A subject can be a farm animal. As subject can be a pet. A subject can have a particular disease or condition.

In some embodiments, the antibody conjugations and pharmaceutical composition can be used in the treatment of cancer, an infectious disease or an inflammatory disease. In some embodiments, the antibody conjugations and pharmaceutical composition can be used in modulating an immune response in a subject in need thereof. In some embodiments, provided herein are methods of treating a cancer, an infectious disease or an inflammatory disease in a subject in need thereof, including administering a therapeutically effective amount of the antibody conjugations described herein. Alternatively, a therapeutically effective amount of the pharmaceutical composition including the antibody conjugations is administered.

In some embodiments, the disease or disorder can be cancer, tumor, autoimmune disease, neuronal disease, HIV infection, hematopoietic cell-related diseases, metabolic syndrome, pathogenic disease, viral infection, fungal infection, protozoan infection, or bacterial infection. As such, the cell population, including those prepared by methods described herein, as well as the pharmaceutical composition, can be used in, for example, cancer treatment, autoimmune disease treatment, neuronal disease treatment, human immunodeficiency virus (HIV) eradication, hematopoietic cell-related diseases, metabolic syndrome treatment, pathogenic disease treatment, treatment of viral infection, fungal infection, protozoan infection, and treatment of bacterial infection. In some embodiments, the cell populations and pharmaceutical compositions described herein can be used to treat a disease or disorder associated with abnormal cells. In some embodiments, the disease or disorder is a hyperproliferative disease.

In some embodiments, the uses of the antibody conjugates or pharmaceutical composition in the treatment of a tumor or cancer. In some embodiments, provided herein are methods of treating a tumor or cancer in a subject in need thereof, including administering the antibody conjugates or pharmaceutical composition to the subject. In some embodiments, the tumor or cancer is a solid tumor. In some embodiments, the tumor or cancer is a hematological cancer, or liquid cancer.

In some embodiments, the disease or disorder that can be treated with the antibody conjugates or pharmaceutical composition is acanthoma, acinic cell carcinoma, acoustic neuroma, acral lentiginous melanoma, acrospiroma, acute eosinophilic leukemia, acute lymphoblastic leukemia, acute megakaryoblastic leukemia, acute monocytic leukemia, acute myeloblastic leukemia with maturation, acute myeloid dendritic cell leukemia, acute myeloid leukemia, acute promyelocytic leukemia, adamantinoma, adenocarcinoma, adenoid cystic carcinoma, adenoma, adenomatoid odontogenic tumor, adrenocortical carcinoma, adult t-cell leukemia, aggressive NK-cell leukemia, AIDS-related cancers, AIDS-related lymphoma, alveolar soft part sarcoma, ameloblastic fibroma, anal cancer, anaplastic large cell lymphoma, anaplastic thyroid cancer, angioimmunoblastic t-cell lymphoma, angiomyolipoma, angiosarcoma, appendix cancer, astrocytoma, atypical teratoid rhabdoid tumor, basal cell carcinoma, basal-like carcinoma, b-cell leukemia, b-cell lymphoma, bellini duct carcinoma, biliary tract cancer, bladder cancer, blastoma, bone cancer, bone tumor, brain stem glioma, brain tumor, breast cancer, brenner tumor, bronchial tumor, bronchioloalveolar carcinoma, brown tumor, burkitt's lymphoma, cancer of unknown primary site, carcinoid tumor, carcinoma, carcinoma in situ, carcinoma of the penis, carcinoma of unknown primary site, carcinosarcoma, castleman's disease, central nervous system embryonal tumor, cerebellar astrocytoma, cerebral astrocytoma, cervical cancer, cholangiocarcinoma, chondroma, chondrosarcoma, chordoma, choriocarcinoma, choroid plexus papilloma, chronic lymphocytic leukemia, chronic monocytic leukemia, chronic myelogenous leukemia, chronic myeloproliferative disorder, chronic neutrophilic leukemia, clear-cell tumor, colon cancer, colorectal cancer, craniopharyngioma, cutaneous T-cell lymphoma, Degos disease, dermatofibrosarcoma protuberans, dermoid cyst, desmoplastic small round cell tumor, diffuse large B cell lymphoma, dysembryoplastic neuroepithelial tumor, embryonal carcinoma, endodermal sinus tumor, endometrial cancer, endometrial uterine cancer, endometrioid tumor, enteropathy-associated T-cell lymphoma, ependymoblastoma, ependymoma, epithelioid sarcoma, erythroleukemia, esophageal cancer, esthesioneuroblastoma, Ewing family of tumor, Ewing family sarcoma, Ewing's sarcoma, extracranial germ cell tumor, extragonadal germ cell tumor, extrahepatic bile duct cancer, extramammary Paget's disease, fallopian tube cancer, fetus in fetus, fibroma, fibrosarcoma, follicular lymphoma, follicular thyroid cancer, gallbladder cancer, gallbladder cancer, ganglioglioma, ganglioneuroma, gastric cancer, gastric lymphoma, gastrointestinal cancer, gastrointestinal carcinoid tumor, gastrointestinal stromal tumor, gastrointestinal stromal tumor, germ cell tumor, germinoma, gestational choriocarcinoma, gestational trophoblastic tumor, giant cell tumor of bone, glioblastoma multiforme, glioma, gliomatosis cerebri, glomus tumor, glucagonoma, gonadoblastoma, granulosa cell tumor, hairy cell leukemia, hairy cell leukemia, head and neck cancer, heart cancer, hemangioblastoma, hemangiopericytoma, hemangiosarcoma, hematological malignancy, hepatocellular carcinoma, hepatosplenic T-cell lymphoma, hereditary breast-ovarian cancer syndrome, Hodgkin lymphoma, Hodgkin's lymphoma, hypopharyngeal cancer, hypothalamic glioma, inflammatory breast cancer, intraocular melanoma, islet cell carcinoma, islet cell tumor, juvenile myelomonocytic leukemia, Kaposi sarcoma, Kaposi's sarcoma, kidney cancer, Klatskin tumor, Krukenberg tumor, laryngeal cancer, laryngeal cancer, lentigo maligna melanoma, leukemia, lip and oral cavity cancer, liposarcoma, lung cancer, luteoma, lymphangioma, lymphangiosarcoma, lymphoepithelioma, lymphoid leukemia, lymphoma, macroglobulinemia, malignant fibrous histiocytoma, malignant fibrous histiocytoma, malignant fibrous histiocytoma of bone, malignant glioma, malignant mesothelioma, malignant peripheral nerve sheath tumor, malignant rhabdoid tumor, malignant triton tumor, malt lymphoma, mantle cell lymphoma, mast cell leukemia, mediastinal germ cell tumor, mediastinal tumor, medullary thyroid cancer, medulloblastoma, medulloepithelioma, melanoma, meningioma, merkel cell carcinoma, mesothelioma, metastatic squamous neck cancer with occult primary, metastatic urothelial carcinoma, mixed mullerian tumor, monocytic leukemia, mouth cancer, mucinous tumor, multiple endocrine neoplasia syndrome, multiple myeloma, multiple myeloma, mycosis fungoides, mycosis fungoides, myelodysplastic disease, myelodysplastic syndromes, myeloid leukemia, myeloid sarcoma, myeloproliferative disease, myxoma, nasal cavity cancer, nasopharyngeal cancer, nasopharyngeal carcinoma, neoplasm, neurinoma, neuroblastoma, neuroblastoma, neurofibroma, neuroma, nodular melanoma, non-Hodgkin lymphoma, nonmelanoma skin cancer, non-small cell lung cancer, ocular oncology, oligoastrocytoma, oligodendroglioma, oncocytoma, optic nerve sheath meningioma, oral cancer, oral cancer, oropharyngeal cancer, osteosarcoma, ovarian cancer, ovarian cancer, ovarian epithelial cancer, ovarian germ cell tumor, ovarian low malignant potential tumor, Paget's disease of the breast, pancoast tumor, pancreatic cancer, pancreatic cancer, papillary thyroid cancer, papillomatosis, paraganglioma, paranasal sinus cancer, parathyroid cancer, penile cancer, perivascular epithelioid cell tumor, pharyngeal cancer, pheochromocytoma, pineal parenchymal tumor of intermediate differentiation, pineoblastoma, pituicytoma, pituitary adenoma, pituitary tumor, plasma cell neoplasm, pleuropulmonary blastoma, polyembryoma, precursor t-lymphoblastic lymphoma, primary central nervous system lymphoma, primary effusion lymphoma, primary hepatocellular cancer, primary liver cancer, primary peritoneal cancer, primitive neuroectodermal tumor, prostate cancer, pseudomyxoma peritonei, rectal cancer, renal cell carcinoma, respiratory tract carcinoma involving the nut gene on chromosome 15, retinoblastoma, rhabdomyoma, rhabdomyosarcoma, Richter's transformation, sacrococcygeal teratoma, salivary gland cancer, sarcoma, schwannomatosis, sebaceous gland carcinoma, secondary neoplasm, seminoma, serous tumor, Sertoli-Leydig cell tumor, sex cord-stromal tumor, sezary syndrome, signet ring cell carcinoma, skin cancer, small blue round cell tumor, small cell carcinoma, small cell lung cancer, small cell lymphoma, small intestine cancer, soft tissue sarcoma, somatostatinoma, soot wart, spinal cord tumor, spinal tumor, splenic marginal zone lymphoma, squamous cell carcinoma, stomach cancer, superficial spreading melanoma, supratentorial primitive neuroectodermal tumor, surface epithelial-stromal tumor, synovial sarcoma, T-cell acute lymphoblastic leukemia, T-cell large granular lymphocyte leukemia, T-cell leukemia, T-cell lymphoma, T-cell prolymphocytic leukemia, teratoma, terminal lymphatic cancer, testicular cancer, thecoma, throat cancer, thymic carcinoma, thymoma, thyroid cancer, transitional cell cancer of renal pelvis and ureter, transitional cell carcinoma, urachal cancer, urethral cancer, urogenital neoplasm, uterine sarcoma, uveal melanoma, vaginal cancer, Verner-Morrison syndrome, verrucous carcinoma, visual pathway glioma, vulvar cancer, Waldenstrom's macroglobulinemia, Warthin's tumor, Wilms' tumor.

The patient or subject to be treated can be a human patient with a disease or disorder described herein. In some embodiments, the subject is a cancer patient. In some embodiments, the subject is a virus-infected patient (e.g., a CMV-infected or HIV infected patient). In some embodiments, the subject has and/or is being treated for a cancer or tumor.

In some embodiments, the subject is administered one dose during the treatment. In some embodiments, the subject is administered at least two doses during the treatment. In some embodiments, the subject receives an initial dose and one or more (e.g., 2, 3, 4, or 5) subsequent administrations. In one embodiment, the one or more subsequent administrations are administered less than 15 days (e.g., 14, 13, 12, 11, 10, 9, 8, 7, 6, 5, 4, 3, or 2 days) after the previous administration after the previous administration. A person of ordinary skill in the art would be able to adjust and optimize the doses as necessary and appropriate.

In some embodiments, one or more additional therapeutic agents can be administered to the subject. In some embodiments, the antibody conjugations and pharmaceutical compositions described herein are used as medicament for the treatment of diseases as an adjunct to, or in conjunction with, other established therapies normally used in the treatment of such diseases. The additional therapeutic agent can be administered prior to, concurrently with, or after the administration of the antibody conjugations or pharmaceutical population. The additional therapeutic agent can be selected from the group consisting of an immunotherapeutic agent, a cytotoxic agent, a growth inhibitory agent, a radiation therapy agent, an anti-angiogenic agent, or any combination thereof. The additional therapeutic agent can be an immunotherapeutic agent, which can act on a target within the subject's body. In some embodiments, the additional therapeutic agent is an antibody targeting a tumor antigen.

The administration of the compositions can be carried out in any convenient manner. The antibody conjugations and pharmaceutical compositions described herein can be administered to a subject transarterially, subcutaneously, intradermally, intratumorally, intranodally, intramedullary, intramuscularly, by intravenous injection, or intraperitoneally, e.g., by intradermal or subcutaneous injection. The antibody conjugations or compositions can be injected directly into a tumor, lymph node, or site of infection.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Methods of Production

Provided herein are also methods of manufacturing the antibody conjugate described herein, including procedure for linking an antibody or antigen-binding fragment thereof with (A) a first functional unit and (B) a second functional unit;
wherein, the procedure (A) for linking the antibody or antigen-binding fragment thereof with the first functional unit includes the following steps (a1) to (a3):
(a1) providing the antibody or antigen-binding fragment thereof, wherein the antibody or antigen-binding fragment thereof includes:
   a light chain including on its surface a first amino acid residue and a second amino acid residue, and the first light chain is a kappa light chain; and
   heavy chains or heavy chain fragments; wherein each of the heavy chains or heavy chain fragments includes on its surface at least a third amino acid residue;
(a2) in a first conjugation buffer, contacting the antibody or antigen-binding fragment thereof with a first reducing agent, to expose first free thiols in the first amino acid residue;
(a3) in the first conjugation buffer, linking the antibody or antigen-binding fragment thereof with a first functional unit via a first covalent bond between the first amino acid residue and a TR residue of the first functional unit, wherein the TR residue includes a sulfhydryl-reactive group; and
wherein, the procedure (B) for linking the antibody or antigen-binding fragment thereof with the second functional unit includes the following steps (b1) to (b2):
(b1) in a second conjugation buffer, contacting the antibody or antigen-binding fragment thereof with an AAR linker, to generate a linker residue including a BL residue which covalently bonded to the second amino acid residue;
(b2) in a third conjugation buffer, reacting with a molecule including a second functional moiety, to generate a second covalent bond between the linker residue and the molecule.

In some embodiments, the step (b2) includes substeps:
(b21) in the second conjugation buffer, reducing a disulfide bond in the linker residue with a second reducing agent, to expose second free thiols; and
(b22) in a third conjugation buffer, reacting the second free thiols with a sulfhydryl group, an acrylate, an alkyl halide, an aziridine, a maleimidocaproyl group, a vinyl sulfone, a pyridyl disulfide, or other electrophile in the molecule including the second functional moiety, to link the second amino acid residue and the molecule including the second functional moiety via the reaction between the second free thiol and the molecule including the second functional moiety.

In some embodiments, the kappa light chain includes a lysine residue corresponding to a position 188 (Lys188) or 190 (Lys190), or wherein the antibody or antigen-binding fragment thereof is an immunoglobulin G (IgG) antibody.

In some embodiments, the AAR linker includes a structure represented by the following formula:

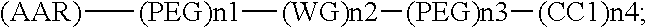

wherein,
-(PEG)n1- represents —(CH₂—CH₂—O)n1-, and n1 is an integer from 0 to 30;
W is a functional linker residue including —C(═O)—(CH₂)n8-S-(Succinimid-3-yl-N)—, —C(═O)—(CH₂)n10-, maleimidocaproic acid, an alkyne, a 3-(pyridin-2-yldisulfanyl) propanoate (PDP) residue, an azide, an alkene, an ester, a sulfhydryl group, a hydroxyl group, a thiol, an aldehyde, a ketone, a photoreactive moieties, —S—, or —S—R³—, and n2 is 0 or 1, wherein n8 is an integer from 0 to 10, and n10 is an integer from 0 to 10;
-(Succinimid-3-yl-N)— has a structure represented by the following formula:

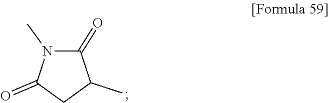

R³ is selected from the group consisting of C₁₋₆ alkyl, C₁₋₆ alkoxy, halo, halo(C₁₋₆)alkyl, hydroxy, C₁₋₆ alkylthio, C₁₋₆ alkoxycarbonyl, cyano, nitro, amino, mono- or di-C₁₋₆ alkylamino, C₁₋₆ acylamino, C₁₋₆ acyl, carboxy, C₁₋₆ alkoxycarbonyl, C₁₋₆ alkylsulfonyl, C₁₋₆ alkylsulfonamido, and phenyl or substituted phenyl;
-(PEG)n3- represents —(CH₂—CH₂—O)n3-, and n3 is an integer from 0 to 30;
CC1 is a reaction moiety of a bioorthogonal pair; and
AAR has a structure represented by one of the following formula:

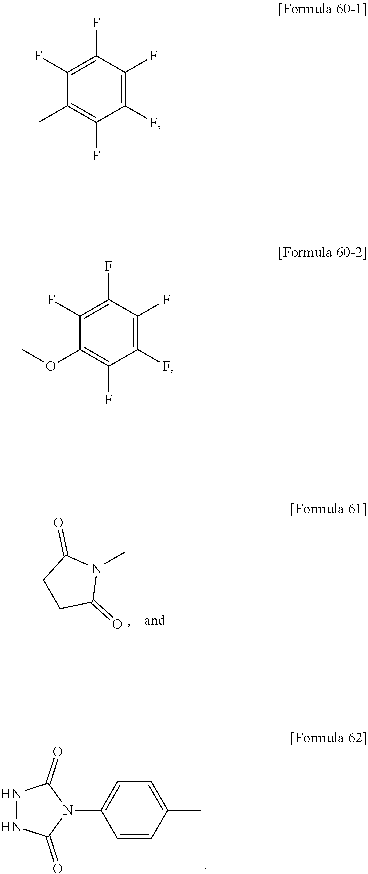

In some embodiments, the molecule including the second functional moiety is represented by the following structure:

—(CC2)n4-(PEG)n5-(NH—C(═O)CH₂—OCH₂)n19-(C(═O))n14-(AA)n6-(Spacer)n7-D2;
wherein,
   CC2 is a reaction moiety of the bioorthogonal pair; and n4 is 0 or 1;
   -(PEG)n5- represents —(CH₂—CH₂—O)n5-, and n5 is an integer from 0 to 30;
   -(AA)n6- represents n6 amino acid residues selected from the group consisting of the basic amino acid, the neutral amino acid, the hydrophobic amino acid, the polar acidic amino acid, and any combination thereof, wherein n6 is an integer from 0 to 30;
   Spacer is a self-immolative molecule connecting an oxygen or nitrogen of the second functional moiety, wherein the spacer is hemiaminal-related molecule, heterocyclic molecule, Mannich-type molecule, PABC, an aforementioned molecule substituted with a hydrophobicity-masking entity, or other spacer, and n7 is 0 or 1;
   n14 is 0 or 1, n19 is 0 or 1; and
   D2 is a second functional moiety.

In some embodiments, the n6 amino acid residues are linked in sequence.

In some embodiments, the first conjugation buffer can be a phosphate buffer, an acetate buffer, a Tris buffer, a citrate buffer, a sodium chloride solution, an EDTA buffer, or an amino acid-containing buffer.

In some embodiments, the AAR linker can be a PFP-based linker. The term “PFP-based linker” as used herein refers to any compound or moiety that includes at least one pentafluorophenol (PFP) functional group and is structurally or functionally adapted to covalently connect or bridge two molecules (for example, adapted to covalently connect or bridge a functional (bio)molecule with a molecular tag), thereby forming a conjugate.

In some embodiments, the PFP-based linker includes a disulfide bond or an azide functional group.

In some embodiments, the PFP-based linker can be a propargyl-SS-PFP ester, a 2,3,4,5,6-pentafluorophenyl 4-(pyridin-2-yldisulfanyl) butanoate (PDB-PFP), a 2,3,4,5,6-pentafluorophenyl 3-(pyridin-2-yldisulfanyl)propanoate (PDP-PFP), a perfluorophenyl 3-(pyridin-2-yldisulfanyl)propanoate, perfluorophenyl 4-methyl-4-(pyridine-2-yldisulfanyl)pentanoate, or a perfluorophenyl 4-(pyridin-2-yldisulfanyl)butanoate.

In some embodiments, the second conjugation buffer can be a phosphate buffer, an acetate buffer, a Tris buffer, a citrate buffer, a sodium chloride solution, an EDTA buffer, or an amino acid-containing buffer.

In some embodiments, the third conjugation buffer can be a phosphate buffer, an acetate buffer, a Tris buffer, a citrate buffer, a sodium chloride solution, an EDTA buffer, or an amino acid-containing buffer.

In some embodiments, the first reducing agent and/or the second reducing agent can be beta-mercaptoethanol (BME), dithiothreitol (DTT), tris(2-carboxyethyl)phosphine (TCEP), 2-Mercaptoethylamine (2-MEA), or thiol-based reducing agents.

In some embodiments, a molecular weight of each one of the first functional unit and the second functional unit is 0.5 to 20 kDa.

In some embodiments, (AAR)-(PEG)n1-(W)n2-(PEG)n3-(CC1)n4- has a structure represented by the following formula:

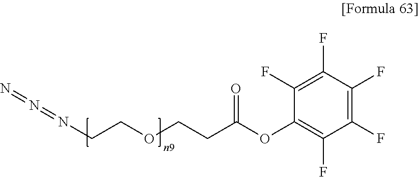

and wherein n9 is an integer from 0 to 2.

### EXAMPLES

The examples provided below are for purposes of illustration only, which are not intended to be limiting unless otherwise specified. Thus, the invention should in no way be construed as being limited to the following examples, but rather, should be construed to encompass any and all variations which become evident as a result of the teaching provided herein.

Exemplary genes, protein, and polypeptides are described herein with reference to GenBank numbers, GI numbers and/or SEQ ID NOs. It is understood that one skilled in the art can readily identify homologous sequences by reference to sequence sources, including but not limited to GenBank (ncbi.nlm.nih.gov/genbank/), Protein (https://www.ncbi.nlm.nih.gov/protein), and EMBL (embl.org/).

### Embodiment 1: Atezolizumab-Govitecan (Cysteine and Lysine Based Conjugation)

### Embodiment 1-1: Preparation of Atezolizumab-Govitecan (DAR 10)

Please refer to FIG. 3, which illustrates the process of manufacturing a DAR10 Atezolizumab-govitecan via procedure (A) conjugating 8 numbers of the first functional moieties (govitecan) and procedure (B) conjugating 2 numbers of the second functional moiety (govitecan) to an antibody (Atezolizumab), wherein the first functional moieties are linked to the cysteine residue on the surface of the light chain and the heavy chain of the antibody via the first linker residue, and the second functional moieties are linked to the lysine residue on the surface of the light chain of the antibody via the second linker residue.

Atezolizumab is an IgG1 antibody and includes heavy chains and kappa light chains, wherein the kappa light chains comprise a lysine residue corresponding to a position 188 (Lys188) or 190 (Lys190). The Sequence of its light chains could be found in NCBI with GI number 1248694005 or 1233034598 but not limited to (please refer to https://www.ncbi.nlm.nih.gov/protein/5XXY_L or https://www.ncbi.nlm.nih.gov/protein/5X8L_O). The Sequence of its heavy chains could be found in NCBI with GI number 1248694004 or 1233034599 but not limited to (please refer to https://www.ncbi.nlm.nih.gov/protein/5XXY_H or https://www.ncbi.nlm.nih.gov/protein/5X8L_J).

The methods of manufacturing the DAR10 Atezolizumab-govitecan includes at least the procedures for linking an Atezolizumab (antibody) with (A-1) a first functional unit and (B-1) a second functional unit;
wherein, the procedure (A-1) for linking the Atezolizumab (antibody) with the first functional unit includes at least the following steps (a1-1) to (a3-1):
(a1-1) the Atezolizumab (commercially available anti-PDL1 antibody) was provided in a conjugation buffer, wherein the Atezolizumab includes:
   a first light chain and a second light chain; each of the light chains includes on its surface a cysteine residue (a first amino acid residue) and a lysine residue (Lys188 or Lys 190, a second amino acid residue), and the first light chain is a kappa light chain; and
   heavy chains; wherein each of the heavy chains includes on its surface at least three cysteine residues (third amino acid residues);
(a2-1) Tris(2-carboxyethyl)phosphine (TCEP, a first reducing agent) was added, to dissociate interchain disulfide bonds of the Atezolizumab (antibody) and therefore expose first free thiols in cysteine residues (the first amino acid residues and the third amino acid residues);
(a3-1) a first functional unit including a linker residue of -TR-(Y)m1-(PEG)m2-(Z)m3-(AA)m4-(Spacer)m5- and govitecan (a first functional moiety) was added, to link the cysteine residues (the first amino acid residues and the third amino acid residues) and the first functional unit via a covalent bond between the first free thiols in cysteine residues and the TR residue in the first functional unit, wherein the first functional unit has a structure represented by the following formula:

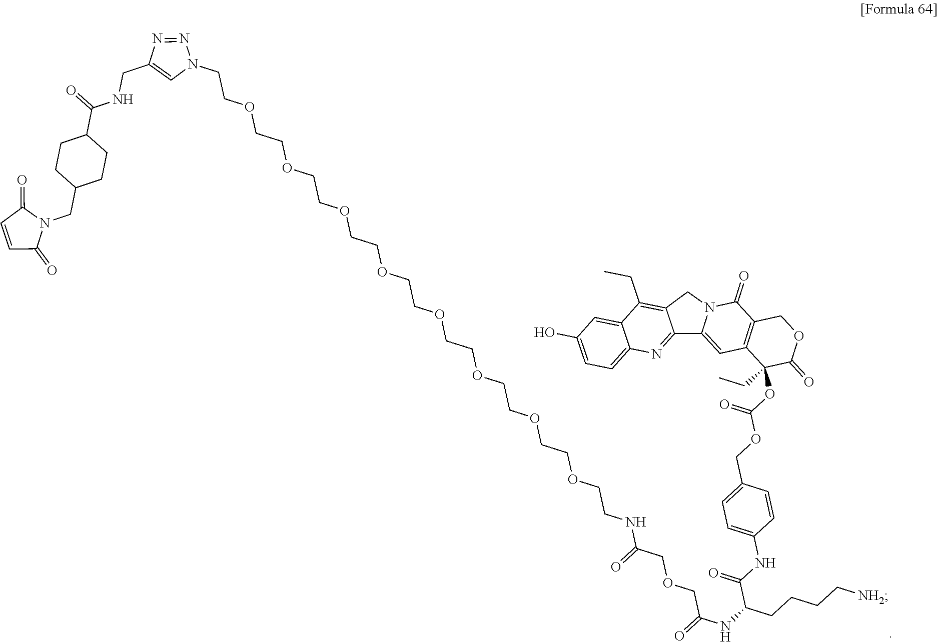 [SMILES: CCc1c2c(nc3ccc(O)cc13)-c1cc3c(c(=O)n1C2)COC(=O)[C@@]3(CC)OC(=O)OCc1ccc(NC(=O)[C@H](CCCCN)NC(=O)COCC(=O)NCCOCCOCCOCCOCCOCCOCCOCCOCCn2cc(CNC(=O)C3CCC(CN4C(=O)C=CC4=O)CC3)nn2)cc1]

and
wherein, the procedure (B-1) for linking Atezolizumab (antibody) with the second functional unit includes the following steps (b1-1) to (b2-1):
(b1-1) an AAR linker having a structure represented by the formula (AAR)-(PEG)n1-(W)n2-(PEG)n3-(CC1)n4 was added at 4° C., to generate a linker residue including a BL residue which covalently bonded to the lysine residues (the second amino acid residues) via the BL residue, wherein the AAR linker has a structure represented by the following formula:

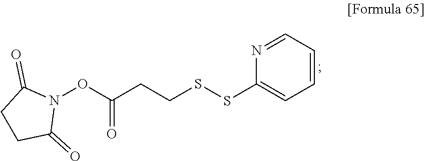 [SMILES: O=C(CCSSc1ccccn1)ON1C(=O)CCC1=O]

the reaction mixture was then placed in the centrifuge filtration tube with membrane at 30 kDa cut-off for washing steps. The washing steps included (1) centrifuge the centrifuge filtration tube containing AAR linker-modified atezolizumab to remove unreacted AAR linker and (2) wash the retained AAR linker-conjugated atezolizumab with 1× volume of washing buffer to assure removing unreacted AAR linker; and
(b2-1) a molecule including govitecan (govitecan is the second functional moiety) was added, to link the lysine residues (the second amino acid residues) and the molecules including govitecan (govitecan is the second functional moiety) via the reaction between the linker residue and the molecule including govitecan; wherein the molecule including govitecan has a structure represented by the following formula.

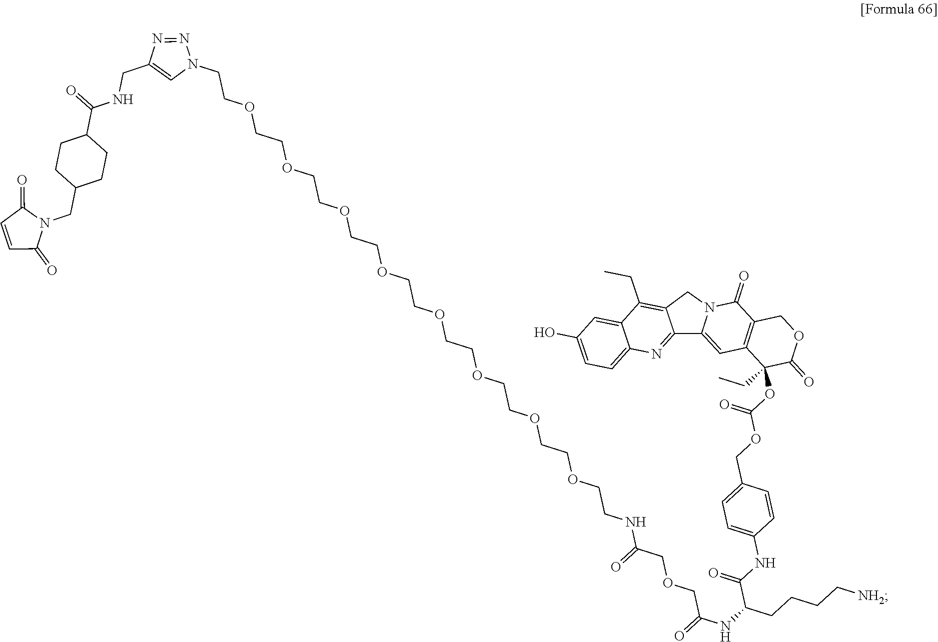 [SMILES: CCc1c2c(nc3ccc(O)cc13)-c1cc3c(c(=O)n1C2)COC(=O)[C@@]3(CC)OC(=O)OCc1ccc(NC(=O)[C@H](CCCCN)NC(=O)COCC(=O)NCCOCCOCCOCCOCCOCCOCCOCCOCCn2cc(CNC(=O)C3CCC(CN4C(=O)C=CC4=O)CC3)nn2)cc1]

and wherein the step (b2-1) includes substeps:
(b21-1) Tris(2-carboxyethyl)phosphine (TCEP, a second reducing agent) was added at room temperature, to reduce a disulfide bond in the linker residue and therefore to expose second free thiols in the linker residue; and
(b22-1) the molecule including govitecan (govitecan is the second functional moiety) was added, to react the second free thiols with the molecule including govitecan, and therefore to link the lysine residues (the second amino acid residues) and the molecules including govitecan (govitecan is the second functional moiety) via the reaction between the second free thiol and the molecule including govitecan.

The reaction mixture was then washed as previously described.

The concentration of retained atezolizumab-govitecan at DAR 10 was then quantified by the BCA quantification method.

The conjugation buffer can be selected from the group consisting of a phosphate buffer, an acetate buffer, a Tris buffer, a citrate buffer, a sodium chloride solution, an EDTA buffer, or an amino acid-containing buffer.

For the first functional unit including the linker residue of -TR-(Y)m1-(PEG)m2-(Z)m3-(AA)m4-(Spacer)m5- and govitecan (a first functional moiety) described in this Embodiment 1-1, the TR residue is a maleimide residue (or -(Succinimid-3-yl-N)—) which includes a sulfhydryl-reactive group, Y is a Y molecule residue including an alkyl group or a cycloalkyl group having a structure represented by the following formula:

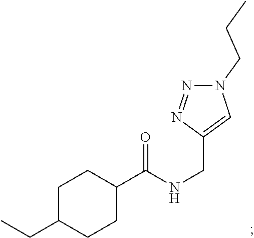 [SMILES: CCCn1cc(CNC(=O)C2CCC(CC)CC2)nn1]

Z residue is a Z molecule residue having a structure represented by the following formula:

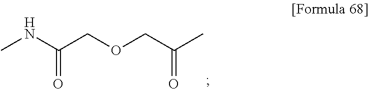

-(AA)m4- is a lysine residue; and Spacer residue is a first linker unit connecting to the first functional moiety and having a structure represented by the following formula:

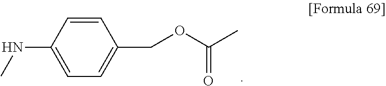

For the AAR linker having the structure represented by the formula (AAR)-(PEG)n1-(W)n2-(PEG)n3-(CC1)n4 described in this Embodiment 1-1, W is a functional linker residue including —C(═O)—(CH₂)—(CH₂)—; CC1 is a reaction moiety of a bioorthogonal pair having a structure represented by the following formula:

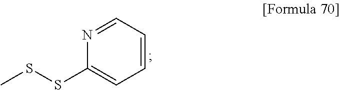

and
AAR has a structure represented by the following formula:

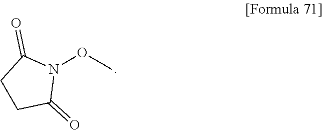

The product in this Embodiment 1-1 includes a second functional unit, wherein the second functional unit includes govitecan (a second functional moiety) and a linker residue (a second linker residue) of -(BL)-(PEG)n1-(W)n2-(PEG)n3-(CC1)n4-(CC2)n4-(PEG)n5-(NH—C(═O)CH₂—O—CH₂)n19-(C(═O))n14-(AA)n6-(Spacer)n7-; wherein
BL residue is a single bond,
W residue is —C(═O)—(CH₂)—(CH₂)—,
—CC1- residue is —S—,
—CC2- residue is (Succinimid-3-yl-N)—R⁴ having a structure represented by the following formula:

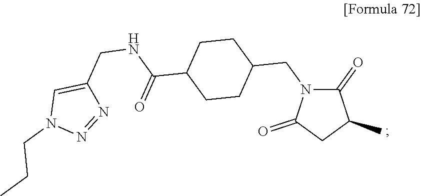 [SMILES: CCCn1cc(CNC(=O)C2CCC(CN3C(=O)C[C@H](C)C3=O)CC2)nn1]

-(AA)n6- is a lysine residue; and
   Spacer residue has a structure represented by the following formula

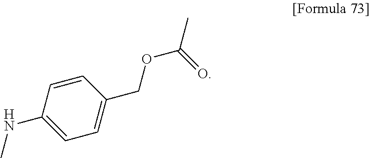

### Embodiment 1-2: Characterization of Atezolizumab-Govitecan (DAR 10)

RP-HPLC and mass spectrometry were applied for the atezolizumab-govitecan (DAR 10) prepared according to the process described in Embodiment 1-1.

There were two groups in this experiment:
Atezolizumab-govitecan (DAR8) was prepared according to the procedure (A) to conjugat 8 numbers of the first functional moieties (govitecan) described in FIG. 3; and
PDP-Atezolizumab-govitecan (DAR10) was prepared according to the procedure (A) to conjugate 8 numbers of the first functional moieties (govitecan) as well as procedure (B) to conjugate 2 numbers of the second functional moiety (govitecan) described in FIG. 3.

For the RP-HPLC analysis, 20 μg of reduced sample was injected into the RP-HPLC Agilent 1260 Infinity II System equipped with reverse phase column (AdvanceBioRP mAb Diphenyl, 4.6 mm×150 mm, 3.5 μm) and the signal was detected at 280 nm absorbance. The corresponding peaks of signals were presented as retention time.

Please refer to FIG. 4, which is the Reverse phase HPLC (RP-HPLC) results of the product prepared according to the process described in FIG. 3. The light chain of Atezolizumab-govitecan (DAR8, the light chain is represented by LC+1G) was at 9.269 minute, and light chain of PDP-Atezolizumab-govitecan (DAR10, the light chain is represented by LC+2G) shifted to about 10.6 minute, whereas the heavy chain showed no shift in PDP-Atezolizumab-govitecan (DAR10).

To further verify the light chain-specific conjugation, liquid chromatography equipped with mass spectrometry was applied. The peaks in the liquid chromatography sequentially injected into the mass spectrometry. The analysis revealed that the characterization and the mass peak of the light chain (represented by LC+2G) and the heavy chain (represented by HC+3G) of Atezolizumab-govitecan (DAR 10) could be confirmed.

### Embodiment 1-3: Binding Affinity of Atezolizumab-Govitecan (DAR 10)

Binding affinity experiment was applied for the atezolizumab-govitecan (DAR 10) prepared according to the process described in Embodiment 1-1. There were three groups in this experiment:
PDL1 mAb was the antibody “Atezolizumab” (DAR0);
PDP-PDL1 mAb-govitecan was the atezolizumab-govitecan (DAR10) prepared according to the procedure (A) to conjugate 8 numbers of the first functional moieties (govitecan) as well as procedure (B) to conjugate 2 numbers of the second functional moiety (govitecan) to Atezolizumab as described in FIG. 3 (DAR10); and
Isotype Ab was an isotype control antibody that lacks specificity to the target (PDL1) and therefore acts as negative controls.

A 96-well white plate was coated with His-tagged human PDL1 recombinant protein and then blocked with 5% skim milk. The blocked His-tagged human PDL1 recombinant protein-coated white plate was then incubated with atezolizumab, atezolizumab-govitecan (DAR10), or the isotype control antibody at different concentrations at 37° C. for 1 hour. The unbound atezolizumab, atezolizumab-govitecan (DAR10), or the isotype control antibody was washed away with PBST. The bound atezolizumab and atezolizumab-govitecan was detected by HRP-conjugated anti-human IgG and the developed color signal was recorded at 450 nm.

Please refer to FIG. 5, which is the binding affinity results of the product prepared according to the process described in FIG. 3. The results revealed that there was no significant difference of signal detection between atezolizumab and atezolizumab-govitecan (DAR10) among the concentration range, suggesting the conjugation of 10 numbers of govitecan elicited no impact on the PDL1 binding of atezolizumab. This is an unexpected result. That is, a person skilled in the art would expect that conjugation of 2 numbers of 1.5 kDa linker-payload to a 25 kDa light chain of the antibody should results in steric hindrance to significantly impair antigen-binding affinity. However, as depicted in FIG. 5 of the present disclosure, conjugation of two govitecan molecules to each of the light chains and conjugation of three govitecan molecules to each of the heavy chains elicited no impact on the PDL1 binding of atezolizumab, which constitutes to an unexpected result.

Moreover, as summarized in Table 1 based on the binding affinity result shown in FIG. 5, the EC50 of atezolizumab-govitecan (DAR10) is 0.75 nM, which is less than 2 times of EC50 of atezolizumab (0.75 is less than 2×0.55).

TABLE 1

| | EC₅₀ | | |
|---|---|---|---|
| (N = 3) | ng/mL | nM | R² |
| PDL1 mAb | 82.68 | 0.55 | 0.9988 |
| PDL1 mAb-govitecan | 111.80 | 0.75 | 0.9984 |
| Isotype Ab | >4000 | >26.67 | — |

### Embodiment 1-4: Cytotoxicity of Atezolizumab-Govitecan (DAR 10)

Cytotoxicity assay was applied for the atezolizumab-govitecan (DAR 10) prepared according to the process described in Embodiment 1-1. There were four groups in this experiment:
PDL1 mAb was the antibody “Atezolizumab” (DAR0).
PDL1 mab-govitecan was atezolizumab-govitecan (DAR8) prepared according to the procedure (A) to conjugate 8 numbers of the first functional moieties (govitecan) to Atezolizumab as described in FIG. 3 (DAR8).
PDP-PDL1 mab-govitecan was the atezolizumab-govitecan (DAR10) prepared according to the procedure (A) to conjugate 8 numbers of the first functional moieties (govitecan) and procedure (B) to conjugate 2 numbers of the second functional moiety (govitecan) to Atezolizumab as described in FIG. 3 (DAR10).
SN-38 was the payload of govitecan is used as positive control of cytotoxicity.

One thousand human triple-negative breast cancer MDA-MB-231 cells were seeded into each well of a white flat-bottom 96-well plate for an overnight incubation and then treated with atezolizumab, atezolizumab-govitecan (DAR8), atezolizumab-govitecan (DAR10) and SN-38 at concentration 0.01, 0.05, 0.2, 1, 2, 5, 20, 50, 100, 500 nM for 6 days. Each condition was performed in triplicate. On the harvesting day, the number of viable cells in each well were measured according to the CellTiter-Glo™ 2.0 Assay process.

Please refer to FIG. 6A and FIG. 6B, which are the cytotoxicity results indicating that the product prepared according to the process described in FIG. 3 exhibits cytotoxicity against human triple-negative breast cancer cell line MDA-MB-231. The results in FIG. 6B revealed that atezolizumab-govitecan (DAR10) had better cytotoxicity than atezolizumab-govitecan (DAR8), atezolizumab and SN-38 (p<0.05), suggesting high-DAR ADCs exhibit higher cytotoxicity. Moreover, FIG. 6B revealed that compared with atezolizumab, atezolizumab-govitecan including 8 number of govitecan (DAR8) only killed 25% of cancer cells (100%−75%=25%), atezolizumab-govitecan including 10 number of govitecan in (DAR10) could kill 50% of cancer cells (100%−50%=50%). In other words, the antibody drug conjugate prepared according to the present disclosure exhibits enhanced cytotoxicity, demonstrating an unexpected result.

### Embodiment 2: Atezolizumab-Deruxtecan-vcMMAE (Cysteine and Lysine Based Conjugation)

### Embodiment 2-1: Preparation of Atezolizumab-Deruxtecan-vcMMAE (DAR 10)

Please refer to FIG. 7, which illustrates the process of manufacturing a DAR10 Atezolizumab-deruxtecan-vcMMAE via procedure (A) conjugating 8 numbers of the first functional moieties (deruxtecan) and procedure (B) conjugating 2 numbers of the second functional moiety (MMAE) to an antibody (Atezolizumab), wherein the first functional moieties are linked to the cysteine residue on the surface of the light chain and the heavy chain of the antibody via the first linker residue, and the second functional moieties are linked to the lysine residue on the surface of the light chain of the antibody via the second linker residue.

The methods of manufacturing the DAR10 Atezolizumab-deruxtecan-vcMMAE includes at least the procedures for linking an Atezolizumab (antibody) with (A-2) a first functional unit and (B-2) a second functional unit;
wherein, the procedure (A-2) for linking the Atezolizumab (antibody) with the first functional unit includes at least the following steps (a1-2) to (a3-2):
(a1-2) the Atezolizumab (commercially available anti-PDL1 antibody) was provided in a conjugation buffer, wherein the Atezolizumab includes:
   a first light chain and a second light chain; each of the light chains includes on its surface a cysteine residue (a first amino acid residue) and a lysine residue (Lys188 or Lys 190, a second amino acid residue), and the first light chain is a kappa light chain; and
   heavy chains, wherein each of the heavy chains includes on its surface at least three cysteine residues (third amino acid residues);
(a2-2) Tris(2-carboxyethyl)phosphine (TCEP, a first reducing agent) was added, to dissociate interchain disulfide bonds of the Atezolizumab (antibody) and therefore expose first free thiols in cysteine residues (the first amino acid residues and the third amino acid residues);
(a3-2) a first functional unit including a linker residue of -TR-(Y)m1-(PEG)m2-(Z)m3-(AA)m4-(Spacer)m5- and deruxtecan (a first functional moiety) was added, to link the cysteine residues (the first amino acid residues and the third amino acid residues) and the first functional unit via a covalent bond between the first free thiols in cysteine residues and the TR residue in the first functional unit, wherein the first functional unit has a structure represented by the following formula:

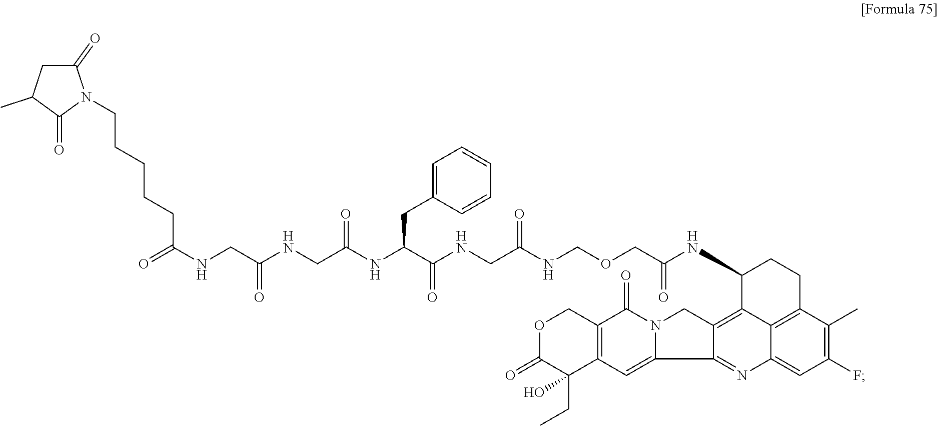 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)CC(C)C1=O)CC3]

and
wherein, the procedure (B-2) for linking Atezolizumab (antibody) with the second functional unit includes the following steps (b1-2) to (b2-2):
(b1-2) an AAR linker having a structure represented by the formula (AAR)-(PEG)n1-(W)n2-(PEG)n3-(CC1)n4 was added at 4° C., to generate a linker residue including a BL residue which covalently bonded to the lysine residues (the second amino acid residues) via the BL residue, wherein the AAR linker has a structure represented by the following formula:

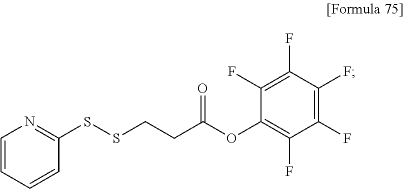 [SMILES: O=C(CCSSc1ccccn1)Oc1c(F)c(F)c(F)c(F)c1F]

the rection mixture was then placed in the centrifuge filtration tube with membrane at 30 kDa cut-off for washing steps. The washing steps included (1) centrifuge the centrifuge filtration tube containing AAR linker-modified atezolizumab to remove unreacted AAR linker and (2) wash the retained AAR linker-conjugated atezolizumab with 1× volume of washing buffer to assure removing unreacted AAR linker; and
(b2-2) a molecule including MMAE (MMAE is the second functional moiety) was added, to link the lysine residues (the second amino acid residues) and the molecules including MMAE via the reaction between the linker residue and the molecule including MMAE; wherein the molecule including MMAE has a structure represented by the following formula:

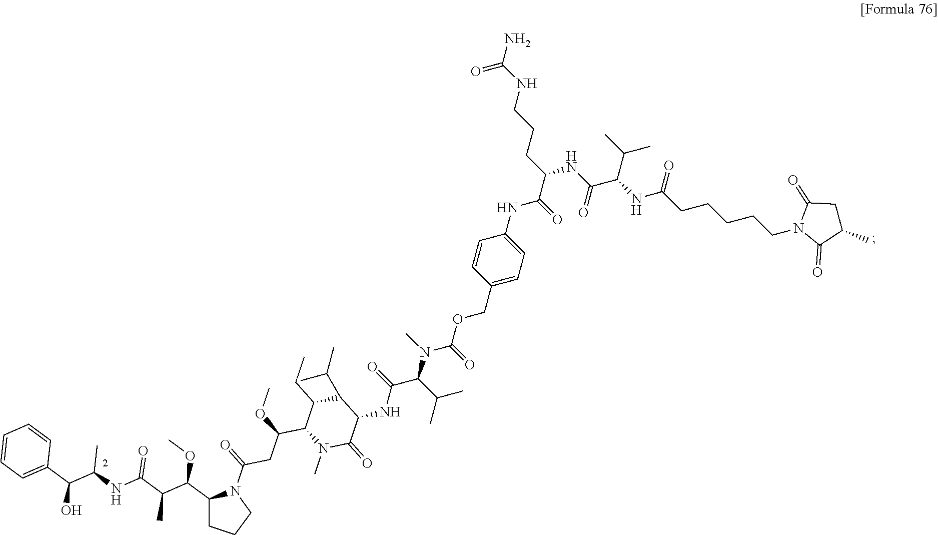

and wherein the step (b2-2) includes substeps.
(b21-2) Tris(2-carboxyethyl)phosphine (TCEP, a second reducing agent) was added at room temperature, to reduce a disulfide bond in the linker residue and therefore to expose second free thiols in the linker residue; and
(b22-2) the molecule including MMAE (MMAE is the second functional moiety) was added, to react the second free thiols with the molecule including MMAE, and therefore to link the lysine residues (the second amino acid residues) and the molecules including MMAE (MMAE is the second functional moiety) via the reaction between the second free thiol and the molecule including MMAE.

The reaction mixture was then washed as previously described.

The concentration of retained atezolizumab-deruxtecan-vcMMAE at DAR 10 was then quantified by the BCA quantification method.

The conjugation buffer can be selected from the group consisting of a phosphate buffer, an acetate buffer, a Tris buffer, a citrate buffer, a sodium chloride solution, an EDTA buffer, or an amino acid-containing buffer.

For the first functional unit including the linker residue of -TR-(Y)m1-(PEG)m2-(Z)m3-(AA)m4-(Spacer)m5- and deruxtecan (a first functional moiety) described in this Embodiment 2-1, the TR residue is a maleimide residue (or -(Succinimid-3-yl-N)—) which includes a sulfhydryl-reactive group, Y is a Y molecule residue including an alkyl group having a structure represented by the following formula:

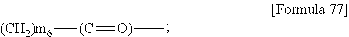

Z residue is an Z molecule residue having a structure represented by the following formula:

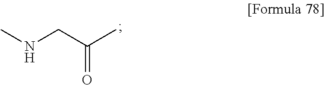

-(AA)m4- is a peptide linkage made by linking a phenylalanine residue and a glycine residue; and Spacer residue is a first linker unit connecting to the first functional moiety and having a structure represented by the following formula:

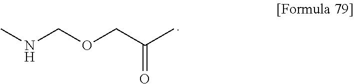

For the AAR linker having the structure represented by the formula (AAR)-(PEG)n1-(W)n2-(PEG)n3-(CC1)n4 described in this Embodiment 2-1, W is a functional linker residue including —C(═O)—(CH₂)—(CH₂)—; CC1 is a reaction moiety of a bioorthogonal pair having a structure represented by the following formula:

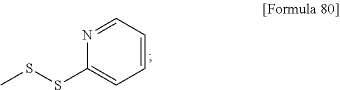

and
AAR has a structure represented by the following formula:

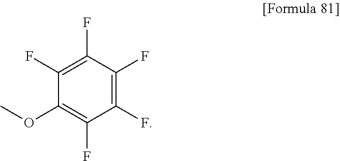

The product in this Embodiment 2-1 includes a second functional unit, wherein the second functional unit includes MMAE (a second functional moiety) and a linker residue (a second linker residue) of -(BL)-(PEG)n1-(W)n2-(PEG)n3-(CC1)n4-(CC2)n4-(PEG)n5-(NH—C(═O)CH₂—O—CH₂)n19-(C(═O))n14-(AA)n6-(Spacer)n7-; wherein
BL residue is a single bond,
W residue is —C(═O)—(CH₂)—(CH₂)—,
—CC1- residue is —S—,
—CC2- residue is (Succinimid-3-yl-N)—R⁴ having a structure represented by the following formula:

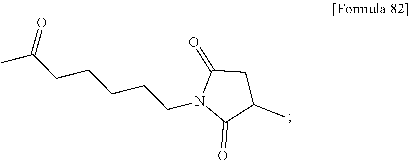 [SMILES: CC(=O)CCCCCN1C(=O)CC(C)C1=O]

-(AA)n6- is a peptide linkage made by linking a valine residue and a citrulline residue; and Spacer residue has a structure represented by the following formula

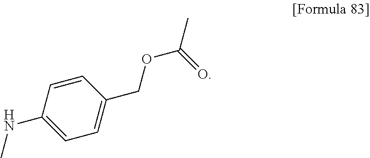

### Embodiment 2-2: Characterization of Atezolizumab-Deruxtecan-vcMMAE (DAR 10)

RP-HPLC and mass spectrometry were applied for the atezolizumab-deruxtecan-vcMMAE (DAR 10) prepared according to the process described in Embodiment 2-1.

There were three groups in this experiment:
Atezolizumab was the antibody (DAR0).
Atezolizumab-GGFG-DXd was prepared according to the procedure (A) to conjugate 8 numbers of the first functional moieties (deruxtecan) described in FIG. 7 (DAR8).

Atezolizumab-GGFG-DXd-vcMMAE was prepared according to the procedure (A) to conjugate 8 numbers of the first functional moieties (deruxtecan) and procedure (B) to conjugate 2 numbers of the second functional moiety (MMAE) described in FIG. 7 (DAR10).

For the RP-HPLC analysis, 20 μg of reduced sample was injected into the RP-HPLC Agilent 1260 Infinity II System equipped with reverse phase column (AdvanceBioRP mAb Diphenyl, 4.6 mm×150 mm, 3.5 μm) and the signal was detected at 280 nm absorbance. The corresponding peaks of signals were presented as retention time.

Please refer to FIG. 8, which is the Reverse phase HPLC (RP-HPLC) results of the product prepared according to the process described in FIG. 7.

The light chain of Atezolizumab-GGFG-DXd (DAR8, the light chain is represented by LC+1D)) was at 11.129 minutes, and light chain of atezolizumab-deruxtecan-vcMMAE (DAR10, the light chain is represented by LC+1D+1V) shifted to about 13.654 minutes, whereas the heavy chain showed no shift in atezolizumab-deruxtecan-vcMMAE.

To further verify the light chain-specific conjugation, liquid chromatography equipped with mass spectrometry was applied. The peaks in the liquid chromatography sequentially injected into the mass spectrometry. The analysis revealed that the characterization and the mass peak of the light chain (represented by LC+1D+1V) and the heavy chain (represented by HC+3D) of atezolizumab-deruxtecan-vcMMAE (DAR 10) could be confirmed.

### Embodiment 2-3: Binding Affinity of Atezolizumab-Deruxtecan-vcMMAE (DAR 10)

Binding affinity experiment was applied for the atezolizumab-deruxtecan-vcMMAE (DAR 10) prepared according to the process described in Embodiment 2-1. There were two groups in this experiment:
PDL1 mAb was the antibody “Atezolizumab” (DAR0).
PDL1 mAb-DXd-MMAE was atezolizumab-deruxtecan-vcMMAE (DAR 10) prepared according to the procedure (A) to conjugate 8 numbers of the first functional moieties (deruxtecan) and procedure (B) to conjugate 2 numbers of the second functional moiety (MMAE) described in FIG. 7 (DAR10).

A 96-well white plate was coated with His-tagged human PDL1 recombinant protein and then blocked with 5% skim milk. The blocked His-tagged human PDL1 recombinant protein-coated white plate was then incubated with atezolizumab or atezolizumab-deruxtecan-vcMMAE (DAR 10) at different concentrations at 37° C. for 1 hour. The unbound atezolizumab or atezolizumab-deruxtecan-vcMMAE (DAR 10) was washed away with PBST. The bound atezolizumab and atezolizumab-deruxtecan-vcMMAE (DAR 10) was detected by HRP-conjugated anti-human IgG and the developed color signal was recorded at 450 nm.

Please refer to FIG. 9, which depicts the binding affinity results of the product prepared according to the process described in FIG. 7.

The results revealed that there was no significant difference of signal detection between atezolizumab and atezolizumab-deruxtecan-vcMMAE (DAR 10) among the concentration range, suggesting the conjugation of 8 numbers of deruxtecan and 2 numbers of vcMMAE elicited no impact on the PDL1 binding of atezolizumab. This is an unexpected result. That is, a person skilled in the art would expect that conjugation of 2 numbers of 1.5 kDa linker-payload to a 25 kDa light chain of the antibody should results in steric hindrance to significantly impair antigen-binding affinity. However, as revealed by FIG. 9, conjugation of one deruxtecan molecule and one vcMMAE molecule to each of the light chains and conjugation of three deruxtecan molecules to each of the heavy chains elicited no impact on the PDL1 binding of atezolizumab, demonstrating an unexpected result.

Moreover, please refer to Table 2. based on the binding affinity result shown in FIG. 9, the EC50 of atezolizumab-deruxtecan-vcMMAE (DAR 10) is 1.56 nM, which is less than 2 times of EC50 of atezolizumab (1.56 is less than 2×1.40).

TABLE 2

| | EC₅₀ | | |
|---|---|---|---|
| (N = 3) | ng/mL | nM | R² |
| Atezo-Deruxtecan-vcMMAE | 234.5 | 1.56 | 0.9997 |
| Atezolizumab | 210.2 | 1.40 | 0.9992 |

### Embodiment 2-4: Cytotoxicity of Atezolizumab-Deruxtecan-vcMMAE (DAR 10)

Cytotoxicity assay was applied for the atezolizumab-deruxtecan-vcMMAE (DAR 10) prepared according to the process described in Embodiment 2-1. There were four groups in this experiment:
Atezolizumab was the antibody (DAR0).
Atezolizumab-deruxtecan was prepared according to the procedure (A) to conjugate 8 numbers of the first functional moieties (deruxtecan) to Atezolizumab as described in FIG. 7 (DAR8).
Atezolizumab-deruxtecan-vcMMAE was prepared according to the procedure (A) to conjugate 8 numbers of the first functional moieties (deruxtecan) and procedure (B) to conjugate 2 numbers of the second functional moiety (MMAE) to Atezolizumab as described in FIG. 7 (DAR10).
DXd was first functional moieties.

One thousand human triple-negative breast cancer MDA-MB-231 cells were seeded into each well of a white flat-bottom 96-well plate for an overnight incubation and then treated with atezolizumab, Atezolizumab-deruxtecan (DAR8), Atezolizumab-deruxtecan-vcMMAE (DAR10) and DXd at concentration 0.01, 0.05, 0.2, 1, 2, 5, 20, 50, 100, 500 nM for 6 days. Each condition was performed in triplicate. On the harvesting day, the number of viable cells in each well were measured according to the CellTiter-Glo™ 2.0 Assay process.

The results revealed that atezolizumab-deruxtecan-vcMMAE (DAR 10) had better cytotoxicity than atezolizumab-deruxtecan (DAR8) and atezolizumab (DAR0).

### Embodiment 3: Trastuzumab-Exatecan-vcMMAE (Cysteine and Lysine Based Conjugation)

### Embodiment 3-1: Preparation of Trastuzumab-Exatecan-vcMMAE (DAR 10)

Please refer to FIG. 10, which illustrates the process of manufacturing a DAR10 Trastuzumab-exatecan-vcMMAE via procedure (A) conjugating 8 numbers of the first functional moieties (exatecan) and procedure (B) conjugating 2 numbers of the second functional moiety (MMAE) to an antibody (Trastuzumab), wherein the first functional moieties are linked to the cysteine residue on the surface of the light chain and the heavy chain of the antibody via the first linker residue, and the second functional moieties are linked to the lysine residue on the surface of the light chain of the antibody via the second linker residue.

Trastuzumab is an IgG1 antibody and includes heavy chains and kappa light chains, wherein the kappa light chains comprise a lysine residue corresponding to a position 188 (Lys188) or 190 (Lys190). The Sequence of its light chains could be found in NCBI with GI number 1653017363 or 2579823522 but not limited to (please refer to https://www.ncbi.nlm.nih.gov/protein/60GE_D or https://www.ncbi.nlm.nih.gov/protein/8Q6J_A). The Sequence of its heavy chains could be found in NCBI with GI number 1653017364 or 2579823523 but not limited to (please refer to https://www.ncbi.nlm.nih.gov/protein/60GE_E or https://www.ncbi.nlm.nih.gov/protein/8Q6J_B).

The methods of manufacturing the DAR10 Trastuzumab-exatecan-vcMMAE includes at least the procedures for linking an Trastuzumab (antibody) with (A-3) a first functional unit and (B-3) a second functional unit;
wherein, the procedure (A-3) for linking the Trastuzumab (antibody) with the first functional unit includes at least the following steps (a1-3) to (a3-3):
(a1-3) the Trastuzumab (commercially available anti-HER2 antibody) was provided in a conjugation buffer, wherein the Trastuzumab includes:
   two light chains; each of the light chains includes on its surface a cysteine residue (a first amino acid residue) and a lysine residue (Lys188 or Lys 190, a second amino acid residue), and the first light chain is a kappa light chain; and heavy chains; wherein each of the heavy chains includes on its surface at least three cysteine residues (third amino acid residues);
(a2-3) TCEP (a first reducing agent) was added, to dissociate interchain disulfide bonds of the Trastuzumab (antibody) and therefore expose first free thiols in cysteine residues (the first amino acid residues and the third amino acid residues);
(a3-3) a first functional unit including a linker residue of -TR-(Y)m1-(PEG)m2-(Z)m3-(AA)m4-(Spacer)m5- and exatecan (a first functional moiety) was added, to link the cysteine residues (the first amino acid residues and the third amino acid residues) and the first functional unit via a covalent bond between the first free thiols in cysteine residues and the TR residue in the first functional unit, wherein the first functional unit has a structure represented by the following formula:

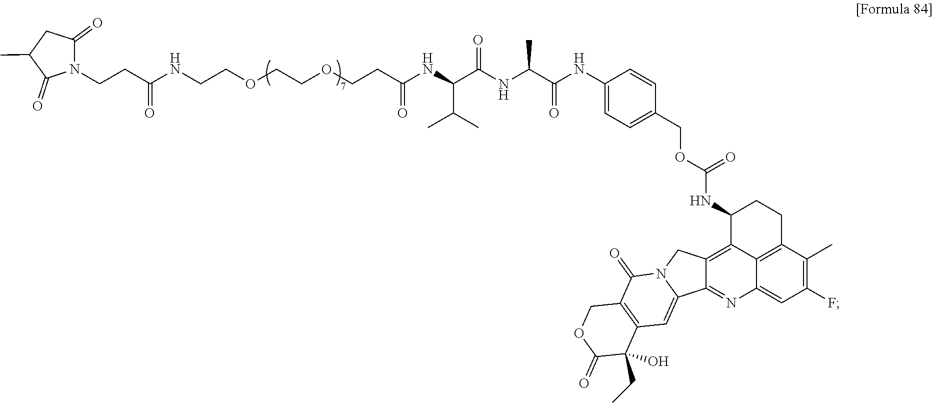

and
wherein, the procedure (B-3) for linking Trastuzumab (antibody) with the second functional unit includes the following steps (b1-3) to (b2-3):
(b1-3) an AAR linker having a structure represented by the formula (AAR)-(PEG)n1-(W)n2-(PEG)n3-(CC1)n4 was added at 4° C., to generate a linker residue including a BL residue which covalently bonded to the lysine residues (the second amino acid residues) via the BL residue, wherein the AAR linker has a structure represented by the following formula:

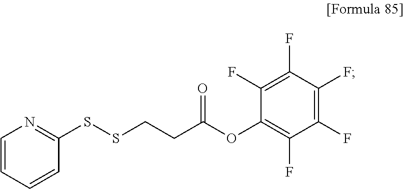 [SMILES: O=C(CCSSc1ccccn1)Oc1c(F)c(F)c(F)c(F)c1F]

the reaction mixture was then placed in the centrifuge filtration tube with membrane at 30 kDa cut-off for washing steps. The washing steps included (1) centrifuge the centrifuge filtration tube containing AAR linker-modified Trastuzumab to remove unreacted AAR linker and (2) wash the retained AAR linker-conjugated Trastuzumab with 1× volume of washing buffer to assure removing unreacted AAR linker; and
(b2-3) a molecule including MMAE (MMAE is the second functional moiety) was added, to link the lysine residues (the second amino acid residues) and the molecules including MMAE (MMAE is the second functional moiety) via the reaction between the linker residue and the molecule including MMAE; wherein the molecule including MMAE has a structure represented by the following formula:

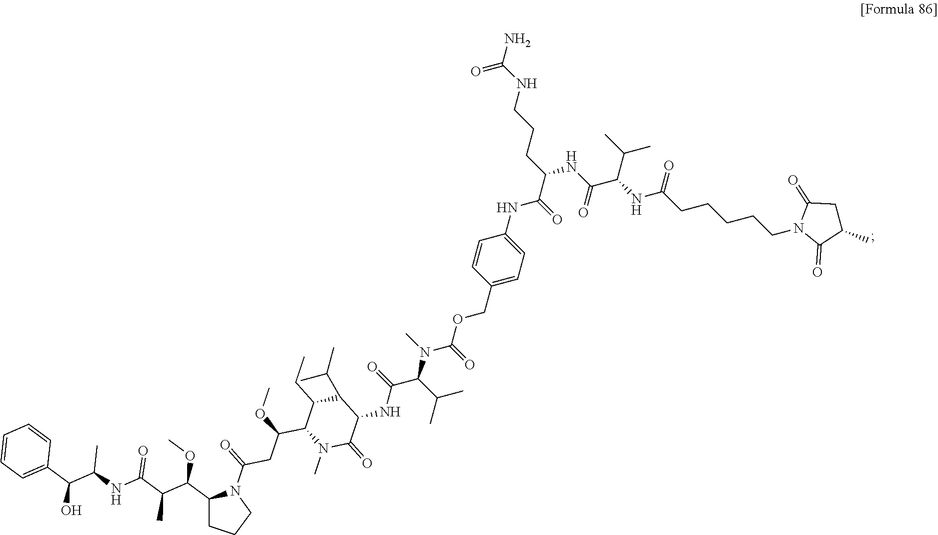

and wherein the step (b2-3) includes substeps.
(b21-3) TCEP (a second reducing agent) was added at room temperature, to reduce a disulfide bond in the linker residue and therefore to expose second free thiols in the linker residue; and
(b22-3) the molecule including MMAE (MMAE is the second functional moiety) was added, to react the second free thiols with the molecule including MMAE, and therefore to link the lysine residues (the second amino acid residues) and the molecules including MMAE (MMAE is the second functional moiety) via the reaction between the second free thiol and the molecule including MMAE.

The reaction mixture was then washed as previously described.

The concentration of retained Trastuzumab-exatecan-vcMMAE at DAR 10 was then quantified by the BCA quantification method.

The conjugation buffer can be selected from the group consisting of a phosphate buffer, an acetate buffer, a Tris buffer, a citrate buffer, a sodium chloride solution, an EDTA buffer, or an amino acid-containing buffer.

For the first functional unit including the linker residue of -TR-(Y)m1-(PEG)m2-(Z)m3-(AA)m4-(Spacer)m5- and exatecan (a first functional moiety) described in this Embodiment 3-1, the TR residue is a maleimide residue (or -(Succinimid-3-yl-N)—) which includes a sulfhydryl-reactive group, Y is a Y molecule residue including an alkyl group having a structure represented by the following formula:

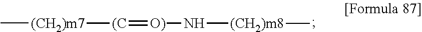

Z residue is a Z molecule residue having a structure represented by the following formula:

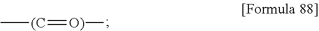

-(AA)m4- is a peptide linkage made by linking a valine residue and an alanine residue; and Spacer residue is a first linker unit connecting to the first functional moiety and having a structure represented by the following formula:

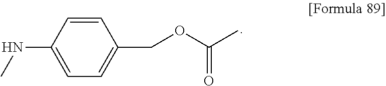

For the AAR linker having the structure represented by the formula (AAR)-(PEG)n1-(W)n2-(PEG)n3-(CC1)n4 described in this Embodiment 3-1, W is a functional linker residue including —C(═O)—(CH₂)—(CH₂)—; CC1 is a reaction moiety of a bioorthogonal pair having a structure represented by the following formula:

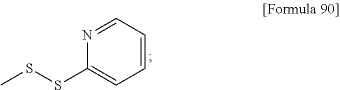

and
AAR has a structure represented by the following formula:

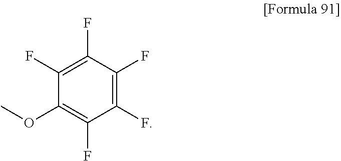

The product in this Embodiment 3-1 includes a second functional unit, wherein the second functional unit includes MMAE (a second functional moiety) and a linker residue (a second linker residue) of -(BL)-(PEG)n1-(W)n2-(PEG)n3-(CC1)n4-(CC2)n4-(PEG)n5-(NH—C(═O)CH₂—O—CH₂)n19-(C(═O))n14-(AA)n6-(Spacer)n7-; wherein
BL residue is a single bond,
W residue is —C(═O)—(CH₂)—(CH₂)—,
—CC1- residue is —S—,
—CC2- residue is (Succinimid-3-yl-N)—R⁴ having a structure represented by the following formula:

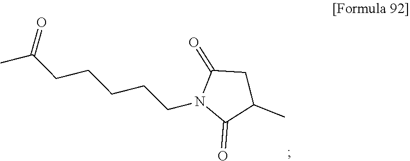 [SMILES: CC(=O)CCCCCN1C(=O)CC(C)C1=O]

-(AA)n6- is a peptide linkage made by linking a valine residue and a citrulline residue; and Spacer residue has a structure represented by the following formula

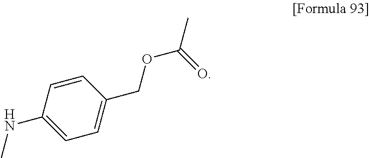

### Embodiment 3-2: Characterization of Trastuzumab-Exatecan-vcMMAE (DAR 10)

RP-HPLC and mass spectrometry were applied for the Trastuzumab-exatecan-vcMMAE (DAR 10) prepared according to the process described in Embodiment 3-1.

There were two groups in this experiment:
Trastuzumab-exatecan (DAR8) was prepared according to the procedure (A) to conjugate 8 numbers of the first functional moieties (exatecan) described in FIG. 10; and
Trastuzumab-exatecan-vcMMAE (DAR10) was prepared according to the procedure (A) to conjugate 8 numbers of the first functional moieties (exatecan) as well as procedure (B) to conjugate 2 numbers of the second functional moiety (MMAE) described in FIG. 10.

For the RP-HPLC analysis, 20 μg of reduced sample was injected into the RP-HPLC Agilent 1260 Infinity II System equipped with reverse phase column (AdvanceBioRP mAb Diphenyl, 4.6 mm×150 mm, 3.5 μm) and the signal was detected at 280 nm absorbance. The corresponding peaks of signals were presented as retention time.

The light chain of Trastuzumab-exatecan (DAR8, the light chain is represented by LC+1D) was at 10.170 minute, and light chain of Trastuzumab-exatecan-vcMMAE (DAR10, the light chain is represented by LC+1D+1V) shifted to about 12.886 minute, whereas the heavy chain showed no shift in Trastuzumab-exatecan-vcMMAE (DAR10).

To further verify the light chain-specific conjugation, liquid chromatography equipped with mass spectrometry was applied. The peaks in the liquid chromatography sequentially injected into the mass spectrometry. The analysis revealed that the characterization and the mass peak of the light chain (represented by LC+1D+1V) and the heavy chain (represented by HC+3D) of Trastuzumab-exatecan-vcMMAE (DAR 10) could be confirmed.

### Embodiment 3-3: Binding Affinity of Trastuzumab-Exatecan-vcMMAE (DAR 10)

Binding affinity experiment was applied for the Trastuzumab-exatecan-vcMMAE (DAR 10) prepared according to the process described in Embodiment 3-1. There were two groups in this experiment:
HER2 mAb was the antibody “Trastuzumab” (DAR0); and
Trastuzumab-exatecan-vcMMAE was prepared according to the procedure (A) to conjugate 8 numbers of the first functional moieties (exatecan) as well as procedure (B) to conjugate 2 numbers of the second functional moiety (MMAE) to Trastuzumab as described in FIG. 10 (DAR10).

A 96-well white plate was coated with His-tagged human HER2 recombinant protein and then blocked with 5% skim milk. The blocked His-tagged human HER2 recombinant protein-coated white plate was then incubated with Trastuzumab or Trastuzumab-exatecan-vcMMAE (DAR10) at different concentrations at 37° C. for 1 hour. The unbound Trastuzumab or Trastuzumab-exatecan-vcMMAE (DAR10) was washed away with PBST. The bound Trastuzumab or Trastuzumab-exatecan-vcMMAE (DAR10) was detected by HRP-conjugated anti-human IgG and the developed color signal was recorded at 450 nm.

The results revealed that there was no significant difference of signal detection between Trastuzumab and Trastuzumab-exatecan-vcMMAE (DAR10) among the concentration range, suggesting the conjugation of 8 numbers of exatecan and 2 numbers of MMAE elicited no impact on the HER2 binding of Trastuzumab. This is an unexpected result. That is, a person skilled in the art would expect that conjugation of 2 numbers of 1.5 kDa linker-payload to a 25 kDa light chain of the antibody should results in steric hindrance to significantly impair antigen-binding affinity. However, the results shows that conjugation of 1 number of exatecan and 1 number of MMAE to each of the light chains and conjugation of 3 numbers of MMAE to each of the heavy chains elicited no impact on the HER2 binding of Trastuzumab, it is an unexpected result.

### Embodiment 3-4: Cytotoxicity of Trastuzumab-Exatecan-vcMMAE (DAR 10)

Cytotoxicity assay was applied for the Trastuzumab-exatecan-vcMMAE (DAR 10) prepared according to the process described in Embodiment 3-1. There were four groups in this experiment:
Trastuzumab was the antibody (DAR0).
Trastuzumab-exatecan (DAR 8) was prepared according to the procedure (A) to conjugate 8 numbers of the first functional moieties (exatecan) to Trastuzumab as described in FIG. 10 (DAR10).
Trastuzumab AD2C was Trastuzumab-exatecan-vcMMAE (DAR 10) prepared according to the procedure (A) to conjugate 8 numbers of the first functional moieties (exatecan) and procedure (B) to conjugate 2 numbers of the second functional moiety (MMAE) to Trastuzumab as described in FIG. 10 (DAR10).
Enhertu was Trastuzumab-deruxtecan conjugation acts as positive control.

One thousand human HER2-positive breast cancer cell line SK-BR-3 or human triple-negative breast cancer MDA-MB-231 cells (HER2-negative) were seeded into each well of a white flat-bottom 96-well plate for an overnight incubation and then treated with Trastuzumab, Trastuzumab-exatecan (DAR8), Trastuzumab-exatecan-vcMMAE (DAR10), or Trastuzumab-deruxtecan at concentration 0.003, 0.002, 0.009, 0.04, 0.20, 1.06, 5.33, 26.67, 133.3 nM for 6 days. Each condition was performed in triplicate. On the harvesting day, the number of viable cells in each well were measured according to the CellTiter-Glo™ 2.0 Assay process.

FIG. 11A and FIG. 11B are cytotoxicity results indicate that the product prepared according to the process described in FIG. 10 exhibit cytotoxicity against human HER2-positive breast cancer cell line SK-BR-3. FIG. 12 is cytotoxicity results indicate that the product prepared according to the process described in FIG. 10 exhibit cytotoxicity against human triple-negative breast cancer cell line MDA-MB-231 (HER2-negative).

The results in FIG. 11A and FIG. 11B revealed that Trastuzumab-exatecan-vcMMAE (DAR10) had better cytotoxicity against SK-BR-3 cells than Trastuzumab (DAR0) and Enhertu (p<0.05), suggesting high-Dar ADCs exhibit higher cytotoxicity against SK-BR-3 cells (HER2-positive breast cancer cells). On the other hand, the results in FIG. 12 revealed that Trastuzumab-exatecan-vcMMAE (DAR10), Trastuzumab (DAR0), and Enhertu had similar cytotoxicity against MDA-MB-231 (HER2-negative breast cancer cell). These results supported that the dual payload types of platform in trastuzumab model harbored promising and better potency than mono payload type or antibody alone as well as FDA approved anti-HER2 ADC Enhertu.

### Embodiment 4: Anti-GPC3 mAb-Exatecan-MMAE (Cysteine and Lysine Based Conjugation, AAR Linker Includes —S— Group)

### Embodiment 4-1: Preparation of Anti-GPC3 mAb-Exatecan-MMAE (DAR 10) Via an AAR Linker Including —S— Group

Please refer to FIG. 13, which illustrates one process of manufacturing a DAR10 Anti-GPC3 mAb-exatecan-MMAE via procedure (A) conjugating 8 numbers of the first functional moieties (exatecan) and procedure (B) conjugating 2 numbers of the second functional moiety (MMAE) to an antibody (Anti-GPC3), wherein the first functional moieties are linked to the cysteine residue on the surface of the light chain and the heavy chain of the antibody via the first linker residue, and the second functional moieties are linked to the lysine residue on the surface of the light chain of the antibody via the second linker residue.

In this embodiment, Anti-GPC3 mAb is a commercially available anti-GPC3 IgG1 antibody and includes heavy chains and kappa light chains, wherein the kappa light chains comprise a lysine residue corresponding to a position 188 (Lys188) or 190 (Lys190).

The methods of manufacturing the DAR10 Anti-GPC3 mAb-exatecan-MMAE in this embodiment includes at least the procedures for linking an Anti-GPC3 mAb (antibody) with (A-4) a first functional unit and (B-4) a second functional unit;
wherein, the procedure (A-4) for linking the Anti-GPC3 mAb (antibody) with the first functional unit includes at least the following steps (a1-4) to (a3-4):
(a1-4) the Anti-GPC3 mAb (commercially available anti-GPC3 antibody) was provided in a conjugation buffer, wherein the Anti-GPC3 mAb includes:
   a first light chain and a second light chain; each of the light chains includes on its surface a cysteine residue (a first amino acid residue) and a lysine residue (Lys188 or Lys 190, a second amino acid residue), and the first light chain is a kappa light chain; and
   heavy chains; wherein each of the heavy chains includes on its surface at least three cysteine residues (third amino acid residues);
(a2-4) TCEP (a first reducing agent) was added, to dissociate interchain disulfide bonds of the Anti-GPC3 mAb (antibody) and therefore expose first free thiols in cysteine residues (the first amino acid residues and the third amino acid residues);
(a3-4) a first functional unit including a linker residue of -TR-(Y)m1-(PEG)m2-(Z)m3-(AA)m4-(Spacer)m5- and exatecan (a first functional moiety) was added, to link the cysteine residues (the first amino acid residues and the third amino acid residues) and the first functional unit via a covalent bond between the first free thiols in cysteine residues and the TR residue in the first functional unit, wherein the first functional unit has a structure represented by the following formula:

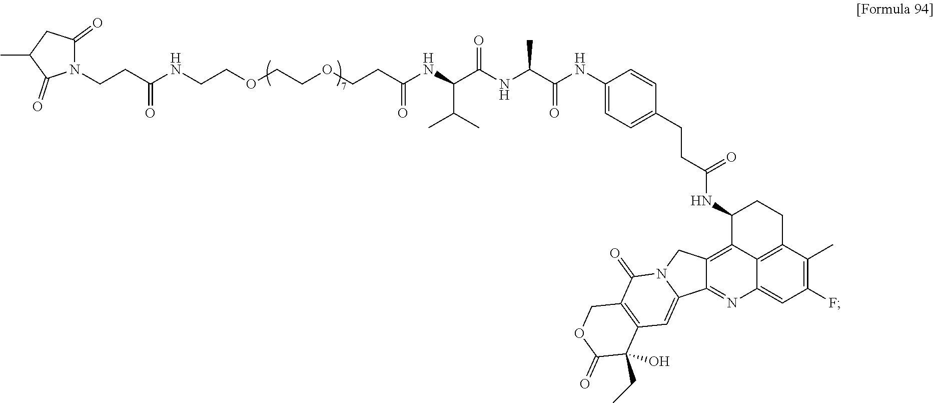

and
wherein, the procedure (B-4) for linking Anti-GPC3 mAb (antibody) with the second functional unit includes the following steps (b1-4) to (b2-4):
(b1-4) an AAR linker having a structure represented by the formula (AAR)-(PEG)n1-(W)n2-(PEG)n3-(CC1)n4 was added at 4° C., to generate a linker residue including a BL residue which covalently bonded to the lysine residues (the second amino acid residues) via the BL residue, wherein the AAR linker has a structure represented by the following formula:

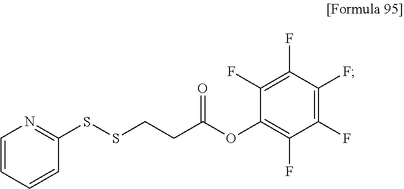 [SMILES: O=C(CCSSc1ccccn1)Oc1c(F)c(F)c(F)c(F)c1F]

the rection mixture was then placed in the centrifuge filtration tube with membrane at 30 kDa cut-off for washing steps. The washing steps included (1) centrifuge the centrifuge filtration tube containing AAR linker-modified Anti-GPC3 mAb to remove unreacted AAR linker and (2) wash the retained AAR linker-conjugated Anti-GPC3 mAb with 1× volume of washing buffer to assure removing unreacted AAR linker; and
(b2-4) a molecule including MMAE (MMAE is the second functional moiety) was added, to link the lysine residues (the second amino acid residues) and the molecules including MMAE (MMAE is the second functional moiety) via the reaction between the linker residue and the molecule including MMAE; wherein the molecule including MMAE has a structure represented by the following formula:

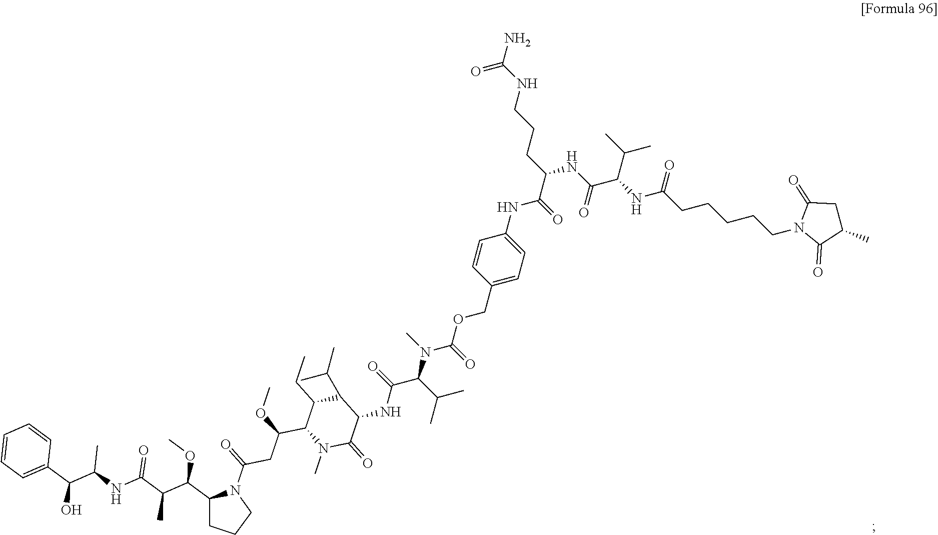

and wherein the step (b2-4) includes substeps:
(b21-4) TCEP (a second reducing agent) was added at room temperature, to reduce a disulfide bond in the linker residue and therefore to expose second free thiols in the linker residue; and
(b22-4) the molecule including MMAE (MMAE is the second functional moiety) was added, to react the second free thiols with the molecule including MMAE, and therefore to link the lysine residues (the second amino acid residues) and the molecules including MMAE (MMAE is the second functional moiety) via the reaction between the second free thiol and the molecule including MMAE.

The reaction mixture was then washed as previously described.

The concentration of retained Anti-GPC3 mAb-exatecan-MMAE at DAR 10 was then quantified by the BCA quantification method.

The conjugation buffer can be selected from the group consisting of a phosphate buffer, an acetate buffer, a Tris buffer, a citrate buffer, a sodium chloride solution, an EDTA buffer, or an amino acid-containing buffer.

For the first functional unit including the linker residue of -TR-(Y)m1-(PEG)m2-(Z)m3-(AA)m4-(Spacer)m5- and exatecan (a first functional moiety) described in this Embodiment 4-1, the TR residue is a maleimide residue (or -(Succinimid-3-yl-N)—) which includes a sulfhydryl-reactive group, Y is an a Y molecule residue including an alkyl group having a structure represented by the following formula:

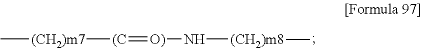

Z residue is an Z molecule residue having a structure represented by the following formula:

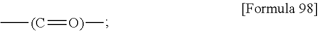

-(AA)m4- is a peptide linkage made by linking a valine residue and an alanine residue; and Spacer residue is a first linker unit connecting to the first functional moiety and having a structure represented by the following formula:

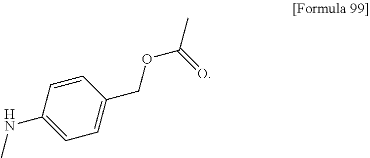

For the AAR linker having the structure represented by the formula (AAR)-(PEG)n1-(W)n2-(PEG)n3-(CC1)n4 described in this Embodiment 4-1, W is a functional linker residue including —C(═O)—(CH₂)—(CH₂)—; CC1 is a reaction moiety of a bioorthogonal pair having a structure represented by the following formula:

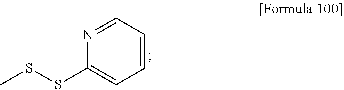

and
AAR has a structure represented by the following formula:

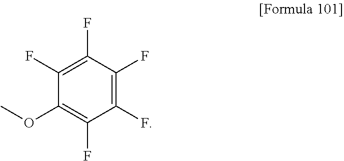

The product in this Embodiment 4-1 includes a second functional unit, wherein the second functional unit includes MMAE (a second functional moiety) and a linker residue (a second linker residue) of -(BL)-(PEG)n1-(W)n2-(PEG)n3-(CC1)n4-(CC2)n4-(PEG)n5-(NH—C(═O)CH₂—O—CH₂)n19-(C(═O))n14-(AA)n6-(Spacer)n7-; wherein
BL residue is a single bond,
W residue is —C(═O)—(CH₂)—(CH₂)—,
—CC1- residue is —S—,
—CC2- residue is (Succinimid-3-yl-N)—R⁴ having a structure represented by the following formula:

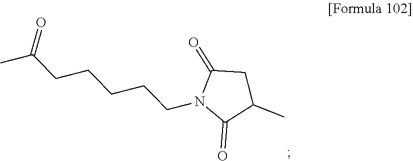 [SMILES: CC(=O)CCCCCN1C(=O)CC(C)C1=O]

-(AA)n6- is a peptide linkage made by linking a valine residue and a citrulline residue; and Spacer residue has a structure represented by the following formula

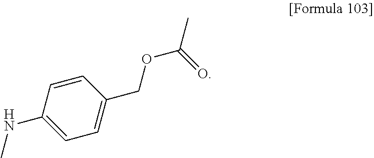

### Embodiment 5: Anti-GPC3 mAb-Exatecan-MMAE (Cysteine and Lysine Based Conjugation, AAR Linker Includes Azide Group)

### Embodiment 5-1: Preparation of Anti-GPC3 mAb-Exatecan-MMAE (DAR 10) Via an AAR Linker Including Azide Group and PFP

Please refer to FIG. 14, which illustrates another process of manufacturing a DAR10 Anti-GPC3 mAb-exatecan-MMAE via procedure (A) conjugating 8 numbers of the first functional moieties (exatecan) and procedure (B) conjugating 2 numbers of the second functional moiety (MMAE) to an antibody (Anti-GPC3 antibody), wherein the first functional moieties are linked to the cysteine residue on the surface of the light chain and the heavy chain of the antibody via the first linker residue, and the second functional moieties are linked to the lysine residue on the surface of the light chain of the antibody via the second linker residue.

In this embodiment, Anti-GPC3 mAb is a commercially available anti-GPC3 IgG1 antibody and includes heavy chains and kappa light chains, wherein the kappa light chains comprise a lysine residue corresponding to a position 188 (Lys188) or 190 (Lys190).

The methods of manufacturing the DAR10 Anti-GPC3 mAb-exatecan-MMAE in this embodiment includes at least the procedures for linking an Anti-GPC3 mAb (antibody) with (A-5) a first functional unit and (B-5) a second functional unit;
wherein, the procedure (A-5) for linking the Anti-GPC3 mAb (antibody) with the first functional unit includes at least the following steps (a1-5) to (a3-5):
(a1-5) the Anti-GPC3 mAb (commercially available anti-GPC3 antibody) was provided in a conjugation buffer, wherein the Anti-GPC3 mAb includes:
   a first light chain and a second light chain; each of the light chains includes on its surface a cysteine residue (a first amino acid residue) and a lysine residue (Lys188 or Lys 190, a second amino acid residue), and the first light chain is a kappa light chain; and
   heavy chains; wherein each of the heavy chains includes on its surface at least three cysteine residues (third amino acid residues);
(a2-5) TCEP (a first reducing agent) was added, to dissociate interchain disulfide bonds of the Anti-GPC3 mAb (antibody) and therefore expose first free thiols in cysteine residues (the first amino acid residues and the third amino acid residues);
(a3-5) a first functional unit including a linker residue of -TR-(Y)m1-(PEG)m2-(Z)m3-(AA)m4-(Spacer)m5- and exatecan (a first functional moiety) was added, to link the cysteine residues (the first amino acid residues and the third amino acid residues) and the first functional unit via a covalent bond between the first free thiols in cysteine residues and the TR residue in the first functional unit, wherein the first functional unit has a structure represented by the following formula:

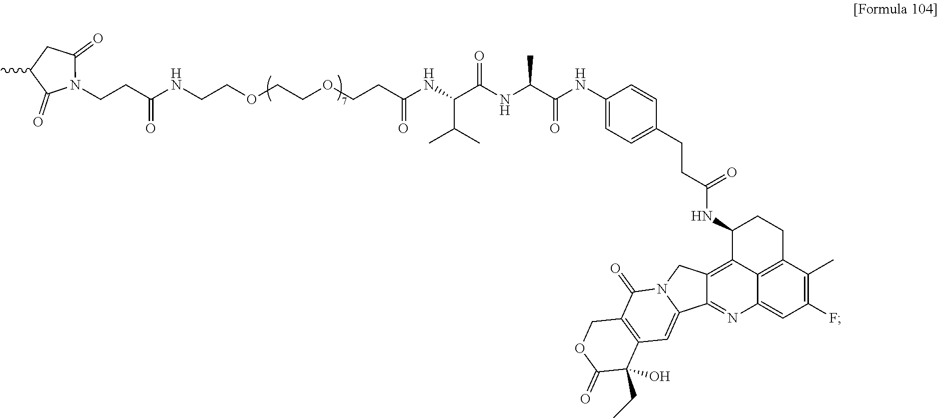

and
wherein, the procedure (B-5) for linking Anti-GPC3 mAb (antibody) with the second functional unit includes the following steps (b1-5) to (b2-5):
(b1-5) an AAR linker having a structure represented by the formula (AAR)-(PEG)n1-(W)n2-(PEG)n3-(CC1)n4 was added at 4° C., to generate a linker residue including a BL residue which covalently bonded to the lysine residues (the second amino acid residues) via the BL residue, wherein the AAR linker has a structure represented by the following formula:

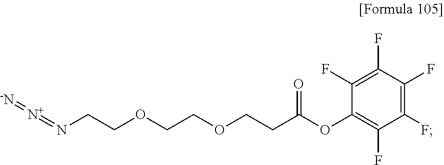

the rection mixture was then placed in the centrifuge filtration tube with membrane at 30 kDa cut-off for washing steps. The washing steps included (1) centrifuge the centrifuge filtration tube containing AAR linker-modified Anti-GPC3 mAb to remove unreacted AAR linker and (2) wash the retained AAR linker-conjugated Anti-GPC3 mAb with 1× volume of washing buffer to assure removing unreacted AAR linker; and
(b2-5) a molecule including MMAE (MMAE is the second functional moiety) was added, to link the lysine residues (the second amino acid residues) and the molecules including MMAE (MMAE is the second functional moiety) via the reaction between the linker residue and the molecule including MMAE; wherein the molecule including MMAE has a structure represented by the following formula:

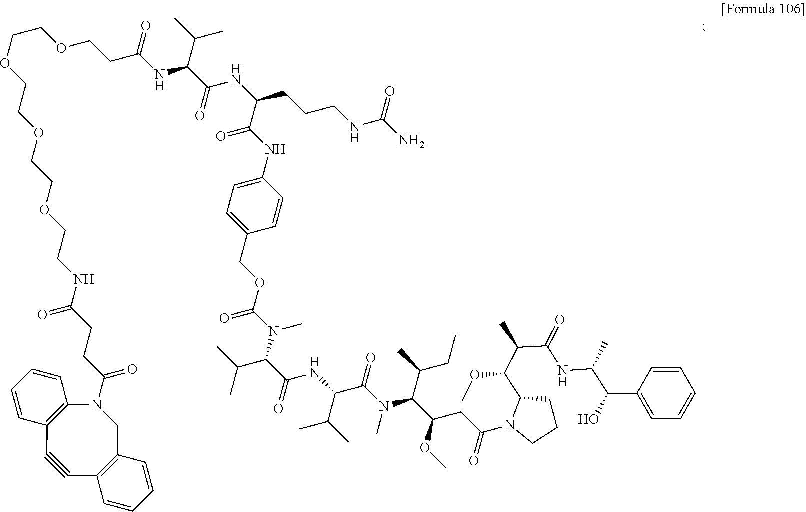 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCOCCOCCOCCNC(=O)CCC(=O)N2Cc3ccccc3C#Cc3ccccc32)C(C)C)cc1)C(C)C]

The reaction mixture was then washed as previously described.

The concentration of retained Anti-GPC3 mAb-exatecan-MMAE at DAR 10 was then quantified by the BCA quantification method.

The conjugation buffer can be selected from the group consisting of a phosphate buffer, an acetate buffer, a Tris buffer, a citrate buffer, a sodium chloride solution, an EDTA buffer, or an amino acid-containing buffer.

For the first functional unit including the linker residue of -TR-(Y)m1-(PEG)m2-(Z)m3-(AA)m4-(Spacer)m5- and exatecan (a first functional moiety) described in this Embodiment 5-1, the TR residue is a maleimide residue (or -(Succinimid-3-yl-N)—) which includes a sulfhydryl-reactive group, Y is a Y molecule residue including an alkyl group having a structure represented by the following formula:

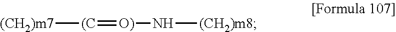

Z residue is a Z molecule residue having a structure represented by the following formula:

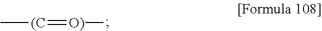

-(AA)m4- is a peptide linkage made by linking a valine residue and an alanine residue; and Spacer residue is a first linker unit connecting to the first functional moiety and having a structure represented by the following formula:

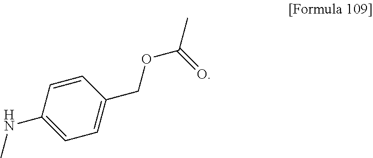

For the AAR linker having the structure represented by the formula (AAR)-(PEG)n1-(W)n2-(PEG)n3-(CC1)n4 described in this Embodiment 5-1, W is a functional linker residue including —C(═O)—(CH₂)—(CH₂)—; CC1 is a reaction moiety of a bioorthogonal pair having a structure represented by the following formula:

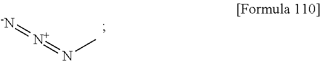

and
AAR has a structure represented by the following formula:

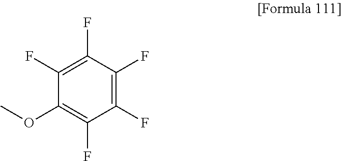

The product in this Embodiment 5-1 includes a second functional unit, wherein the second functional unit in this embodiment includes MMAE (a second functional moiety) and a linker residue (a second linker residue) of -(BL)-(PEG)n1-(W)n2-(PEG)n3-(CC1)n4-(CC2)n4-(PEG)n5-(NH—C(═O)CH₂—O—CH₂)n19-(C(═O))n14-(AA)n6-(Spacer)n7-; wherein
BL residue in this embodiment is a single bond,
W residue in this embodiment is —C(═O)—(CH₂)—(CH₂)—,
—CC1- residue in this embodiment is N₃—R⁵ and R⁵ in this embodiment is a single bond,
—CC2- residue in this embodiment is DBCO-R⁶ having a structure represented by the following formula:

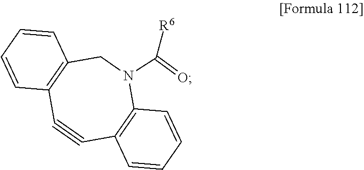

wherein R⁶ in this embodiment is —(CH₂)n11-C(═O)—(NH)n12-(CH₂)n13-, and n11, n12 and n13 are independently integers from 0 to 30;
-(AA)n6- is a peptide linkage made by linking a valine residue and a citrulline residue; and Spacer residue has a structure represented by the following formula

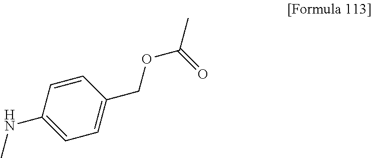

### Embodiment 5-2: Characterization of Anti-GPC3 mAb-Exatecan-MMAE (DAR 10)

RP-HPLC and mass spectrometry were applied for the Anti-GPC3 mAb-exatecan-MMAE (DAR 10) prepared according to the process described in Embodiment 5-1.

There were four groups in this experiment:
Anti-GPC3 mAb was the antibody (DAR0).
Anti-GPC3 mAb-PFP was the antibody that reacted with PFP-based linker (DAR0).
Anti-GPC3 mAb-vcMMAE was prepared according to the procedure (A) to conjugate 2 numbers of the second functional moieties (MMAE) as described in FIG. 14 (DAR2).
Anti-GPC3 mAb-exatecan-vcMMAE was Anti-GPC3 mAb-exatecan-MMAE prepared according to the procedure (A) to conjugate 8 numbers of the first functional moieties (exatecan) and procedure (B) to conjugate 2 numbers of the second functional moiety (MMAE) as described in FIG. 14 (DAR10).

For the RP-HPLC analysis, 20 μg of reduced sample was injected into the RP-HPLC Agilent 1260 Infinity II System equipped with reverse phase column (AdvanceBioRP mAb Diphenyl, 4.6 mm×150 mm, 3.5 μm) and the signal was detected at 280 nm absorbance. The corresponding peaks of signals were presented as retention time.

Please refer to FIG. 15, which is the Reverse phase HPLC (RP-HPLC) results of the product prepared according to the process described in FIG. 14. The light chain of Anti-GPC3 mAb-MMAE (DAR2, the light chain is represented by LC+1V) was at 9.560 minute, and light chain of Anti-GPC3 mAb-exatecan-MMAE (DAR10, the light chain is represented by LC+1D+1V) shifted to about 10.661 minute.

To further verify the light chain-specific conjugation, liquid chromatography equipped with mass spectrometry was applied. The peaks in the liquid chromatography sequentially injected into the mass spectrometry. The analysis revealed that the characterization and the mass peak of the light chain (represented by LC+1D+1V) and the heavy chain (represented by HC+3D) of Anti-GPC3 mAb-exatecan-MMAE (DAR 10) could be confirmed.

### Embodiment 5-3: Binding Affinity and Internalization of Anti-GPC3 mAb-Exatecan-MMAE (DAR 10)

Binding affinity experiment and internalization experiment were applied for the Anti-GPC3 mAb-exatecan-MMAE (DAR 10) prepared according to the process described in Embodiment 5-1. There were two or three groups in these experiments:
α GPC3 was the anti-GPC3 monoclonal antibody (DAR0).
α GPC3 AD2C was the Anti-GPC3 mAb-exatecan-MMAE prepared according to the procedure (A) to conjugate 8 numbers of the first functional moieties (exatecan) and procedure (B) to conjugate 2 numbers of the second functional moiety (MMAE) described in FIG. 14 (DAR10).
Human IgG was an IgG antibody that lacks specificity binding capacity to a cell-surface target protein GPC3 and therefore cannot trigger endocytosis to internalize the antibody into the cell, acts as negative control.

For the binding affinity experiment, a 96-well white plate was coated with His-tagged human GPC3 recombinant protein and then blocked with 5% skim milk. The blocked His-tagged human GPC3 recombinant protein-coated white plate was then incubated with anti-GPC3 monoclonal antibody or Anti-GPC3 mAb-exatecan-MMAE (DAR10) at different concentrations at 37° C. for 1 hour. The unbound anti-GPC3 monoclonal antibody or Anti-GPC3 mAb-exatecan-MMAE was washed away with PBST. The bound anti-GPC3 monoclonal antibody and Anti-GPC3 mAb-exatecan-MMAE was detected by HRP-conjugated anti-human IgG and the developed color signal was recorded at 450 nm.

For the internalization experiment, commercially available internalization assay and human liver cancer cells HepG2 were used.

Please refer to FIG. 16, which is the binding affinity results of the product prepared according to the process described in FIG. 14. The results revealed that there was no significant difference of signal detection between anti-GPC3 monoclonal antibody and Anti-GPC3 mAb-exatecan-MMAE (DAR10) among the concentration range, suggesting the conjugation of 8 numbers of exatecan and 2 numbers of MMAE elicited no impact on the GPC3 binding of Anti-GPC3 mAb. This is an unexpected result. That is, a person skilled in the art would expect that conjugation of 2 numbers of 1.5 kDa linker-payload to a 25 kDa light chain of the antibody should results in steric hindrance to significantly impair antigen-binding affinity. However, FIG. 16 shows that conjugation of 1 numbers of exatecan and 1 numbers of MMAE to each of the light chains and conjugation of 3 numbers of exatecan to each of the heavy chains elicited no impact on the GPC3 binding of Anti-GPC3 mAb, it is an unexpected result.

Moreover, based on the binding affinity result shown in FIG. 16, the EC50 of Anti-GPC3 mAb-exatecan-MMAE (DAR10) is 0.113 nM, which is less than 2 times of EC50 of Anti-GPC3 mAb (0.113 is less than 2×0.074).

Please refer to FIG. 17A and FIG. 17B, which are antibody internalization results. The results revealed that the antibody internalization for human liver cancer cell line HepG2 is unchanged after the procedure (A) to conjugate 8 numbers of the first functional moieties (exatecan) and procedure (B) to conjugate 2 numbers of the second functional moiety (MMAE) to the antibody as described in FIG. 14.

### Embodiment 5-4: Target Cell Binding Capacity and Cytotoxicity of Anti-GPC3 mAb-Exatecan-MMAE (DAR 10)

Target cell binding capacity experiment and cytotoxicity assay were applied for the Anti-GPC3 mAb-exatecan-MMAE (DAR 10) prepared according to the process described in Embodiment 5-1. There were two or three groups in these experiments:
α GPC3 was the anti-GPC3 monoclonal antibody (DAR0).
α GPC3 AD2C was the Anti-GPC3 mAb-exatecan-MMAE prepared according to the procedure (A) to conjugate 8 numbers of the first functional moieties (exatecan) and procedure (B) to conjugate 2 numbers of the second functional moiety (MMAE) as described in FIG. 14 (DAR10).
α GPC3-payload 1 was Anti-GPC3 mAb-exatecan prepared according to the procedure (A) to conjugate 8 numbers of the first functional moieties (exatecan) as described in FIG. 14 (DAR8).
α GPC3-payload 2 was Anti-GPC3 mAb-MMAE prepared according to the procedure (B) to conjugate 2 numbers of the second functional moieties (MMAE) as described in FIG. 14 (DAR2).

For the target cell binding capacity experiment, human liver cancer cells (cell line HepG2, Hep3B, JHH-5, JHH-7, or Huh-7) were treated with anti-GPC3 monoclonal antibody or Anti-GPC3 mAb-exatecan-MMAE (DAR10). The unbound anti-GPC3 monoclonal antibody or Anti-GPC3 mAb-exatecan-MMAE was washed away with PBST. The cells bound by anti-GPC3 monoclonal antibody or Anti-GPC3 mAb-exatecan-MMAE were mixed with fluorescently labeled anti-human IgG and analyzed using a flow cytometry.

For the cytotoxicity experiment, one thousand human liver cancer cells (cell line HepG2, Hep3B, JHH-5, JHH-7, or Huh-7) were seeded into each well of a white flat-bottom 96-well plate for an overnight incubation and then treated with anti-GPC3 monoclonal antibody, Anti-GPC3 mAb-MMAE (DAR2), Anti-GPC3 mAb-exatecan (DAR8), or Anti-GPC3 mAb-exatecan-MMAE (DAR10) at concentration 0.01, 0.05, 0.2, 1, 2, 5, 20, 50, 100, 500 nM for 6 days. Each condition was performed in triplicate. On the harvesting day, the number of viable cells in each well were measured according to the CellTiter-Glo™ 2.0 Assay process.

Please refer to FIG. 18, which is the results showing the binding capacity of the product prepared according to the process described in FIG. 14 to the cell-surface protein GPC3. The results indicate that the binding capacity of the anti-GPC3 monoclonal antibody to the cell-surface protein GPC3 on human liver cancer cell line HepG2, Hep3B, JHH-5, JHH-7, and Huh-7 is unchanged after the procedure (A) to conjugate 8 numbers of the first functional moieties (exatecan) and procedure (B) to conjugate 2 numbers of the second functional moiety (MMAE) to the antibody as described in FIG. 14.

Please refer FIG. 19. The cytotoxicity results indicate that the Anti-GPC3 mAb-exatecan-MMAE (DAR10) prepared according to the process described in FIG. 14 had significantly enhanced cytotoxicity against human liver cancer cell line HepG2, Hep3B, JHH-5, JHH-7, and Huh-7 compared to anti-GPC3 mAb. These results supported that the dual payload types platform in anti-GPC3 mAb model harbored promising and better potency than antibody alone.

Please refer to FIG. 20A and FIG. 20B. The cytotoxicity results indicate that compared with the anti-GPC3 mAb, the Anti-GPC3 mAb-exatecan-MMAE (DAR10) prepared according to the process described in FIG. 14 exhibit better cytotoxicity against human liver cancer cell line HepG2 and Huh-7, suggesting high-DAR ADCs exhibits higher cytotoxicity.

### Embodiment 5-5: In Vivo Antitumor Activity of Anti-GPC3 mAb-Exatecan-MMAE (DAR 10) in the Mouse Model with Solid Tumor

Mouse with solid tumor was used in this embodiment to confirm the in vivo antitumor activity of Anti-GPC3 mAb-exatecan-MMAE (DAR 10) prepared according to the process described in Embodiment 5-1. There were three groups in these experiments: Vehicle group: mice were intravenously injected with normal saline.
α GPC3 group: mice were intravenously injected with the anti-GPC3 monoclonal antibody (DAR0).
α GPC3 AD2C group: mice were intravenously injected with the product prepared according to the procedure (A) conjugating 8 numbers of the first functional moieties (exatecan) and procedure (B) conjugating 2 numbers of the second functional moiety (MMAE) described in FIG. 14 (DAR10).

5×10⁶ luciferase-expressing target cells (human liver cancer cell line HepG2) were subcutaneously injected into each of the 15 mice (Jackson Laboratory). The mice were divided into 3 groups and intravenously injected with normal saline, anti-GPC3 monoclonal antibody (DAR0), or anti-GPC3 mAb-exatecan-MMAE when tumor size reached 150-200 mm3 and set the treatment date as Day 1. Tumor size was measured by caliper and the tumor volume was calculated using the formula 0.52×l×w×w on Day 1, 4, 8, 11, 15, 18, 22, 25, 29 and 32. l and w stand for length and width of the tumor.

Please refer to FIG. 21A and FIG. 21B. FIG. 21A shows the tumor volume in mice intravenously injected with anti-GPC3 mAb-exatecan-MMAE prepared according to the process described in FIG. 14; and FIG. 21B shows the Body weight of the mice intravenously injected with anti-GPC3 mAb-exatecan-MMAE prepared according to the process described in FIG. 14.

FIG. 21A shows that as compared with the anti-GPC3 mAb (DAR0), the Anti-GPC3 mAb-exatecan-MMAE (DAR10) prepared according to the process described in FIG. 14 exhibits significant higher antitumor activity in the mouse model with solid tumor, suggesting high-DAR ADCs exhibit higher antitumor activity.

### Embodiment 6: Anti-GPC3 mAb-Exatecan-MMAE (Cysteine and Tyrosine Based Conjugation)

### Embodiment 6-1: Preparation of Anti-GPC3 mAb-Exatecan-MMAE (DAR 10) Via an AAR Linker Including Azide Group and PTDA

Please refer to FIG. 22, which illustrates the process of manufacturing a DAR10 Anti-GPC3 mAb-exatecan-MMAE via procedure (A) conjugating 8 numbers of the first functional moieties (exatecan) and procedure (B) conjugating 2 numbers of the second functional moiety (MMAE) to an antibody (Anti-GPC3 antibody), wherein the first functional moieties are linked to the cysteine residue on the surface of the light chain and the heavy chain of the antibody via the first linker residue, and the second functional moieties are linked to the tyrosine residue on the surface of the light chain of the antibody via the second linker residue.

In this embodiment, Anti-GPC3 mAb is a commercially available anti-GPC3 IgG1 antibody and includes heavy chains and kappa light chains, wherein the kappa light chains comprise a lysine residue corresponding to a position 188 (Lys188) or 190 (Lys190).

The methods of manufacturing the DAR10 Anti-GPC3 mAb-exatecan-MMAE includes at least the procedures for linking an Anti-GPC3 mAb (antibody) with (A-6) a first functional unit and (B-6) a second functional unit;
wherein, the procedure (A-6) for linking the Anti-GPC3 mAb (antibody) with the first functional unit includes at least the following steps (a1-6) to (a3-6):
(a1-6) the Anti-GPC3 mAb (commercially available anti-GPC3 antibody) was provided in a conjugation buffer, wherein the Anti-GPC3 mAb includes:
   a first light chain and a second light chain; each of the light chains includes on its surface a cysteine residue (a first amino acid residue) and a lysine residue (Lys188 or Lys 190, a second amino acid residue), and the first light chain is a kappa light chain; and
   heavy chains; wherein each of the heavy chains includes on its surface at least three cysteine residues (third amino acid residues);
(a2-6) TCEP (a first reducing agent) was added, to dissociate interchain disulfide bonds of the Anti-GPC3 mAb (antibody) and therefore expose first free thiols in cysteine residues (the first amino acid residues and the third amino acid residues);
(a3-6) a first functional unit including a linker residue of -TR-(Y)m1-(PEG)m2-(Z)m3-(AA)m4-(Spacer)m5- and exatecan (a first functional moiety) was added, to link the cysteine residues (the first amino acid residues and the third amino acid residues) and the first functional unit via a covalent bond between the first free thiols in cysteine residues and the TR residue in the first functional unit, wherein the first functional unit has a structure represented by the following formula:

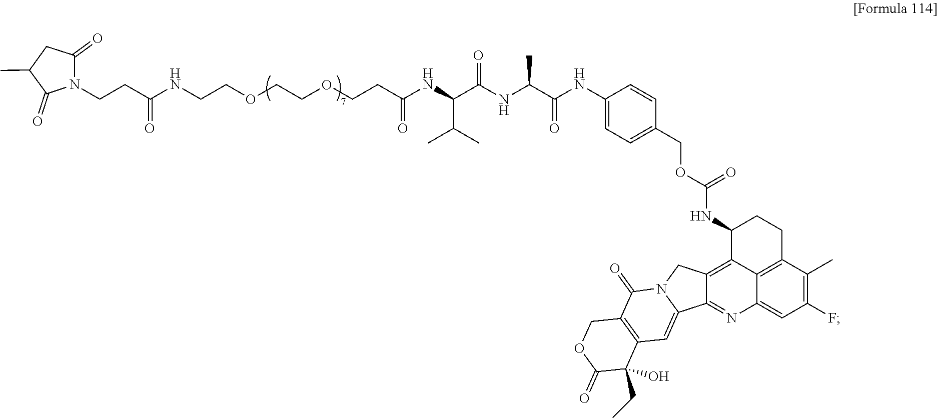

and
wherein, the procedure (B-6) for linking Anti-GPC3 mAb (antibody) with the second functional unit includes the following steps (b1-6) to (b2-6):
(b1-6) an AAR linker having a structure represented by the formula (AAR)-(PEG)n1-(W)n2-(PEG)n3-(CC1)n4 was added at 4° C., to generate a linker residue including a BL residue which covalently bonded to the tyrosine residues (the second amino acid residues) via the BL residue, wherein the AAR linker has a structure represented by the following formula:

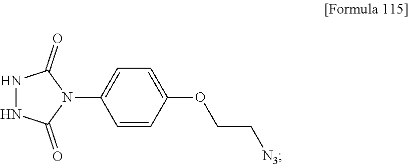 [SMILES: [N-]=[N+]=NCCOc1ccc(-n2c(=O)[nH][nH]c2=O)cc1]

the rection mixture was then placed in the centrifuge filtration tube with membrane at 30 kDa cut-off for washing steps. The washing steps included (1) centrifuge the centrifuge filtration tube containing AAR linker-modified Anti-GPC3 mAb to remove unreacted AAR linker and (2) wash the retained AAR linker-conjugated Anti-GPC3 mAb with 1× volume of washing buffer to ensure removing unreacted AAR linker; and
(b2-6) a molecule including MMAE (i.e., the second functional moiety) was added, to link the tyrosine residues (the second amino acid residues) and the molecules including MMAE via the reaction between the linker residue and the molecule including MMAE; wherein the molecule including MMAE has a structure represented by the following formula:

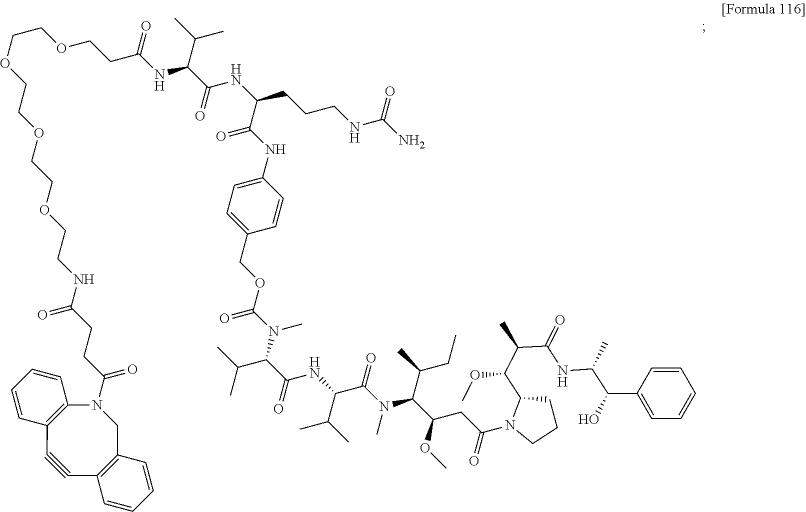 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCOCCOCCOCCNC(=O)CCC(=O)N2Cc3ccccc3C#Cc3ccccc32)C(C)C)cc1)C(C)C]

The reaction mixture was then washed as previously described.

The concentration of retained Anti-GPC3 mAb-exatecan-MMAE at DAR 10 was then quantified by the BCA quantification method.

The conjugation buffer can be selected from the group consisting of a phosphate buffer, an acetate buffer, a Tris buffer, a citrate buffer, a sodium chloride solution, an EDTA buffer, or an amino acid-containing buffer.

For the first functional unit including the linker residue of -TR-(Y)m1-(PEG)m2-(Z)m3-(AA)m4-(Spacer)m5- and exatecan (a first functional moiety) described in this Embodiment 6-1, the TR residue is a maleimide residue (or -(Succinimid-3-yl-N)—) which includes a sulfhydryl-reactive group, Y is an a Y molecule residue including an alkyl group having a structure represented by the following formula.

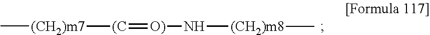

Z residue is a Z molecule residue having a structure represented by the following formula.

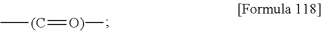

-(AA)m4- is a peptide linkage made by linking a valine residue and an alanine residue; and Spacer residue has a structure represented by the following formula:

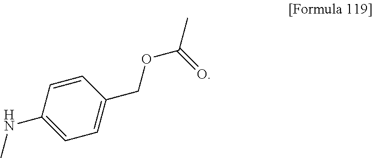

For the AAR linker having the structure represented by the formula (AAR)-(PEG)n1-(W)n2-(PEG)n3-(CC1)n4 described in this Embodiment 6-1, n2 is 0; CC1 is a reaction moiety of a bioorthogonal pair having a structure represented by the following formula:

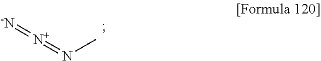

and
AAR has a structure represented by the following formula:

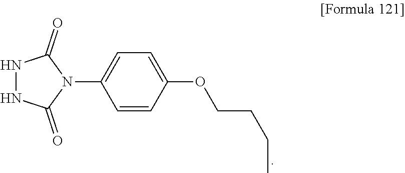 [SMILES: CCCCOc1ccc(-n2c(=O)[nH][nH]c2=O)cc1]

The product in this Embodiment 6-1 includes a second functional unit, wherein the second functional unit in this embodiment includes MMAE (a second functional moiety) and a linker residue (a second linker residue) of -(BL)-(PEG)n1-(W)n2-(PEG)n3—(CC1)n4-(CC2)n4-(PEG)n5-(NH—C(═O)CH₂—O—CH₂)n19-(C(═O))n14-(AA)n6-(Spacer)n7-; wherein
BL residue in this embodiment includes a structure represented by the following formula:

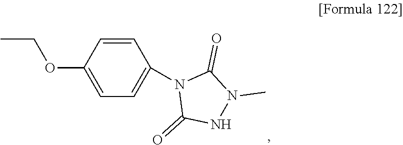 [SMILES: CCOc1ccc(-n2c(=O)[nH]n(C)c2=O)cc1]

n2 is 0,
—CC1- residue in this embodiment is N₃—R⁵ and R⁵ in this embodiment is a single bond,
—CC2- residue in this embodiment is DBCO-R⁶ having a structure represented by the following formula:

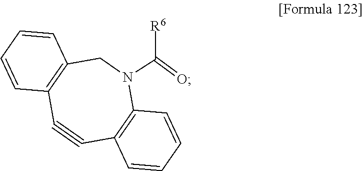

wherein R⁶ in this embodiment is —(CH₂)n11-C(═O)—(NH)n12-(CH₂)n13-, and n11, n12 and n13 are independently integers from 0 to 30;
-(AA)n6- is a peptide linkage made by linking a valine residue and a citrulline residue; and

Spacer residue has a structure represented by the following formula

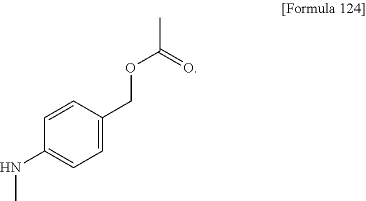

### Embodiment 6-2: Characterization of Anti-GPC3 mAb-Exatecan-MMAE Prepared According to the Procedure as Described in FIG. 22 (DAR10)

RP-HPLC and mass spectrometry were applied for the Anti-GPC3 mAb-exatecan-MMAE (DAR 10) prepared according to the process described in Embodiment 6-1, and the characterization of Anti-GPC3 mAb-exatecan-MMAE (DAR 10) (DAR 10) could be confirmed.

### Embodiment 6-3: Binding Affinity of Anti-GPC3 mAb-Exatecan-MMAE Prepared According to the Procedure as Described in FIG. 22 (DAR10)

The binding affinity experiment was applied for the Anti-GPC3 mAb-exatecan-MMAE (DAR 10) prepared according to the process described in Embodiment 6-1. There were two groups in this experiment:
YP7 mAb was the Anti-GPC3 mAb (anti-GPC3 monoclonal antibody, DAR0).
YP7 mAb-PTDA/MMAE-EXd was the Anti-GPC3 mAb-exatecan-MMAE prepared according to the procedure (A) conjugating 8 numbers of the first functional moieties (exatecan) and procedure (B) conjugating 2 numbers of the second functional moiety (MMAE) as described in FIG. 22 (DAR10).

A 96-well white plate was coated with His-tagged human GPC3 recombinant protein and then blocked with 5% skim milk. The blocked His-tagged human GPC3 recombinant protein-coated white plate was then incubated with Anti-GPC3 mAb, or Anti-GPC3 mAb-exatecan-MMAE (DAR10) at different concentrations at 37° C. for 1 hour. The unbound Anti-GPC3 mAb, or Anti-GPC3 mAb-exatecan-MMAE (DAR10) was washed away with PBST. The bound Anti-GPC3 mAb, or Anti-GPC3 mAb-exatecan-MMAE (DAR10) was detected by HRP-conjugated anti-human IgG and the developed color signal was recorded at 450 nm.

Please refer to FIG. 23, which depicts the binding affinity results of the product prepared according to the process described in FIG. 22. The results revealed that the EC50 of Anti-GPC3 mAb-exatecan-MMAE (DAR10) is less than 2 times of EC50 of Anti-GPC3 mAb.

### Embodiment 6-4: Cytotoxicity of Anti-GPC3 mAb-Exatecan-MMAE Prepared According to the Procedure as Described in FIG. 22 (DAR10)

Cytotoxicity assay was applied for the Anti-GPC3 mAb-exatecan-MMAE (DAR 10) prepared according to the process described in Embodiment 6-1. There were two groups in this experiment:
YP7 mAb was the Anti-GPC3 mAb (anti-GPC3 monoclonal antibody, DAR0).
YP7 mAb-PTDA/MMAE-EXd was the Anti-GPC3 mAb-exatecan-MMAE prepared according to the procedure (A) conjugating 8 numbers of the first functional moieties (exatecan) and procedure (B) conjugating 2 numbers of the second functional moiety (MMAE) as described in FIG. 22 (DAR10).

One thousand human liver cancer cells (cell line JHH-5 or HepG2) were seeded into each well of a white flat-bottom 96-well plate for an overnight incubation and then treated with anti-GPC3 monoclonal antibody or Anti-GPC3 mAb-exatecan-MMAE (DAR10) at concentration 0.003, 0.002, 0.009, 0.04, 0.20, 1.06, 5.33, 26.67, 133.3 nM for 6 days. Each condition was performed in triplicate. On the harvesting day, the number of viable cells in each well were measured according to the CellTiter-Glo™ 2.0 Assay process.

Please refer to FIG. 24A and FIG. 24B, which are the cytotoxicity results indicate that the product prepared according to the process described in FIG. 22 exhibits cytotoxicity against human liver cancer cell line JHH-5 (FIG. 24A) and HepG2 (FIG. 24B). The cytotoxicity results indicate that compared with the anti-GPC3 mAb, the Anti-GPC3 mAb-exatecan-MMAE (DAR10) prepared according to the process described in FIG. 22 exhibit better cytotoxicity against human liver cancer cell line JHH-5 and HepG2, suggesting high-DAR ADCs exhibit higher cytotoxicity.

### Embodiment 7: Effect of -(PEG)n- on the Specificity of PFP-(PEG)n-N3 Linker Mediated DAR10 Antibody Drug Conjugation

PFP-PEG2-azide and PFP-PEG5-azide were used in this embodiment.

In this embodiment, Anti-GPC3 mAb is a commercially available anti-GPC3 IgG1 antibody and includes heavy chains and kappa light chains, wherein the kappa light chains comprise a lysine residue corresponding to a position 188 (Lys188) or 190 (Lys190).

The method of manufacturing the PFP-(PEG)n-N3 linker-conjugated Anti-GPC3 mAb in this embodiment includes the following steps:
(a1-5) Anti-GPC3 mAb (commercially available anti-GPC3 antibody) was provided in a conjugation buffer, wherein the Anti-GPC3 mAb includes:
   a first light chain and a second light chain; each of the light chains includes on its surface a cysteine residue (a first amino acid residue) and a lysine residue (Lys188 or Lys 190, a second amino acid residue), and the first light chain is a kappa light chain; and
   heavy chains; wherein each of the heavy chains includes on its surface at least three cysteine residues (third amino acid residues);
(b1-7) PFP-PEG2-azide or PFP-PEG5-azide (AAR linker) was added at 4° C., to generate a linker residue including a BL residue which covalently bonded to the lysine residues (the second amino acid residues) via the BL residue.
L-Histidine stock was added to stop the reaction. Centrifuge and collect supernatant.
Transfer the supernatant to the storge buffer containing L-Histidine by Amicon-15 50 kD. The conjugated anti-GPC3 mAb were reduced with a buffer containing guanidine HC1 and DTT. Centrifuge and collect supernatant. Transfer the supernatant to the storge buffer containing L-Histidine by Amicon-15 50 kD for subsequent LC-MS analysis.

The LC-MS data of bi-functional linker (L) PFP-PEG2-azide- and PFP-PEG5-azide-conjugated anti-GPC3 mAbs were deconvoluted; and the corresponding native and conjugated light chain (LC and LC+1L, LC+2L) and heavy chain (HC) were identified. The peak area of specifically and non-specifically conjugated light chain were divided by the total peak area of light chain to acquire the corresponding ratio, respectively.

Please refer to FIG. 25A and FIG. 25B, which are the Liquid chromatography-mass spectrometry (LC-MS) results of the antibody conjugated with different PFP-based linker (PEG2 group: using PFP-PEG2-N3 as PFP-based linker; PEG5 group: using PFP-PEG5-N3 as PFP-based linker). FIG. 25B indicates that compared with PFP-PEG5-N3, antibody conjugations prepared with PFP-PEG2-N3 as an AAR linker has much more specifically conjugated light chain. This is an unexpected result and reveals that in the case of using PFP-(PEG)n-N3 linker to prepare DAR10 antibody drug conjugation, method of preparing antibody conjugations with PFP-(PEG)n-N3 as an AAR linker leads to higher yield rate (because it leads to more specifically conjugated light chains) if n is a smaller integer (such as an integer from 0 to 2).

### Embodiment 8: Effect of Linkers and Linker-Payloads on the Aggregation During the Preparation of Dual-Payload Antibody Drug Conjugates

The aggregation of antibody-drug conjugates (ADCs) has several significant disadvantages, especially in their safety, efficacy, manufacturability, and regulatory approval. Therefore, the aggregation of antibody-drug conjugates, especially in the dual-payload antibody drug conjugates of the present disclosure, should be prevented.

Size exclusion chromatography HPLC method (SEC-HPLC) was applied for the dual-payload antibody drug conjugate (DAR 10) prepared according to the process described in the embodiments 1-1, 2-1, 3-1, 4-1, 5-1, and 6-1 of the present disclosure and other tested dual-payload antibody drug conjugates (DAR10).

The results revealed that some DAR10 dual-payload antibody drug conjugates (ADCs) exhibited increased aggregation. However, the DAR10 dual-payload antibody drug conjugates of the present disclosure (such as prepared according to the process described in the embodiments 1-1, 2-1, 3-1, 4-1, 5-1, and 6-1, or prepared according to Embodiments 8-1 and 8-2 shown in Table 3) were resistant to protein aggregation. Therefore, the linkers or/and linker-payloads disclosed by the present invention exhibit unexpected results for maintaining the DAR10 antibody drug conjugates in a non-aggregated state.

TABLE 3

| AD2C | | First functional | |
|---|---|---|---|
| embodiments | Antibody | unit | Second functional unit |
| 8-1 | Anti-GPC3 mAb | Formula 125 | Formula 126 |
| 8-2 | Anti-GPC3 mAb | Formula 125 | Formula 127 |

[Formula 125] 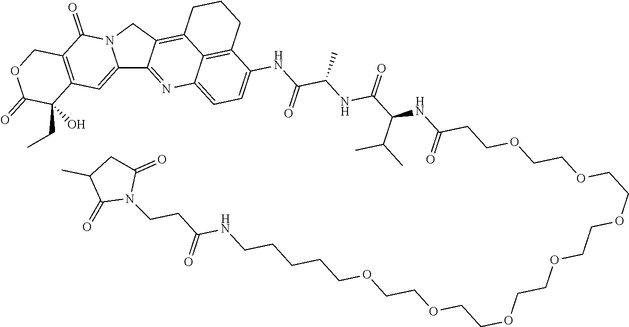 [SMILES: CC[C@@]1(O)C(=O)OCc2c1cc1n(c2=O)Cc2c-1nc1ccc(NC(=O)[C@H](C)NC(=O)[C@@H](NC(=O)CCOCCOCCOCCOCCOCCOCCOCCCCCNC(=O)CCN3C(=O)CC(C)C3=O)C(C)C)c3c1c2CCC3]
[Formula 126] 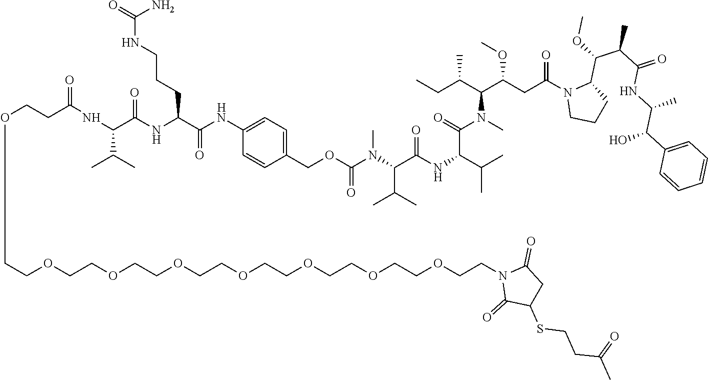 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCOCCOCCOCCOCCOCCOCCOCCN2C(=O)CC(SCCC(C)=O)C2=O)C(C)C)cc1)C(C)C]
[Formula 127]

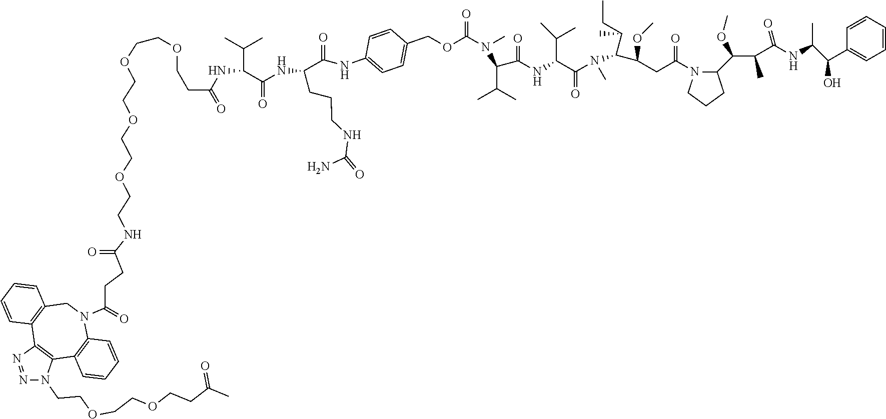 [SMILES: CC[C@H](C)[C@H]([C@H](CC(=O)N1CCCC1[C@@H](OC)[C@H](C)C(=O)N[C@@H](C)[C@H](O)c1ccccc1)OC)N(C)C(=O)[C@H](NC(=O)[C@@H](C(C)C)N(C)C(=O)OCc1ccc(NC(=O)[C@H](CCCNC(N)=O)NC(=O)[C@H](NC(=O)CCOCCOCCOCCOCCNC(=O)CCC(=O)N2Cc3ccccc3-c3nnn(CCOCCOCCC(C)=O)c3-c3ccccc32)C(C)C)cc1)C(C)C]

What is claimed is:

1. A conjugate, comprising:
an antibody or an antigen-binding fragment thereof, comprising a light chain having a first amino acid residue and a second amino acid residue, and a heavy chain having at least one third amino acid residue;
a first functional unit having a formula -L1-D1 conjugated to the antibody or antigen-binding fragment thereof via the first amino acid residue, or via both the first and the at least one third amino acid residues; and
a second functional unit having a formula -L2-D2 conjugated to the antibody or antigen-binding fragment thereof via the second amino acid residue,
wherein
D1 and D2 are each independently a functional moiety;
L1 is a first linker residue connecting D1 to the first amino acid residue, or to both the first and the at least one third amino acid residues;
L2 is a second linker residue connecting D2 to the second amino acid residue;
the first amino acid residue and the second amino acid residue are of different types; and
the first amino acid residue and the at least one third amino acid residue are of a same type.

2. The conjugate according to claim 1, wherein a numerical ratio of the second functional unit to the first functional unit is from 1:1 to 1:8.

3. The conjugate according to claim 1, wherein:
the first amino acid residue and the at least one third amino acid residue are cysteine residues;
the second amino acid residue is a lysine residue or a tyrosine residue;
L1 is covalently conjugated to a sulfur atom of the cysteine residues; and
L2 is covalently conjugated to a nitrogen atom of the lysine residue or a carbon atom of an aromatic ring of the tyrosine residue.

4. The conjugate according to claim 3, wherein the light chain further comprises a fourth amino acid residue, and the conjugate further comprises:
a third functional unit having a formula -L3-D3 conjugated to the antibody or antigen-binding fragment thereof via the fourth amino acid residue,
wherein:
L3 is a third linker residue connecting D3 to the fourth amino acid residue;
D1, D2, and D3 are each independently a functional moiety;
the fourth amino acid residue is of a different type from the first, second, and third amino acid residues; and
the fourth amino acid residue is a tyrosine residue.

5. The conjugate according to claim 1, wherein the functional moiety is selected from the group consisting of an anti-neoplastic agent, an immune-modulating agent, an anti-cancer agent, a chemotherapeutic drug, a DNA-modifying agent, a DNA-damaging agent, a tubulin inhibitor, a proteolysis targeting chimera (PROTAC), a photoimmunotherapy drug, an active agent, a therapeutic moiety, a fluorophore, a nanoparticle, a spin label, a radioactive moiety, a photocaged moiety, an enzyme, and an imaging agent.

6. The conjugate according to claim 1, wherein:
L1 has a structure represented by the formula: -TR-(Y)m1-(PEG)m2-(Z)m3-(AA)m4-(Spacer)m5-; and
L2 has a structure represented by the formula: -(BL)-(PEG)n1-(W)n2-(PEG)n3-(CC1)n4-(CC2)n4-(PEG)n5-(NH—C(═O)CH₂—O—CH₂)n19-(C(═O))n14-(AA)n6-(Spacer)n7-;
wherein:
TR is a residue comprising a sulfhydryl-reactive group conjugated to one of the first and the at least one third amino acid residues;
Y is:
(a) a residue comprising an alkyl group, a cycloalkyl group, a branched alkyl group, a glucuronide, a disulfide, an ester, a hydrazone, a thioether, or any combination thereof,
(b) —(CH₂)m6-(C═O)—; or
(c) —(CH₂)m7-(C═O)—NH—(CH₂)m8-,
wherein m6, m7 and m8 are independently integers ranging from 0 to 10;
PEG represents —CH₂—CH₂—O—;
Z is a residue comprising an alkyl group, a pH-sensitive structure, a hydrazone, a disulfide, a glucuronide, a pyrophosphate, —(C═O)—, or any combination thereof,
AA is an amino acid residue;
Spacer is a self-immolative moiety connecting to D1 or D2, and is selected from the group consisting of a hemiaminal-related molecule, a heterocyclic molecule, a Mannich-type molecule, para-aminobenzyloxycarbonyl (PABC), and any of the aforementioned molecules substituted with a hydrophobicity-masking entity;
BL is a single bond or a linker residue comprising an amino acid reactive moiety conjugated to the second amino acid residue;
W is a linker residue selected from the group consisting of —C(═O)—(CH₂)n8-S-(Succinimid-3-yl-N)—(CH₂)n16-, —C(═O)—(CH₂)n10-, maleimidocaproic acid, an alkyne, a 3-(pyridin-2-yldisulfanyl)propanoate (PDP) residue, an azide, an alkene, an ester, a sulfhydryl group, a hydroxyl group, a thiol, an aldehyde, a ketone, a photoreactive moiety, a glucuronide, a glucurunoside, —S—, and —S—R³—, wherein n8, n10, and n16 are independently integers ranging from 0 to 10, -(Succinimid-3-yl-N)— is represented by the following formula: 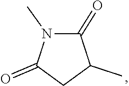
and R³ is selected from the group consisting of C₁₋₆ alkyl, C₁₋₆ alkoxy, halo, halo(C₁₋₆)alkyl, hydroxy, C₁₋₆ alkylthio, C₁₋₆ alkoxycarbonyl, cyano, nitro, amino, mono- or di-C₁₋₆ alkylamino, C₁₋₆ acylamino, C₁₋₆ acyl, carboxy, C₁₋₆ alkoxycarbonyl, C₁₋₆ alkylsulfonyl, C₁₋₆ alkylsulfonamido, and phenyl or substituted phenyl;
CC1 and CC2 are each independently a reaction moiety of a bioorthogonal pair;
m1, m5, n2, n4, n7, n14 and n19 are each independently 0 or 1;
m2, m4, n1, n3, n5 and n6 are independently integers ranging from 0 to 30; and
m3 is an integer from 0 to 10.

7. The conjugate according to claim 6, wherein TR is a maleimide, a maleimide residue, or -(Succinimid-3-yl-N)—.

8. The conjugate according to claim 6, wherein Y is selected from the group consisting of —CH₂CH₂—, —CH₂CH₂CH₂—, —CH₂CH₂CH₂CH₂CH₂—, 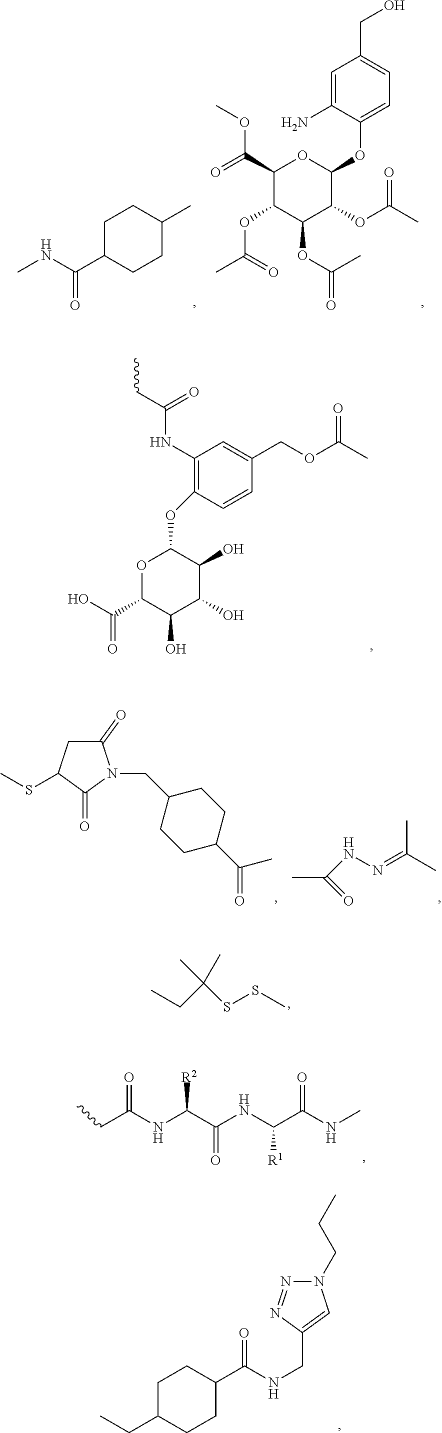
—(CH₂)m6-(C═O)—, and —(CH₂)m7-(C═O)—NH—(CH₂)m8-,
wherein R¹ and R² are each independently selected from the side chains of naturally occurring α-amino acids and non-naturally occurring analogs thereof, including hydrogen, C₁₋₆ straight or branched alkyl, hydroxy-substituted C₁₋₆ alkyl, thio-substituted C₁₋₆ alkyl, carboxy-substituted C₁₋₆ alkyl, amino-substituted C₁₋₆ alkyl, arylmethyl, heteroarylmethyl, and guanidino-, imidazolyl-, or indolyl-containing side chains.

9. The conjugate according to claim 6, wherein Z is selected from the group consisting of —(C═O)—, 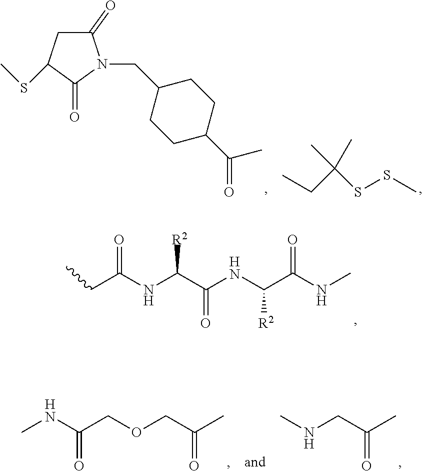
wherein R¹ and R² are each independently selected from the side chains of naturally occurring α-amino acids and non-naturally occurring analogs thereof, including hydrogen, C₁₋₆ straight or branched alkyl, hydroxy-substituted C₁₋₆ alkyl, thio-substituted C₁₋₆ alkyl, carboxy-substituted C₁₋₆ alkyl, amino-substituted C₁₋₆ alkyl, arylmethyl, heteroarylmethyl, and guanidino-, imidazolyl-, or indolyl-containing side chains.

10. The conjugate according to claim 6, wherein Spacer is selected from the group consisting of 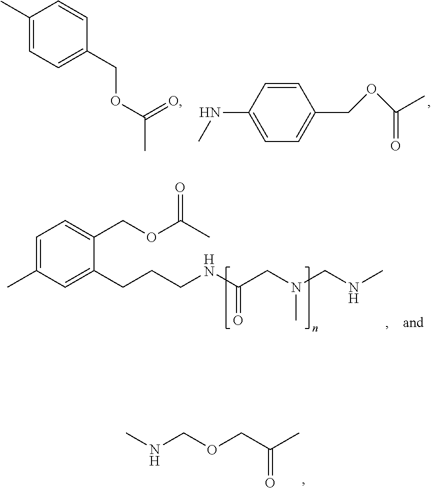
wherein n is greater than or equal to 1.

11. The conjugate according to claim 6, wherein the linker residue is PTDA residue is represented by the following formula: 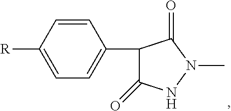
wherein R in the formula of the PTDA residue is —O—(CH₂)n15-, and n15 is an integer from 0 to 30.

12. The conjugate according to claim 6, wherein W is selected from the group consisting of —C(═O)—(CH₂)n8-S-(Succinimid-3-yl-N)—, —C(═O)—(CH₂)n10-, —S—CH₂—CH₂—, —S—CH₂—, —S—, and —S—R³—,
wherein R³ is a residue comprising an alkyl group, a cycloalkyl group, or a branched alkyl group.

13. The conjugate according to claim 6, wherein the bioorthogonal pair is selected from the group consisting of oxime/hydrazone ligation, a Pictet/Spengler ligation, an amino benzamidoxime ligation, a Staudinger ligation, an azide-alkyne cycloaddition reaction, a strain-promoted 1,3-dipolar cycloaddition reaction, a strain-promoted cycloaddition reaction, a tetrazine-based Inverse-electron demand diels-alder reactions with strained alkenes and alkynes, a photo-induced 1,3-dipolar cycloadditions of 2,5-diaryltetrazoles and alkenes, —S—/(Succinimid-3-yl-N)—R⁴, and N₃—R⁵/DBCO-R⁶;
wherein:
DBCO-R⁶ is represented by the following formula: 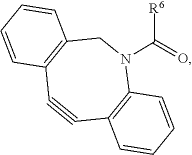
wherein R⁶ is a residue comprising an alkyl group, a polymer of PEG group, a glycuronate group, or —(CH₂)n11-C(═O)—(NH)n12-(CH₂)n13-, and n11, n12 and n13 are independently integers from 0 to 30;
R⁵ is a single bond or a residue comprising a halide phenyl group, an NHS ester, a PTAD group, a —CH₂CO₂—NHS group, an aminoacetic acid-NHS ester group, a succinimidyl carbonate group, a pegylated halide phenyl group, a pegylated NHS ester, a pegylated —CH₂CO2-NHS group, an pegylated aminoacetic acid-NHS ester group, a pegylated succinimidyl carbonate group, a fluorophenyl ester group, a pegylated fluorophenyl ester group, an acid group, a pegylated acid group, a hydrazine group, or a maleimide group; and
(Succinimid-3-yl-N)—R⁴ is represented by one of the following structures:

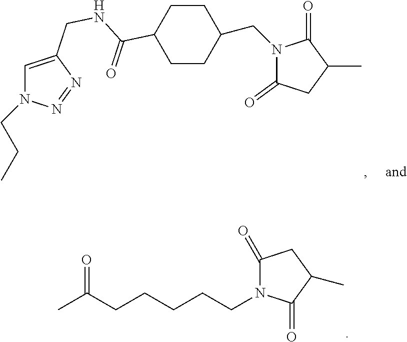

14. The conjugate according to claim 1, wherein the first functional unit is selected from the group consisting of

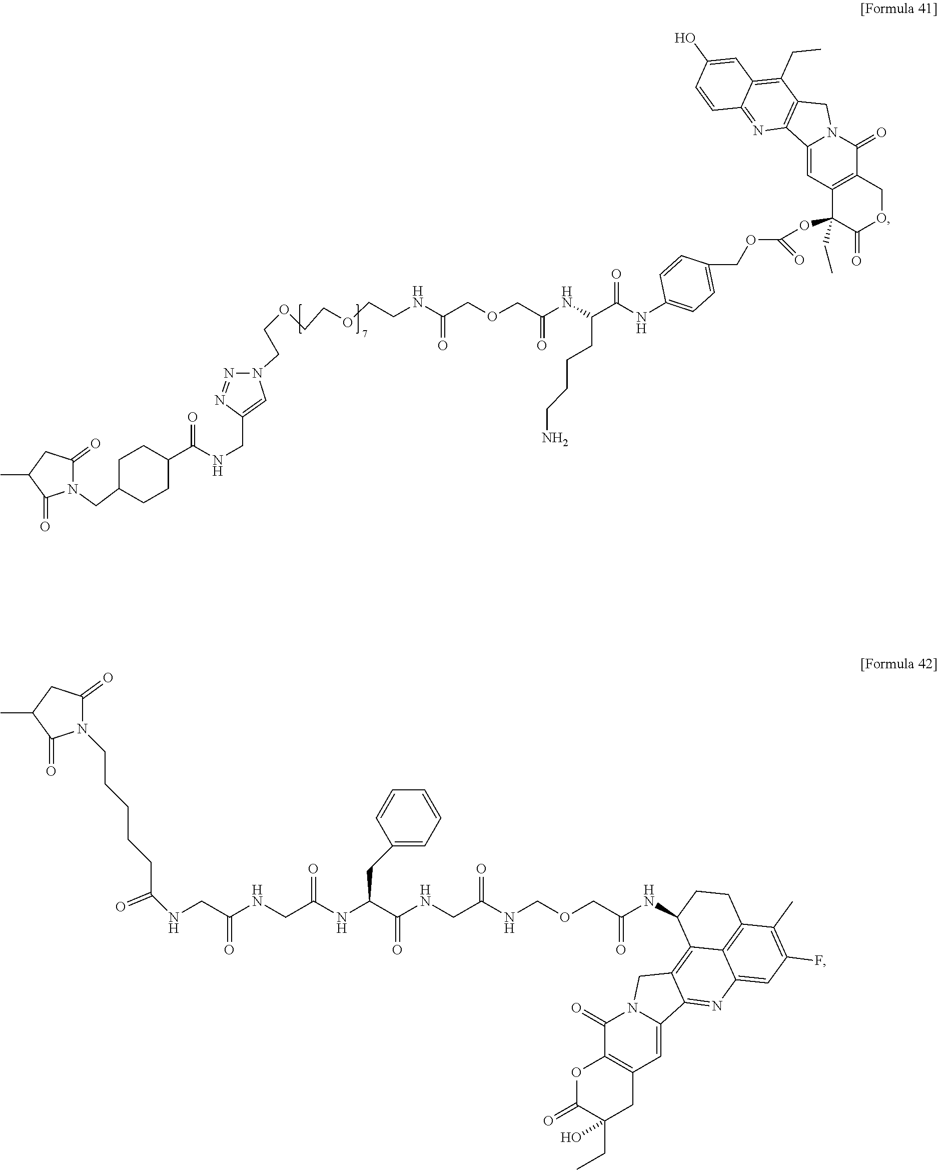

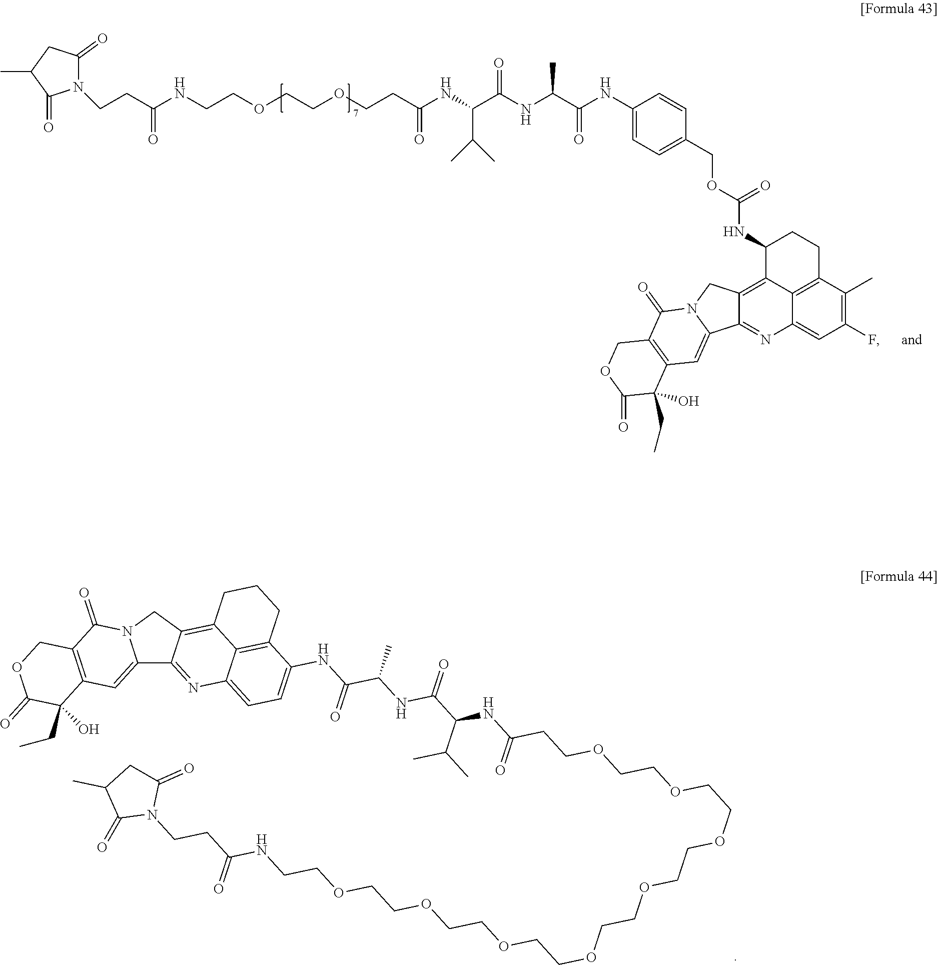

15. The conjugate according to claim 1, wherein the second functional unit is selected from the group consisting of

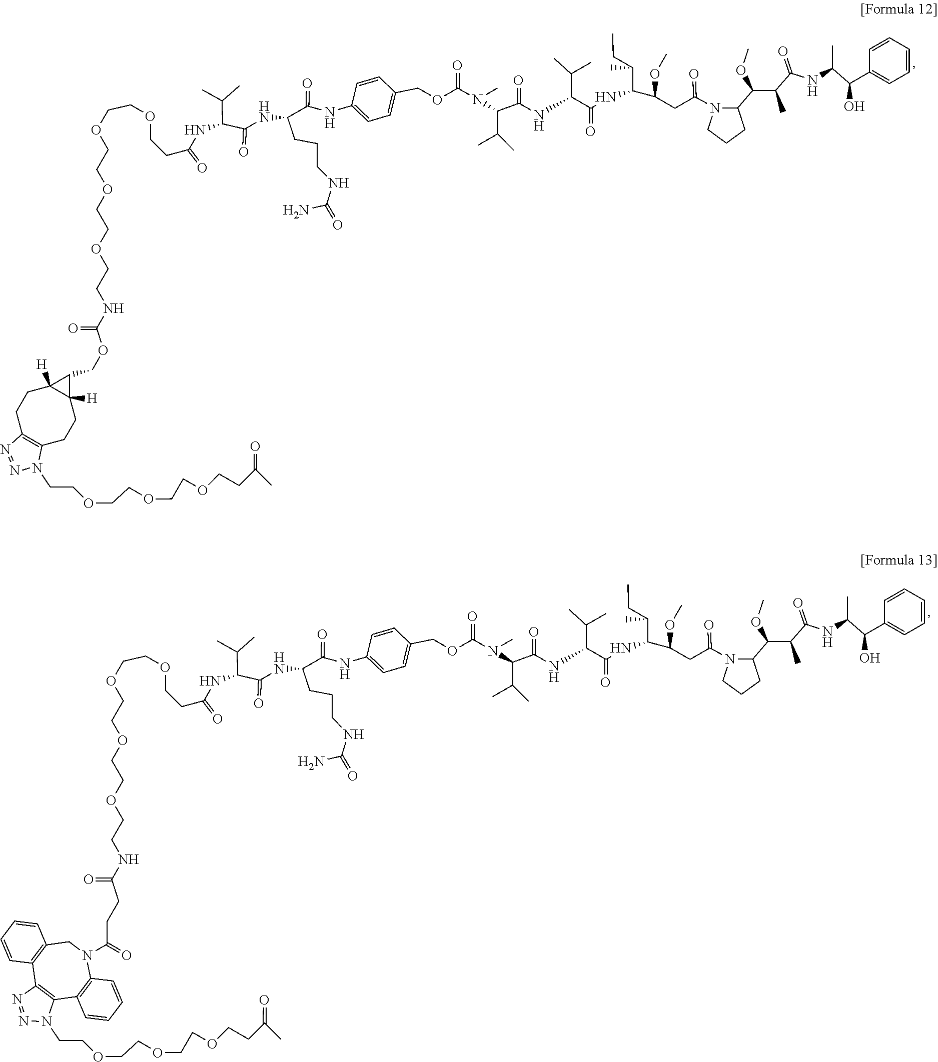

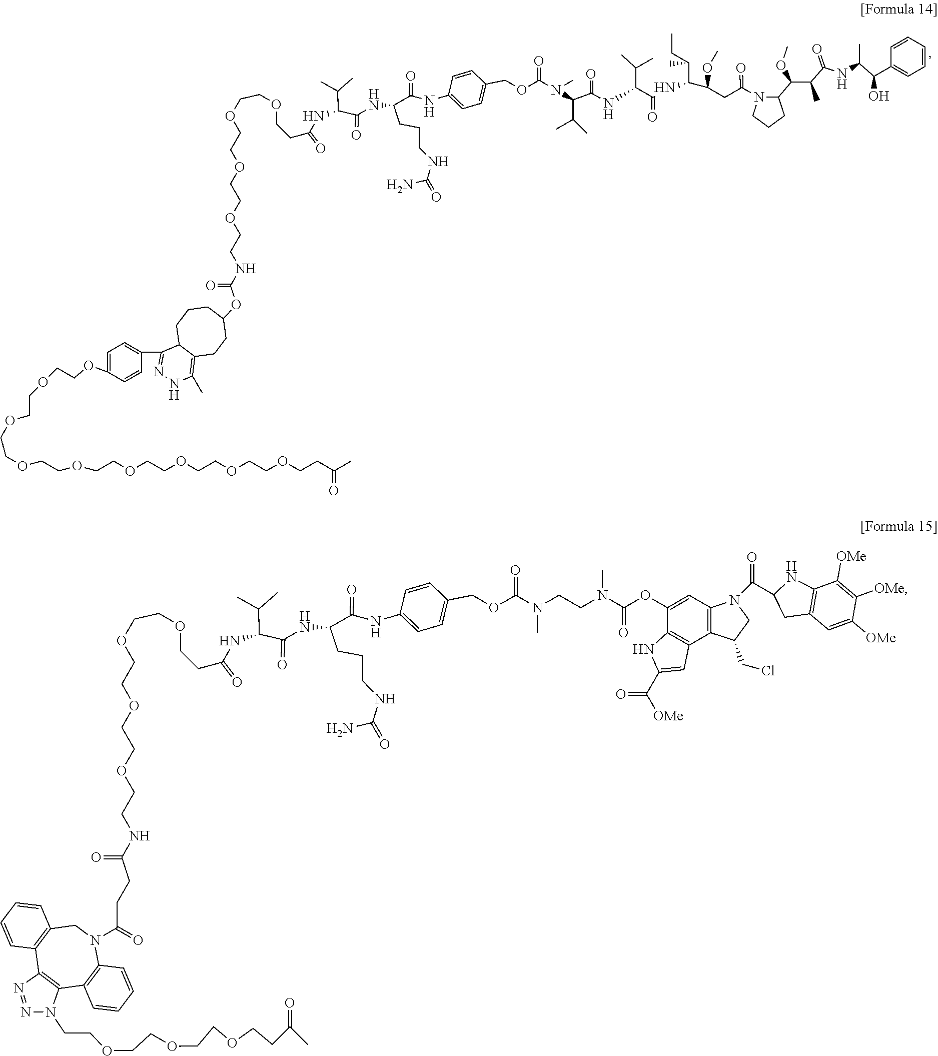

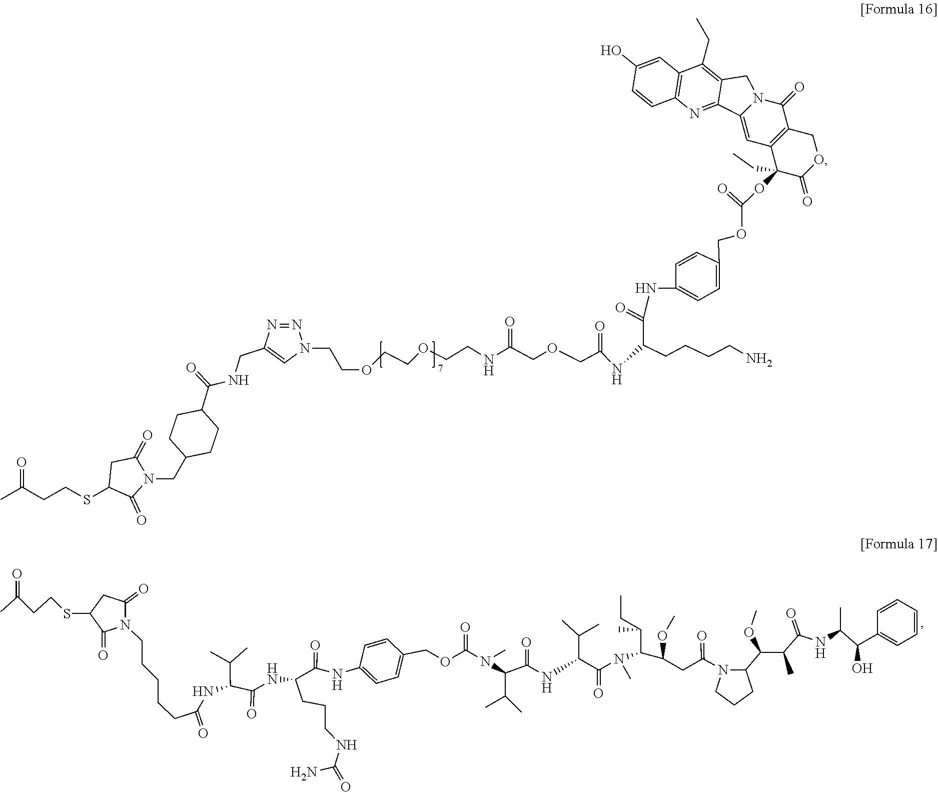

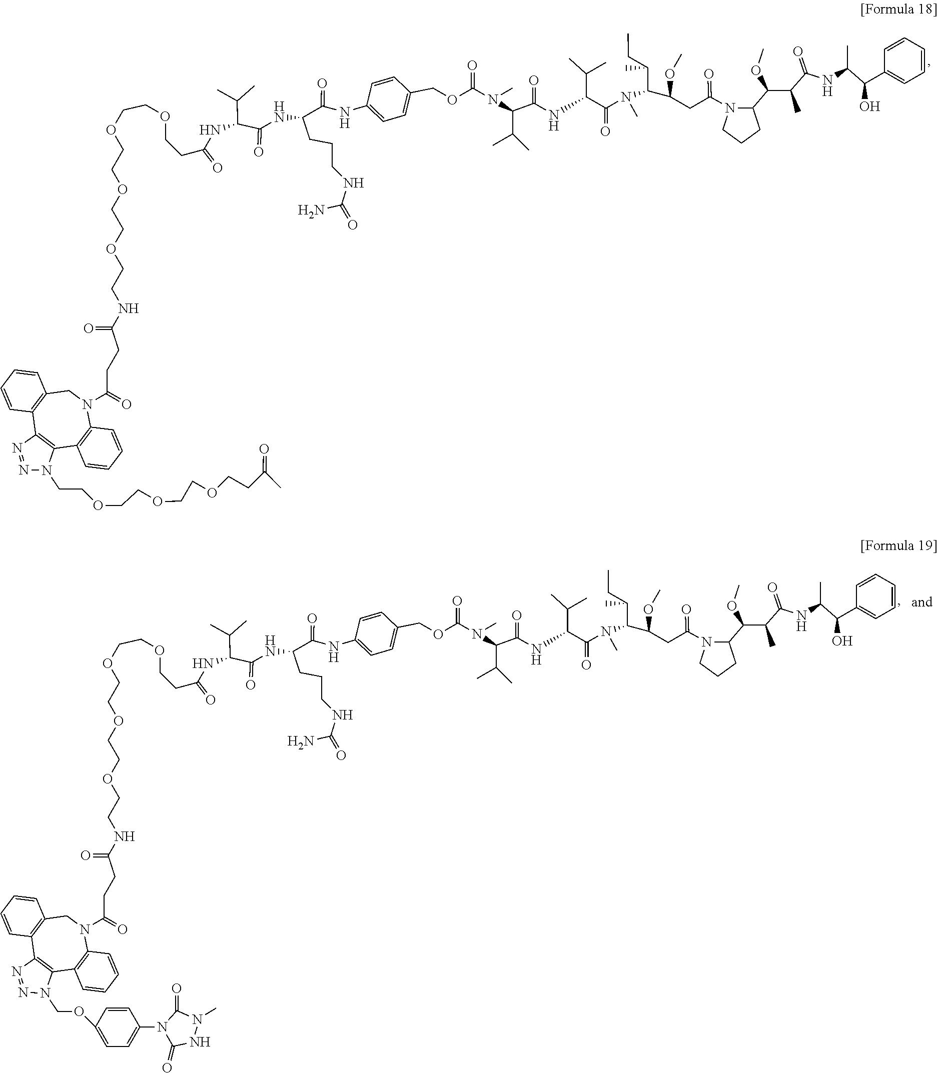

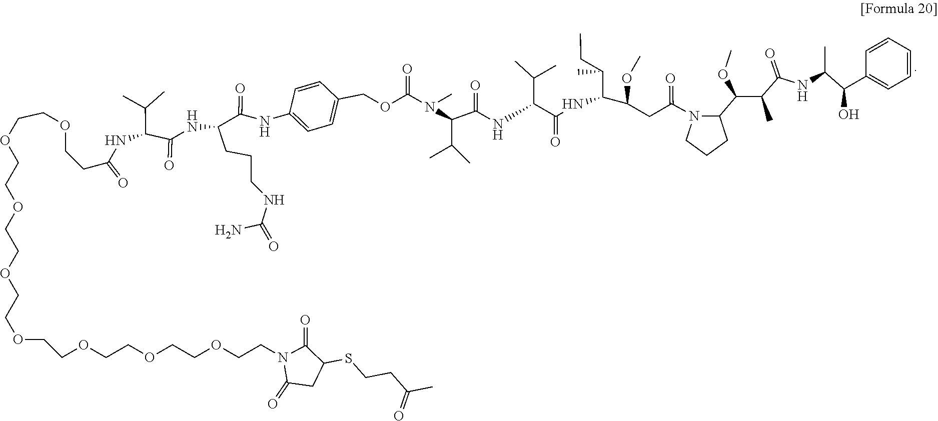 [SMILES: CC[C@H](C)[C@@H](NC(=O)[C@H](NC(=O)[C@@H](C(C)C)N(C)C(=O)OCc1ccc(NC(=O)[C@H](CCCNC(N)=O)NC(=O)[C@H](NC(=O)CCOCCOCCOCCOCCOCCOCCOCCOCCN2C(=O)CC(SCCC(C)=O)C2=O)C(C)C)cc1)C(C)C)[C@H](CC(=O)N1CCCC1[C@@H](OC)[C@H](C)C(=O)N[C@@H](C)[C@H](O)c1ccccc1)OC]

16. The conjugate according to claim 1, wherein the antibody or antigen-binding fragment thereof is selected from the group consisting of an anti-PDL1 antibody, an anti-HER2 antibody, and an anti-GPC3 antibody, and the light chain is a kappa light chain.

17. The conjugate according to claim 16, wherein the second functional unit is conjugated to the kappa light chain at a residue selected from the group consisting of Lys188, Lys190, and a combination thereof.

18. The conjugate according to claim 1, wherein a molecular weight of each of the first functional unit and the second functional unit is 0.5 to 20 kDa.

19. A pharmaceutical composition comprising the conjugate according to claim 1 or a salt thereof, and a pharmaceutically acceptable carrier.

20. A method of treating a disease or disorder in a subject in need thereof, comprising administering to the subject an effective amount of the conjugate according to claim 1,
wherein the disease or the disorder comprises a tumor, a cancer, an autoimmune disease, a neuronal disease, a hematopoietic cell-related disease, a metabolic syndrome, a pathogenic disease, a viral infectious disease, a fungal infectious disease, a protozoan infectious disease, a bacterial infectious disease, or any combination thereof.

21. A method of manufacturing a conjugate, comprising:
providing an antibody or antigen-binding fragment thereof, wherein the antibody or antigen-binding fragment thereof comprises a light chain having a first amino acid residue and a second amino acid residue, and a heavy chain having at least one third amino acid residue; and the first amino acid residue and the second amino acid residue are of different types, and the first amino acid residue and the at least one third amino acid residue are of a same type;
conjugating a first functional unit to the first amino acid residue and the at least one third amino acid residue, wherein the first functional unit comprises a sulfhydryl-reactive group and has a formula -L1-D1, by the steps of:
(a1) reducing the antibody or antigen-binding fragment thereof to expose free thiol groups on the first amino acid residue and the at least one third amino acid residue; and
(a2) reacting the first functional unit with the free thiol groups exposed in step (a1) to form a covalent bond between each free thiol group and the sulfhydryl-reactive group of the first functional unit; and
conjugating a second functional unit having a formula -L2-D2 to the second amino acid residue by the steps of:
(b1) reacting an AAR linker comprising an AAR group with the antibody or antigen-binding fragment thereof to produce an intermediate conjugate comprising a linker residue covalently bonded to the second amino acid residue, wherein the intermediate conjugate does not comprise the AAR group; and
(b2) contacting a molecule comprising a functional moiety with the intermediate conjugate of the step (b1) to form a covalent linkage between the molecule and the linker residue of the intermediate conjugate via the reaction of reaction moieties of a bioorthogonal pair, thereby producing the second functional unit represented by the formula -L2-D2,
wherein,
L1 is a first linker residue, L2 is a second linker residue, and D1 and D2 are each independently a functional moiety.

22. The method of claim 21, wherein:
the AAR linker comprises a structure of formula: (AAR)-(PEG)n1-(W)n2-(PEG)n3-(CC1)n4;
L1 has a structure of formula: TR-(Y)m1-(PEG)m2-(Z)m3-(AA)m4-(Spacer)m5-;
L2 has a structure of formula: (BL)-(PEG)n1-(W)n2-(PEG)n3-(CC1)n4-(CC2)n4-(PEG)n5-(NH—C(═O)CH₂—O—CH₂)n19-(C(═O))n14-(AA)n6-(Spacer)n7-;
wherein:
TR is a residue comprising a sulfhydryl-reactive group configured to conjugate to the first amino acid residue and the at least one third amino acid residue;
Y is:
a residue comprising an alkyl group, a cycloalkyl group, a branched alkyl group, a glucuronide, a disulfide, an ester, a hydrazone, a thioether, or any combination thereof,
—(CH₂)m6-(C═O)—; or
—(CH₂)m7-(C═O)—NH—(CH₂)m8-,
wherein each of m6, m7 and m8 is independently an integer ranging from 0 to 10;
PEG represents —CH₂—CH₂—O—;
Z is a residue comprising an alkyl group, a pH-sensitive structure, a hydrazone, a disulfide, a glucuronide, a pyrophosphate, —(C═O)—, or any combination thereof,
AA is an amino acid residue;
AAR represents the AAR group selected from the group consisting of: 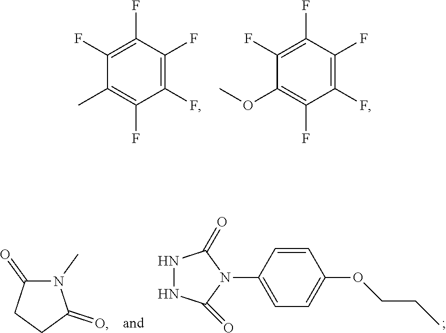
W is a functional linker residue selected from the group consisting of —C(═O)—(CH₂)n8-S-(Succinimid-3-yl-N)n16-, —C(═O)—(CH₂)n10-, maleimidocaproic acid, an alkyne, a 3-(pyridin-2-yldisulfanyl) propanoate (PDP) residue, an azide, an alkene, an ester, a sulfhydryl group, a hydroxyl group, a thiol, an aldehyde, a ketone, a photoreactive moieties, a glucuronide, a glucurunoside, —S—, or —S—R³—, wherein n8 and n10 are independently integers ranging from 0 to 10, -(Succinimid-3-yl-N)— is represented by the following formula: 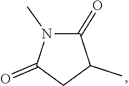
and R³ is selected from the group consisting of C₁₋₆ alkyl, C₁₋₆ alkoxy, halo, halo(C₁₋₆)alkyl, hydroxy, C₁₋₆ alkylthio, C₁₋₆ alkoxycarbonyl, cyano, nitro, amino, mono- or di-C₁₋₆ alkylamino, C₁₋₆ acylamino, C₁₋₆ acyl, carboxy, C₁₋₆ alkoxycarbonyl, C₁₋₆ alkylsulfonyl, C₁₋₆ alkylsulfonamido, and phenyl or substituted phenyl;
CC1 and CC2 are each independently a reaction moiety of a bioorthogonal pair;
BL is a single bond or a linker residue comprising an amino acid reactive moiety configured to conjugate the second amino acid residue;
Spacer is a self-immolative moiety connecting to D1 or D2, selected from the group consisting of a hemiaminal-related molecule, a heterocyclic molecule, a Mannich-type molecule, para-aminobenzyloxycarbonyl (PABC), and any of the aforementioned molecules substituted with a hydrophobicity-masking entity;
m1, m5, n2, n4, n7, n14 and n19 are each independently 0 or 1;
m2, m4, n1, n3, n5 and n6 are independently integers ranging from 0 to 30; and
m3 is an integer from 0 to 10.

23. The method of claim 22, wherein the AAR linker is a PFP-based linker comprising a disulfide bond or an azide functional group.

24. The method of claim 23, wherein the PFP-based linker is selected from the group consisting of a propargyl-SS-PFP ester, a 2,3,4,5,6-pentafluorophenyl 4-(pyridin-2-yldisulfanyl)butanoate (PDB-PFP), a 2,3,4,5,6-pentafluorophenyl 3-(pyridin-2-yldisulfanyl)propanoate (PDP-PFP), a perfluorophenyl 3-(pyridin-2-yldisulfanyl)propanoate, perfluorophenyl 4-methyl-4-(pyridine-2-yldisulfanyl)pentanoate, and a perfluorophenyl 4-(pyridin-2-yldisulfanyl)butanoate.

25. The method of claim 22, wherein the structure in the AAR linker is selected from the group consisting of:

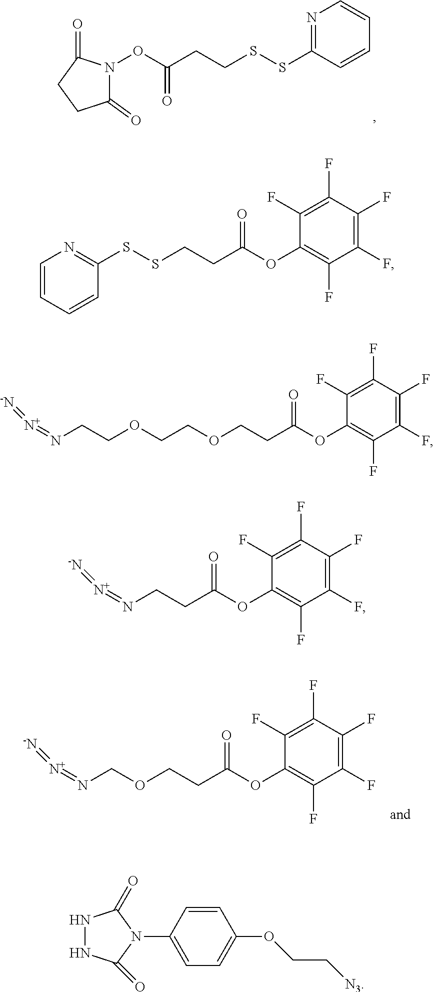

26. The method of claim 22, wherein CC1 is selected from the group consisting of

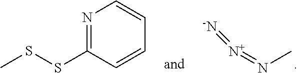

27. The method according to claim 21, wherein D1 and D2 are independently selected from the group consisting of an anti-neoplastic agent, an immune-modulating agent, an anti-cancer agent, a chemotherapeutic drug, a DNA-modifying agent, a DNA-damaging agent, a tubulin inhibitor, a proteolysis targeting chimera (PROTAC), a photoimmunotherapy drug, an active agent, a therapeutic moiety, a fluorophore, a nanoparticle, a spin label, a radioactive moiety, a photocaged moiety, an enzyme, and an imaging agent.

28. The method according to claim 21, wherein:
the first amino acid residue and the at least one third amino acid residue are a cysteine residue;
the second amino acid residue is a lysine residue or a tyrosine residue,
L1 is covalently linked to a sulfur atom in the cysteine residue; and
L2 is covalently linked to a nitrogen atom in the lysine residue or a carbon atom in an aromatic ring of the tyrosine residue.

29. The method according to claim 21, further comprising reducing the intermediate conjugate produced in step (b1) to expose a free thiol group thereon prior to step (b2);
wherein the covalent linkage formed in step (b2) is between said free thiol group of the intermediate conjugate and a sulfhydryl-reactive group of the molecule comprising the functional moiety.

30. The method according to claim 21, wherein the covalent linkage formed in step (b2) wherein the reaction moiety of the bioorthogonal pair on the intermediate conjugate is one of an azide group and an alkyne group, and the reaction moiety of the bioorthogonal pair on the molecule is the other of an azide group and an alkyne group.
